(19) United States
(12) Patent Application Publication
(10) Pub. No.: US 2021/0047689 A1
(43) Pub. Date: Feb. 18, 2021

(54) PRECISION MEDICINE FOR PAIN: DIAGNOSTIC BIOMARKERS, PHARMACOGENOMICS, AND REPURPOSED DRUGS

(71) Applicants: Indiana University Research and Technology Corporation, Indianapolis, IN (US); The United States Government as Represented by the Department of Veterans Affairs, Washington, DC (US)

(72) Inventor: Alexander Bogdan Niculescu, Indianapolis, IN (US)

(73) Assignees: Indiana University Research and Technology Corporation, Indianapolis, IN (US); The United States Government as Represented by the Department of Veterans Affairs, Washington, DC (US)

(21) Appl. No.: 16/963,479

(22) PCT Filed: Mar. 14, 2019
(86) PCT No.: PCT/US19/22305

§ 371 Date: Jul. 20, 2020

Related U.S. Application Data
(60) Provisional application No. 62/642,789, filed on Mar. 14, 2018.

(51) Int. Cl. C12Q 1/6883; G01N 33/68; G16B 25/10; G16B 40/00

(52) U.S. Cl. CPC C12Q 1/6883; G01N 33/6893; G16B 25/10; G01N 2800/52; C12Q 2600/106; C12Q 2600/158; G01N 2800/2842; G16B 40/00

(57) ABSTRACT

Disclosed are methods for treating pain and tracking response to treatment. Also disclosed are methods for determining pain, including predicting future medical care facility visits for pain.

### CROSS-REFERENCE TO RELATED APPLICATION

This application claims priority to U.S. Provisional Application Ser. No. 62/642,789, filed Mar. 14, 2018, which is hereby incorporated by reference in its entirety.

### STATEMENT OF GOVERNMENT SUPPORT

This invention was made with government support under OD007363 awarded by the National Institutes of Health and CX000139 merit award by the Veterans Administration. The government has certain rights in the invention.

### BACKGROUND OF THE DISCLOSURE

The present disclosure relates generally to methods for objectively determining and predicting pain. More particularly, the present disclosure relates to methods for tracking pain intensity, predicting levels of pain and predicting future medical facility visits for pain. Also disclosed are drugs and natural compounds identified as candidates for treating pain using biomarker gene expression signatures.

Pain is a subjective sensation that reflects bodily damage and the possibility of future harm. Pain treatment is a multi-billion dollar market in the United States. The United States is, however, experiencing an opioid abuse epidemic.

Mental states can affect the perception of pain, and in turn, can be affected by pain. Psychiatric patients may have an increased perception of pain, as well as increased physical health reasons for pain due to their often adverse life trajectory.

Currently, there are no objective tests for determining pain, so clinicians must rely on self-reporting by patients. An objective test for pain can facilitate proper diagnosis and treatment, enabling more confident treatment for those needing treatment for pain, and avoid over-prescribing of potentially addictive medications to those not in need. Blood biomarkers for pain can serve as companion diagnostics for clinical trials for the development of new pain medications and repurposing existing drugs for use as pain treatments. Accordingly, there exists a need for objective measures for determining pain, which can guide appropriate treatment.

### SUMMARY OF THE DISCLOSURE

The present disclosure relates generally to methods for determining and predicting pain. More particularly, the present disclosure relates to methods for objectively determining pain intensity, predicting future emergency department (ED) visits for pain. Also disclosed are methods for identifying drug and natural compounds as candidates for treating pain using biomarker gene expression signatures.

In one aspect, the present disclosure is directed to a method for determining pain intensity in a subject in need thereof. The method comprises: obtaining an expression level of a blood biomarker in a sample obtained from the subject; obtaining a reference expression level of a blood biomarker; and identifying a difference between the expression level of the blood biomarker in a sample obtained from the subject and the reference expression level of a blood biomarker, wherein the difference in the expression level of the blood biomarker in the sample obtained from the subject and the reference expression level of the blood biomarker determines pain intensity. In one embodiment, the blood biomarker is a panel of blood biomarkers. The reference level can be an average or reference range in the population (a “cross-sectional” approach), or it can be the level of a sample obtained previously in the subject when the subject was not in need of treating pain (a “longitudinal” approach).

In another aspect, the present disclosure is directed to a method for identifying a blood biomarker for pain, the method comprising: obtaining a first biological sample from a subject and administering a first pain intensity test to the subject; obtaining a second biological sample from the subject and administering a second pain intensity test to the subject; identifying a first cohort of subjects by identifying subjects having a change from low pain intensity to high pain intensity as determined by a difference between the first pain intensity test and the second pain intensity test; identifying candidate biomarkers in the first cohort by identifying biomarkers having a change in expression between the first biological sample and the second biological sample.

In one aspect, the present disclosure is directed to a method for predicting future emergency department (ED) visits for pain. The method comprises: obtaining an expression level of a blood biomarker or panel of blood biomarkers in a sample obtained from the subject; obtaining a reference expression level of the blood biomarker or panel of blood biomarkers; identifying a difference in the expression level of the blood biomarkers in the sample and the reference expression level of the blood biomarkers; wherein the difference in the expression level of the blood biomarkers in the sample obtained from the subject and the reference expression level of the blood biomarkers determines the likelihood of future ED visits for pain. In one embodiment, the blood biomarker is a panel of blood biomarkers. The reference expression level can be that as described herein.

In another aspect, the present disclosure is directed to a method for mitigating pain in a subject in need thereof. The method comprises: obtaining an expression level of a blood biomarker in a sample obtained from the subject; obtaining a reference expression level of the blood biomarker; identifying a difference in the expression level of the blood biomarker in the sample and the reference expression level of the blood biomarker; and administering a treatment, wherein the treatment reduces the difference between the expression level of the blood biomarker in the sample and the reference expression level of the blood biomarker to mitigate pain in the subject. In one embodiment, the blood biomarker is a panel of blood biomarkers. The reference expression level can be that as described herein.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A-1G depict Steps 1-3: Discovery, Prioritization and Validation. FIG. 1A depicts Cohorts used in study, depicting flow of discovery, prioritization, and validation of biomarkers from each step. FIG. 1B depicts Discovery cohort longitudinal within-participant analysis. Phchp### is study ID for each participant. V# denotes visit number. FIG. 1C depicts Discovery of possible subtypes of Pain based on High Pain visits in the discovery cohort. Participants were clustered using measures of mood and anxiety (Simplified Affective State Scale (SASS)), as well as psychosis (PANNS Positive) FIG. 1D depicts Differential gene expression in the Discovery cohort-number of genes identified with differential expression (DE) and absent-present (AP) methods with an internal score of 1 and above. Red/Underlined-increased in expression in High Pain, blue/Bold-decreased in expression in High Pain. At the discovery step probesets are identified based on their score for tracking pain with a maximum of internal points of 6 (33% (2pt), 50% (4pt) and 80% (6pt)). FIG. 1E depicts prioritization with CFG for prior evidence of involvement in pain. In the prioritization step probesets are converted to their associated genes using Affymetrix annotation and GeneCards. Genes are prioritized and scored using CFG for pain evidence with a maximum of 12 external points. Genes scoring at least 6 points out of a maximum possible of 18 total internal and external scores points are carried to the validation step. FIG. 1F depicts Validation in an independent cohort of psychiatric patients with co-morbid pain disorders and severe subjective and functional pain ratings. In the validation step biomarkers are assessed for stepwise change from the discovery groups of participants with Low Pain, to High Pain, to Clinically Severe Pain disorder, using ANOVA. N=number of testing visits. 5 biomarkers were nominally significant, MFAP3 and PIK3CD were the most significant, and 68 biomarkers were stepwise changed.

FIGS. 2A-2C depict Best Single Biomarkers Predictors for State Predictions (FIG. 2A), Trait Predictions First Year (FIG. 2B), and Trait Predictions All Future Years (FIG. 2C). From the long list (n=65). Those on short list (n=5) are bolded. Bar graph shows best predictive biomarkers in each group. * Nominally significant p<0.05. ** Bonferroni significant for the 65 biomarkers tested. Table underneath the figures displays the actual number of biomarkers for each group whose ROC AUC p-values were at least nominally significant. Some female diagnostic groups were omitted from the graph as they did not have any significant biomarkers. Cross-sectional was based on levels at one visit. Longitudinal was based on levels at multiple visits (integrates levels at most recent visit, maximum levels, slope into most recent visit, and maximum slope). Dividing lines represent the cutoffs for a test performing at chance levels (white), and at the same level as the best biomarkers for all subjects in cross-sectional (gray) and longitudinal (black) based predictions. All biomarkers performed better than chance. Biomarkers also performed better when personalized by gender and diagnosis.

FIG. 3 depicts the pain scale of male and female psychiatric participants.

FIG. 4 depicts the STRING interaction network for 60 top biomarkers for pain.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

In accordance with the present disclosure, methods have been developed to objectively determine pain intensity and predict future emergency department (ED) visits for pain.

In some embodiments, the methods of the present disclosure as described herein are intended to include the use of such methods in “at risk” subjects, including subjects unaffected by or not otherwise afflicted with pain as described herein, for the purpose of diagnosing, prognosing and identifying subjects such that treatment, treatment planning, and treatment options for pain can be made. As used herein, a subject “at risk for pain” refers to individuals who may develop pain. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified subjects (that is, the subset or subclass of subjects “at risk for” the specific conditions noted herein), not all subjects will fall within the subset or subclass of subjects as described herein.

Particularly suitable subjects are humans Suitable subjects can also be experimental animals such as, for example, monkeys and rodents, that display a behavioral phenotype associated with pain. In one particular aspect, the subject is a female human. In another particular aspect, the subject is a male human.

Suitable samples can be, for example, saliva, blood, plasma, serum and a cheek swab. The samples can be further processed using methods known to those skilled in the art to isolate molecules contained in the sample such as, for example, cells, proteins and nucleic acids (e.g., DNA and RNA).

The isolated molecules can also be further processed. For example, cells can be lysed and subjected to methods for isolating proteins and/or nucleic acids contained within the cells. Proteins and nucleic acids contained in the sample and/or in isolated cells can be processed. For example, proteins can be processed for electrophoresis, Western blot analysis, immunoprecipitation and combinations thereof. Nucleic acids can be processed, for example, for polymerase chain reaction, electrophoresis, Northern blot analysis, Southern blot analysis, RNase protection assays, microarrays, serial analysis of gene expression (SAGE) and combinations thereof.

Suitable probes are described herein and can include, for example, nucleic acid probes, antibody probes, and chemical probes.

In some embodiments, the probe can be a labeled probe. Suitable labels can be, for example, a fluorescent label, an enzyme label, a radioactive label, a chemical label, and combinations thereof. Suitable radioactive labels are known to those skilled in the art and can be a radioisotope such as, for example, ³²P, ³³P, ³⁵S, ³H and ¹²⁵I. Suitable enzyme labels can be, for example, colorimetric labels and chemiluminescence labels. Suitable colorimetric (chromogenic) labels can be, for example, alkaline phosphatase, horse radish peroxidase, biotin and digoxigenin. Biotin can be detected using, for example, an anti-biotin antibody, or by streptavidin or avidin or a derivative thereof which retains biotin binding activity conjugated to a chromogenic enzyme such as, for example, alkaline phosphatase and horse radish peroxidase. Digoxigenin can be detected using, for example, an anti-digoxigenin antibody conjugated to a chromogenic enzyme such as, for example, alkaline phosphatase and horse radish peroxidase. Chemiluminescence labels can be, for example, alkaline phosphatase, glucose-6-phosphate dehydrogenase, horseradish peroxidase, Renilla luciferase, and xanthine oxidase. A particularly suitable label can be, for example, SYBR® Green (commercially available from Life Technologies). A particularly suitable probe can be, for example, an oligonucleotide labelled with SYBR® Green. Suitable chemical labels can be, for example, periodate and 1-Ethyl-3-[3-dimethylaminopropyl]carbodiimide hydrochloride (EDC).

As used herein, “diagnosing” and “diagnosis” are used according to their ordinary meaning as understood by those skilled in the art to refer to determining objectively that a subject has increased pain intensity.

As used herein, “predicting pain in a subject in need thereof” refers to indicating in advance that a subject is likely to develop or is at risk for developing pain and/or identifying that a subject with pain wherein the pain is likely to increase and/or identifying a subject that will visit a hospital or other medical facility because of pain and/or because of increasing pain.

As used herein, the term “biomarker” refers to a molecule to be used for analyzing a subject's test sample. Examples of such biomarkers can be nucleic acids (such as, for example, a gene, DNA and RNA), proteins and polypeptides. In particularly preferred embodiments, the biomarker can be the levels of expression of a biomarker gene. Particularly suitable biomarker genes can be, for example, those listed in Tables 1, 4, 5, 7 and combinations thereof.

As used herein, “a reference expression level of a biomarker” refers to the expression level of a biomarker established for a subject with no pain, expression level of a biomarker in a normal/healthy subject with no pain as determined by one skilled in the art using established methods as described herein, and/or a known expression level of a biomarker obtained from literature. In one suitable embodiment, the reference level can be an average or reference range in the population (a “cross-sectional” approach). In another embodiment, the reference expression level can be the level of a sample obtained previously in the subject when the subject was not in need of treating pain (a “longitudinal” approach). The reference expression level of the biomarker can further refer to the expression level of the biomarker established for a High Pain subject, including a population of High Pain subjects. The reference expression level of the biomarker can also refer to the expression level of the biomarker established for a Low Pain subject, including a population of Low Pain subjects. The reference expression level of the biomarker can also refer to the expression level of the biomarker established for any combination of subjects such as a subject with no pain, expression level of the biomarker in a normal/healthy subject with no pain, expression level of the biomarker for a subject who has pain at the time the sample is obtained from the subject, but who later exhibits increase in pain, expression level of the biomarker as established for a High Pain subject, including a population of High Pain subjects, and expression level of the biomarker can also refer to the expression level of the biomarker established for a Low Pain subject, including a population of Low Pain subjects. The reference expression level of the biomarker can also refer to the expression level of the biomarker obtained from the subject to which the method is applied. As such, the change within a subject from visit to visit can indicate increased or decreased pain. For example, a plurality of expression levels of a biomarker can be obtained from a plurality of samples obtained from the same subject and used to identify differences between the plurality of expression levels in each sample. Thus, in some embodiments, two or more samples obtained from the same subject can provide an expression level(s) of a blood biomarker and a reference expression level(s) of the blood biomarker.

As used herein, “expression level of a biomarker” refers to the process by which a gene product is synthesized from a gene encoding the biomarker as known by those skilled in the art. The gene product can be, for example, RNA (ribonucleic acid) and protein. Expression level can be quantitatively measured by methods known by those skilled in the art such as, for example, northern blotting, amplification, polymerase chain reaction, microarray analysis, tag-based technologies (e.g., serial analysis of gene expression and next generation sequencing such as whole transcriptome shotgun sequencing or RNA-Seq), Western blotting, enzyme linked immunosorbent assay (ELISA), and combinations thereof.

As used herein, a “difference” and/or “change” in the expression level of the biomarker refers to an increase or a decrease in the measured expression level of a blood biomarker when analyzed against a reference expression level of the biomarker. In some embodiments, the “difference” and/or “change” refers to an increase or a decrease by about 1.2-fold or greater in the expression level of the biomarker as identified between a sample obtained from the subject and the reference expression level of the biomarker. In one embodiment, the difference and/or change in expression level is an increase or decrease by about 1.2 fold. As used herein “a risk for pain” can refer to an increased (greater) risk that a subject will experience (or develop) pain. For example, depending on the biomarker(s) selected, the difference and/or change in the expression level of the biomarker(s) can indicate an increased (greater) risk that a subject will experience (or develop) pain. Conversely, depending on the biomarker(s) selected, the difference and/or change in the expression level of the biomarker(s) can indicate a decreased (lower) risk that a subject will experience (or develop) pain.

Methods for Treating Pain

In one aspect, the present disclosure is directed to a method for treating pain in a subject in need thereof. The method includes: obtaining an expression level of a blood biomarker in a sample obtained from the subject; obtaining a reference expression level of the blood biomarker; identifying a difference in the expression level of the blood biomarker in the sample and the reference expression level of the blood biomarker; and administering a treatment, wherein the treatment reduces the difference between the expression level of the blood biomarker in the sample and the reference expression level of the blood biomarker to mitigate pain in the subject.

The biomarkers are selected from the group listed in Tables 1, 4, 5, 7, and combinations thereof. In some embodiments, a panel of blood biomarkers is used. Biomarkers can be selected with different weighting coefficients possible.

Suitable treatments include those listed in Tables 1, 2, 7, and combinations thereof. Suitable treatments further include pain treatments known to those skilled in the art. Particularly suitable treatments include SC-560, pyridoxine, methylergometrine, LY-294002, haloperidol, cytisine, cyanocobalamin, apigenin, betaescin, amoxapine, and combinations thereof.

In some embodiments, the expression level of the blood biomarker in the sample obtained from the subject is decreased as compared to the reference expression level of the biomarker.

In some embodiments, the expression level of the blood biomarker in the sample obtained from the subject is increased as compared to the reference expression level of the biomarker.

In some embodiments, the method further includes performing a neuropsychological test on the subject. Generally, neuropsychological testing includes a comprehensive assessment of cognitive and personality functioning. More particularly, exemplary neuropsychological tests include: for intelligence (e.g., WAIS, WISC, SB, TONI); for achievement (e.g., WJ-III, WIAT, WRAT); for attention (e.g., CCPT, WCST, Vanderbilt, NEPSY); for language (e.g., GORT, Boston Naming, HRB-Aphasia for memory and learning (e.g., WMS, WRAML, CVLT, RAVLT, ROCF, NEPSY); for motor control (e.g., Grooved Pegoard, Finger Tapping, Grip Strength, Lateral Dominance); for visual (e.g., Spatial-ROCFT, Bender-Gestalt, HVOT); for autism (e.g., ADOS, ASDS, ADI, GARS); for executive functioning (e.g., WCST, BRIEF, EFSD, D-KEFS, HRB); and for behavioral (e.g., BASC, Achenbach, Vanderbilt).

### Methods for Determining Pain

In one aspect, the present disclosure is directed to a method for determining High Pain intensity in a subject in need thereof. The method includes: obtaining an expression level of a blood biomarker in a sample obtained from the subject; obtaining a reference expression level of the blood biomarker; and identifying a difference in the expression level of the blood biomarker in the sample and the reference expression level of the blood biomarker.

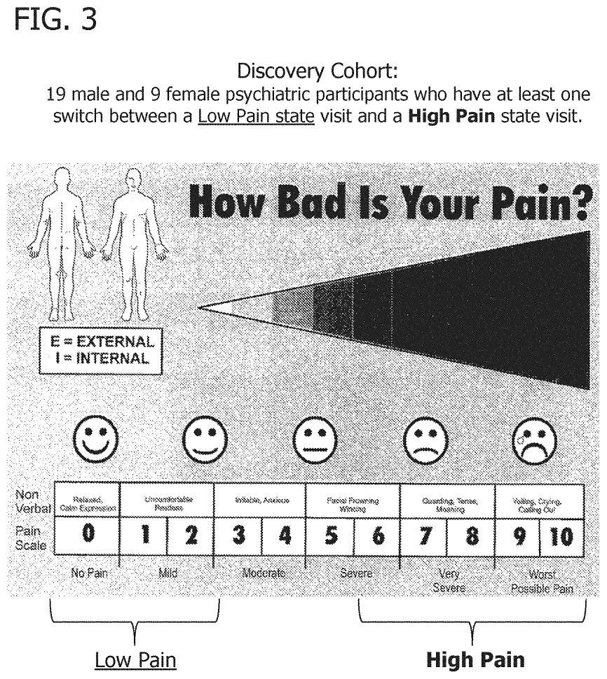

As described herein, “Low Pain” refers to Visual Analog Scale (VAS) for pain of 2 and below; “Intermediate Pain” refers to VAS of 3-5; and “High Pain” refers to VAS of 6 and above (see, FIG. 3). The pain VAS is self-completed by the subject. The pain VAS is a continuous scale comprised of a horizontal (HVAS) or vertical (VVAS) line, usually 10 centimeters (100 mm) in length, anchored by 2 verbal descriptors, one for each symptom extreme (at 0 for “no pain” and at 100 for “worst imaginable pain”). The subject is asked to place a line perpendicular to the VAS line at the point that represents their pain intensity. Using a ruler, the score (i.e., intensity of pain) is determined by measuring the distance (mm) on the 10-cm line between the “no pain” anchor and the patient's mark, providing a range of scores from 0-100. A higher score indicates greater pain intensity.

While not used herein, other suitable pain tests include, for example, numeric rating scale (NRS), McGill Pain Questionnaire (MPQ), Short-form McGill Pain Questionnaire (SF-MPQ), Chronis Pain Grade Scale (CPGS), Short form 36 Bodily Pain Scale (SF-36 BPS), Measure of Intermittent and Constant Osteoarthritis Pain (ICOAP), and combinations thereof. For more information on these tests and applications thereof, see Hawker et al., Arthritis Care & Research, vol. 36, no. S11, November 2011, pp. S240-S252.

The biomarkers are selected from the group listed in Table 1, 4, 5, 7 and combinations thereof. In some embodiments, a panel of blood biomarkers is used. Biomarkers can be selected with different weighting coefficients possible.

In some embodiments, the expression level of the blood biomarker in the sample obtained from the subject is increased as compared to the reference expression level of the biomarker.

In some embodiments, the expression level of the blood biomarker in the sample obtained from the subject is decreased as compared to the reference expression level of the biomarker.

A particularly suitable biomarker for determining pain intensity is CNTN1.

In some embodiments, the subject is a female. A particularly suitable biomarker for predicting pain state in female subjects is DNAJC18.

In some embodiments, the subject is male. A particularly suitable biomarker for predicting pain state in female subjects is CTN1.

In some embodiments, the method further includes performing a neuropsychological test on the subject.

Methods for Predicting Future Medical Care Facility Visit for Pain

In another aspect, the present disclosure is directed to a method for predicting a future medical care facility visit for pain in a subject in need thereof. The method includes: obtaining an expression level of a blood biomarker in a sample obtained from the subject; obtaining a reference expression level of the blood biomarker; and identifying a difference in the expression level of the blood biomarker in the sample and the reference expression level of the blood biomarker, whereas the difference in the expression level of the blood biomarker in the sample obtained from the subject and the reference expression level of the blood biomarker determines the likelihood of future medical care facility/emergency department (ED) visits for pain.

As used herein, “emergency department (ED)” is used according to its ordinary meaning as understood by those skilled in the art to refer to medical care facilities specializing in emergency medicine, the acute care of patients who present without prior appointment; either by their own means or by that of an ambulance, and includes accident & emergency departments (A&E), emergency rooms (ER), emergency wards (EW) and casualty departments.

The biomarker is selected from the group listed in Table 1, 4, 5, 7 and combinations thereof. In some embodiments, a panel of blood biomarkers is used. Biomarkers can be selected with different weighting coefficients possible.

In some embodiments, the expression level of the blood biomarker in the sample obtained from the subject is increased as compared to the reference expression level of the biomarker.

In some embodiments, the expression level of the blood biomarker in the sample obtained from the subject is decreased as compared to the reference expression level of the biomarker.

GBP1 is particularly suitable for predicting trait first year ED visits. GNG7 is particularly suitable for predicting trait all future ED visits.

In some embodiments, the subject is a female. GBP1 is particularly suitable as a predictor for trait first year ED visits in female subjects. ASTN2 is particularly suitable for trait all future ED visits in female subjects. When the subject is a female with bipolar disorder, CDK6 is a particularly suitable predictor for state. When the subject is a female with PTSD, SHMT1 is a particularly suitable predictor for trait first year ED visits. When the subject is a female with depression, GNG7 is a particularly suitable for trait all future ED visits.

In some embodiments, the subject is a male. CTN1 is particularly suitable as a predictor for state in male subjects. Hs.554262 is particularly suitable as a predictor for trait first year ED visits in male subjects. MFAP3 is particularly suitable for trait all future ED visits in male subjects. When the subject is a male with depression, CASPS is particularly suitable as a predictor for state. When the subject is a male with PTSD, LY9 is particularly suitable as a strong predictor for trait first year ED visits. When the subject is a male with PTSD MFAP3 is particularly suitable as a strong predictor for trait all future ED visits.

Particularly suitable biomarkers for pain include CCDC144B (Coiled-Coil Domain Containing 144B), COL2A1 (Collagen Type II Alpha 1 Chain), PPFIBP2 (PPFIA Binding Protein 2), DENND1B (DENN Domain Containing 1B), ZNF441 (Zinc Finger Protein 441), TOP3A (Topoisomerase (DNA) III Alpha), and ZNF429 (Zinc Finger Protein 429), and combinations thereof.

In some embodiments, the method further includes performing a neuropsychological test on the subject.

Prognosis of Pain

In another aspect, the present disclosure is directed to a method of prognosing pain in an individual in need thereof. As used herein, the term “prognosing” and “prognosis” are used according to their ordinary meaning as understood by those skilled in the art to refer to pain level increases from no pain to Low Pain to Moderate (Intermediate) Pain to High Pain.

The method includes: obtaining an expression level of a blood biomarker in a sample obtained from the subject; obtaining a reference expression level of the blood biomarker; and identifying a difference in the expression level of the blood biomarker in the sample and the reference expression level of the blood biomarker.

In some embodiments, the method further includes performing a neuropsychological test on the subject.

### Examples

Materials and Methods

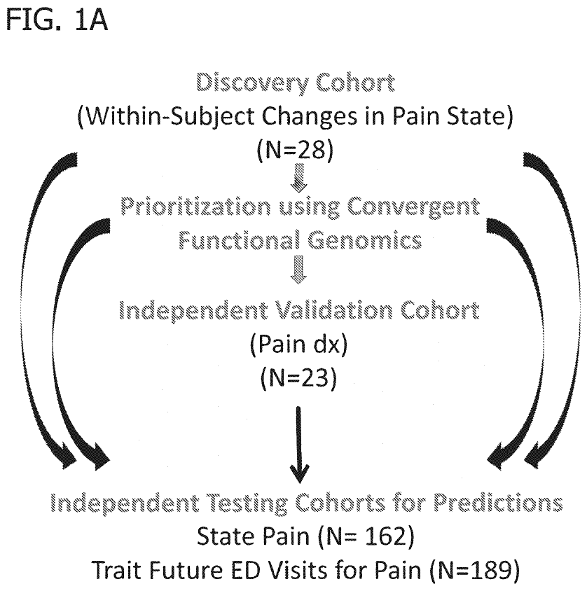

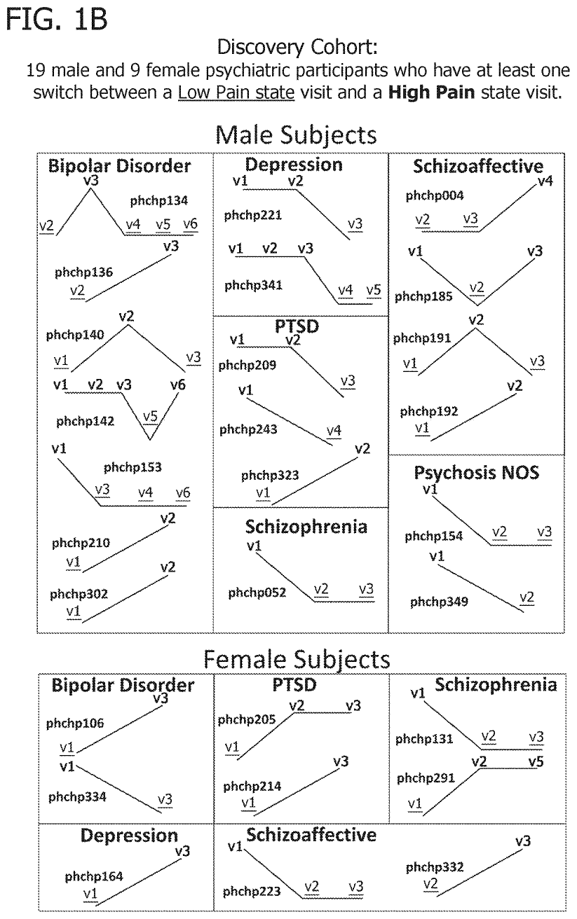

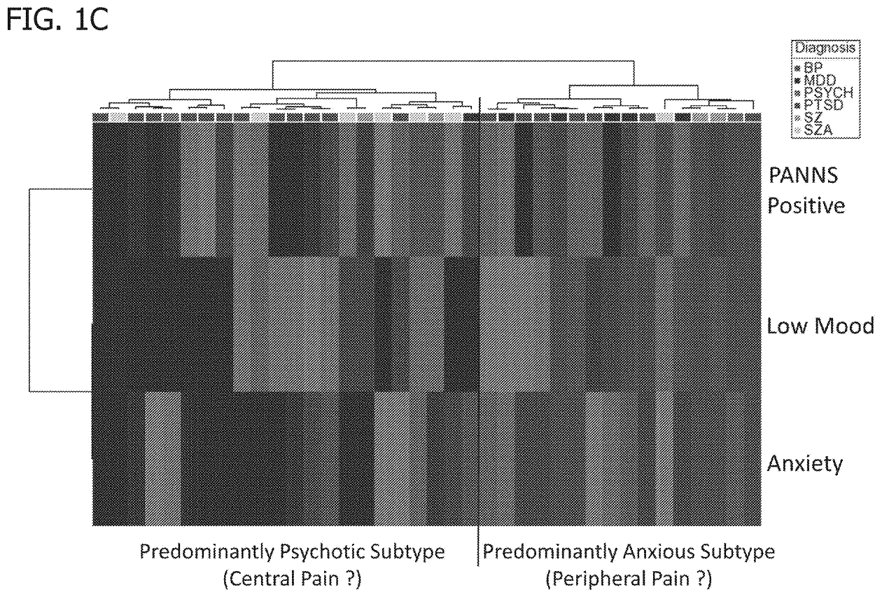

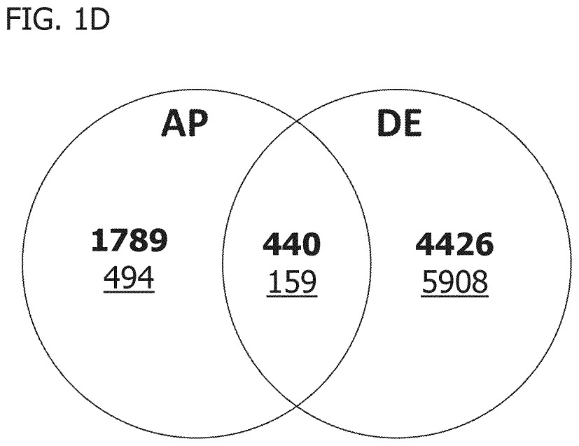

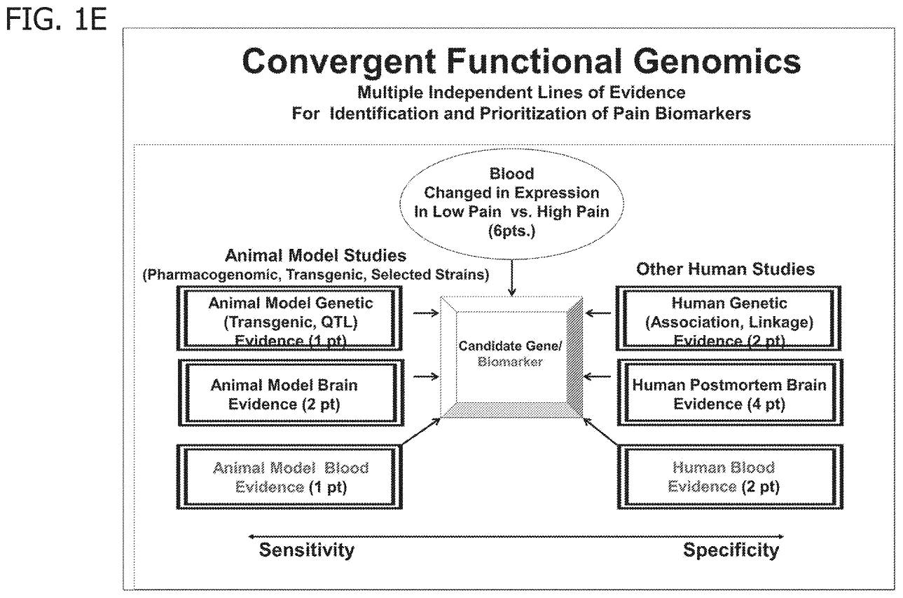

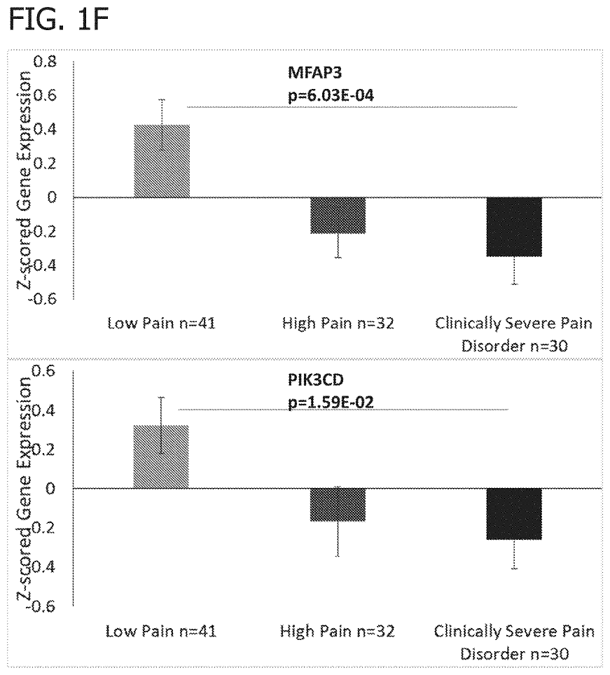

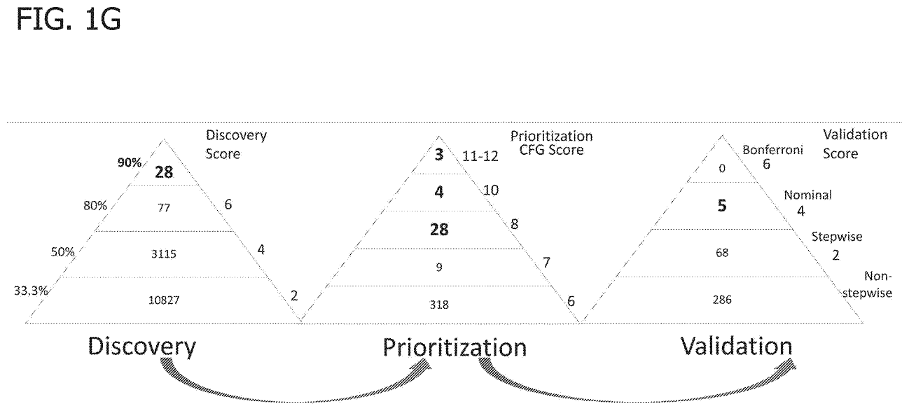

Three independent cohorts were used: discovery (major psychiatric disorders), validation (major psychiatric disorders with clinically severe pain disorders), and testing (an independent major psychiatric disorders cohort for predicting pain state, and for predicting future ER visits for pain) (see, FIG. 1A)

The psychiatric participants/subjects were part of a larger longitudinal cohort of adults that are being continuously collected. Participants were recruited from the patient population at the Indianapolis VA Medical Center. All participants understood and signed informed consent forms detailing the research goals, procedure, caveats and safeguards, per IRB approved protocol. Participants completed diagnostic assessments by an extensive structured clinical interview—Diagnostic Interview for Genetic Studies, and up to six testing visits, 3-6 months apart or whenever a new psychiatric hospitalization occurred. At each testing visit, the subject received a series of rating scales, including a visual analog scale (1-10) for assessing pain and the SF-36 quality of life scale, which has two pain related items (items 21 and 22), and blood was drawn. Whole blood (10 ml) was collected in two RNA-stabilizing PAXgene tubes, labeled with an anonymized ID number, and stored at −80° C. in a locked freezer until the time of future processing. Whole-blood RNA was extracted for microarray gene expression studies from the PAXgene tubes, as detailed below.

For these Examples, the within-participant discovery cohort, from which the biomarker data were derived, consisted of 28 participants (19 males, 9 females) with multiple testing visits, who each had at least one diametric change in pain from Low Pain (VAS of 2 and below) to High Pain (VAS of 6 and above) from one testing visit to another (FIGS. 1B and 3). There were 3 participants with 5 visits each, 1 participants with 4 visits each, 12 participants with 3 visits each, and 12 participants with 2 visits each resulting in a total of 79 blood samples for subsequent gene expression microarray studies (FIGS. 1A-1C; Table 3).

The validation cohort, in which the top biomarker findings were validated for being even more changed in expression, consisted of 13 male and 10 female participants with a pain disorder diagnosis and clinically severe pain (Table 3). This was determined as having a pain VAS of 6 and above and a sum of SF36 scale items 21 (pain intensity) and 22 (impairment by pain of daily activities) of 10 and above. (See, Table 3).

The independent test cohort for predicting state (High Pain) consisted of 134 male and 28 female participants with psychiatric disorders, demographically matched with the discovery cohort, with one or multiple testing visits, with either Low Pain, intermediate Pain, or High Pain, resulting in a total of 414 blood samples in which whole-genome blood gene expression data were obtained (FIGS. 1A-1C and Table 3).

The test cohort for predicting trait (future ED visits with pain as the primary reason in the first year of follow-up, and all future ED visits for pain) (FIGS. 1A-1C) consisted of 171 males and 19 female participants for which longitudinal follow-up with electronic medical records were obtained. The participants' subsequent number of ED pain-related visits in the year following testing was tabulated from electronic medical records by a clinical researcher, who used the key word “pain” in the reasons for ED visit, or “ache” with a mention of acute pain in the text of the note.

Medications. The participants in the discovery cohort were all diagnosed with various psychiatric disorders, and had various medical co-morbidities (Table 1). Their medications were listed in their electronic medical records, and documented at the time of each testing visit. Medications can have a strong influence on gene expression. However, the discovery of differentially expressed genes was based on within-participant analyses, which factored out not only genetic background effects, but also minimizes medication effects, as the participants rarely had major medication changes between visits. Moreover, there was no consistent pattern of any particular type of medication, as the participants were on a wide variety of different medications, psychiatric and non-psychiatric. Some participants may be non-compliant with their treatment and may thus have changes in medications or drug of abuse not reflected in their medical records. That being said, the goal was to discover biomarkers that track pain, regardless if the reason for it was endogenous biology or driven by substance abuse or medication non-compliance. In fact, one would expect some of these biomarkers to be targets of medications. Overall, the discovery of biomarkers with the universal design occurred despite the participants having different genders, diagnoses, being on various different medications, and other lifestyle variables.

Blood Gene Expression Experiments

RNA extraction. Whole blood (2.5-5 ml) was collected into each PaxGene tube by routine venipuncture. RNA was extracted and processed as previously described (see, Le-Niculescu, H. et al. Mol Psychiatry 18, 1249-64 (2013); Niculescu, A. B. et al. Mol Psychiatry 20, 1266-85 (2015); Levey, D. F. et al. Mol Psychiatry 21, 768-85 (2016)).

Microarrays. Microarray work was carried out as previously described (see, Le-Niculescu, H. et al. Mol Psychiatry 18, 1249-64 (2013); Niculescu, A. B. et al. Mol Psychiatry 20, 1266-85 (2015); Levey, D. F. et al. Mol Psychiatry 21, 768-85 (2016)).

Biomarkers

Step 1: Discovery.

The participant's score from the VAS Pain Scale was used, assessed at the time of blood collection (FIGS. 1A-1C). Gene expression differences between visits were analyzed with Low Pain (defined as a score of 0-2) and visits with High Pain (defined as a score of 6 and above), using a powerful within-participant design, then an across-participants summation (FIGS. 1A-1C).

Data was analyzed using an Absent-Present (AP) approach and a differential expression (DE) approach (see, Le-Niculescu, H. et al. Mol Psychiatry 18, 1249-64 (2013); Niculescu, A. B. et al. Mol Psychiatry 20, 1266-85 (2015); Levey, D. F. et al. Mol Psychiatry 21, 768-85 (2016)). The AP approach can capture turning on and off of genes, and the DE approach can capture gradual changes in expression. R scripts were developed to automate and conduct all these large dataset analyses in bulk, checked against human manual scoring.

Gene symbol for the probe sets were identified using NetAffyx (Affymetrix) for Affymetrix HG-U133 Plus 2.0 GeneChips, followed by GeneCards to confirm the primary gene symbol. For those probesets that were not assigned a gene symbol by NettAffyx, GeneAnnot was used to obtain gene symbols for the uncharacterized probesets, followed by GeneCard. Genes were then scored using a manually curated CFG database as described below (FIG. 1E).

Step 2. Prioritization using Convergent Functional Genomics (CFG).

Databases. Manually curated databases of the human gene expression/protein expression studies (postmortem brain, peripheral tissue/fluids: CSF, blood and cell cultures), human genetic studies (association, copy number variations and linkage), and animal model gene expression and genetic studies, published to date on psychiatric disorders, were created. Only findings deemed significant in the primary publication, by the study authors, using their particular experimental design and thresholds were included in the databases. The databases included only primary literature data and did not include review papers or other secondary data integration analyses to avoid redundancy and circularity. These large and constantly updated databases have been used in the inventors' CFG cross validation and prioritization platform (FIG. 1E). For these Examples, data from 355 papers on pain were present in the databases at the time of the CFG analyses (December 2017) (human genetic studies-212, human nervous tissue studies-3, human peripheral tissue/fluids-57, non-human genetic studies-26, non-human brain/nervous tissue studies-48, non-human peripheral tissue/fluids-9). Analyses were performed as described herein and in Le-Niculescu, H. et al. Mol Psychiatry 18, 1249-64 (2013); Niculescu, A. B. et al. Mol Psychiatry 20, 1266-85 (2015); Levey, D. F. et al. Mol Psychiatry 21, 768-85 (2016).

Step 3. Validation analysis.

Validation analyses of candidate biomarker genes were conducted separately for AP and for DE. Which of the top candidate genes (total CFG score of 6 or above), were stepwise changed in expression from the Low Pain and High Pain group to the Clinically Severe Pain group was determined. A CFG score of 6 or above reflected an empirical cutoff of 33.3% of the maximum possible CFG score of 12, which permitted the inclusion of potentially novel genes with maximal internal score of 6 but no external evidence score. Participants with Low Pain, as well as participants with High Pain from the discovery cohort who did not have severe clinical pain (SF36 sum of item 21 and 22<10) were used, along with the independent validation cohort which all had severe clinical pain and a co-morbid pain disorder diagnosis (n=23).

For the AP analysis, the Affymetrix microarray .chp data files from the participants in the validation cohort of severe pain were imported into MASS Affymetrix Expression Console, alongside the data files from the Low Pain and High Pain groups in the live discovery cohort. The AP data was transferred to an Excel sheet and A was transformed into 0, M into 0.5 and P into 1. Everything was Z-scored together by gender and diagnosis. If a probe set would have shown no variance, and thus, gave a non-determined (0/0) value in Z-scoring in a gender and diagnosed, the value was excluded from the analysis for that probeset for that gender and diagnosis from the analysis.

For the DE analysis, the cohorts were assembled out of Affymetrix .cel data that was RMA normalized by gender and diagnosis. The log transformed expression data was transferred to an Excel sheet, and non-log data transformed by taking 2 to the power of the transformed expression value. The values were then Z-scored by gender and diagnosis.

The Excel sheets with the Z-scored by gender and diagnosis AP and DE expression data were imported into Partek, and statistical analyses were performed using a one-way ANOVA for the stepwise changed probesets, and a stringent Bonferroni corrections were performed for all the probesets tested in AP and DE (stepwise and non-stepwise) (FIG. 1F). An R script that automatically analyzes the data directly from the Excel sheet was then developed and used to confirm the calculations.

Choice of Biomarkers to be Carried Forward

The top biomarkers from each step were carried forward. The longer list of candidate biomarkers includes the top biomarkers from discovery step (>=90% of scores, n=28), the top biomarkers from the prioritization step (CFG score>=8, n=32), and the nominally significant biomarkers after the validation step (n=5), for a total of n=65 probesets (n=60 genes). The short list of top biomarkers after the validation step is 5 biomarkers. In Step 4 testing, prediction with the biomarkers from the long list in independent cohorts High Pain state, and future ED visits for pain in the first year, and in all future years were performed.

### Diagnostics

The test cohort for predicting High Pain (state), and the subset of it that was a test cohort for predicting future ER visits (trait), were assembled out of data that was RMA normalized by gender and diagnosis. The cohort was completely independent, as there was no subject overlap with the discovery cohort. Phenomic (clinical) and gene expression markers used for predictions were Z-scored by gender and diagnosis to be able to combine different markers into panels and to avoid potential artifacts due to different ranges of expression in different gender and diagnoses. Markers were combined by simple summation of the increased risk markers minus the decreased risk markers. Predictions were performed using R studio.

Predicting High Pain State. Receiver-operating characteristic (ROC) analyses between genomic and phenomic marker levels and Pain were performed by assigning participants with a Pain score of 6 and greater into the High Pain category. The pROC package of R (Xavier Robin et al. BMC Bioinformatics 2011) was used. The z-scored biomarker and phene scores were run in the ROC generating program against the diagnostic groups in the independent test cohort (High Pain vs. the rest of participants). Additionally, a one-tailed t-test was performed between High Pain group versus the rest, and Pearson R (one-tail) was calculated between Pain scores and marker levels.

Predicting Future ER visits for Pain in First Year Following Testing. Analysis for predicting ER visits for Pain in the first year following each testing visit in subjects that had at least one year of follow-up in the VA system was conducted. ROC analysis between genomic and phenomic marker levels at specific testing visit and future ER visits for Pain were performed as previously described based on assigning if participants had visited the ER with primary reason for Pain or not within one year following a testing visit. Additionally, a one tailed t-test with unequal variance was performed between groups of participant visits with and without ER visits for pain. Person R (one-tail) correlation was performed between hospitalization frequency (number of ER visits for pain divided by duration of follow-up) and marker levels. A Cox regression was performed using the time in days from the testing visit date to first ER visit date in the case of patients who had been to the ER, or 365 days for those who did not. The hazard ratio was calculated such that a value greater than 1 always indicated increased risk for ER visits, regardless if the biomarker was increased or decreased in expression.

Odds ratio analysis was conducted for ER visits for pain for all future ER visits due to pain, including those occurring beyond one year of follow-up, in the years following testing (on average 5.26 years per participant, range 0.44 to 11.27 years; see Tables 1 and 3), as this calculation, unlike the ROC and t-test, accounts for the actual length of follow-up, which varied from participant to participant. Without being bound by theory, the ROC and t-test may, if used, under-represent the power of the markers to predict, as the more severe psychiatric patients are more likely to move geographically and/or be lost to follow-up. A Cox regression was also performed using the time in days from visit date to first ER Pain visit date in the case of patients who had been to the ER for pain, or from visit date to last note date in the electronic medical records for those who did not. The hazard ration was calculated such that a value greater than 1 always indicated increased risk for ER Pain related visits, regardless if the biomarker was increased or decreased in expression.

Biological Understanding

Pathway Analysis

IPA (Ingenuity Pathway Analysis, version 24390178, Qiagen), David Functional Annotation Bioinformatics Microarray Analysis (National Institute of Allergy and Infectious Diseases) version 6.7 (August 2016), and Kyoto Encyclopedia of Genes and Genomes (KEGG) (through DAVID) were used to analyze the biological roles, including top canonical pathways and diseases (Table 6), of the candidate genes resulting from these Examples, as well as to identify genes in the dataset that were the target of existing drugs. The pathway analysis for the combined AP and DE probesets identified 60 unique genes (65 probesets). Network analysis of the 60 unique genes was performed using STRING Interaction Network by in putting the genes into the search window and performing Multiple Proteins Homo sapiens analysis.

CFG beyond Pain: evidence for involvement in other psychiatric and related disorders.

A CGF approach was also used to examine evidence from other psychiatric and related disorders for the list of 65 candidate biomarkers (Table 5).

Therapeutics

Pharmacogenomics. Which of the individual top biomarkers were analyzed for knowing to be modulated by existing drugs using the CFG databases and using Ingenuity Drugs analysis (Table 7).

New drug discovery/repurposing. Drugs and natural compounds were also analyzed as an opposite match for the gene expression profile of panels of the top biomarkers (n=65) using the Connectivity Map (Broad Institute, MIT) (Table 2). 33 of 65 probesets were present in the HGU-133A array used for the Connectivity Map. The NIH LINCS L1000 database was also used (Table 4).

Convergent Functional Evidence

All the evidence from discovery (up to 6 points), prioritization (up to 12 points), validation (up to 6 points), testing (state, trait first year ED visits, trait all future ED visits-up to 8 points each if significantly predicts in all participants, 6 points if predicts by gender, 4 points if predicts in gender/diagnosis) were tabulated into a convergent functional evidence score. The total score could be up to 48 points: 36 from this data and 12 from literature data. The data from these Examples were weighed three times as much as the literature data. The Examples highlight, based on the totality of the experimental data and of the evidence in the field to date, biomarkers having all around evidence: those that tracked pain, those that predicted it, those that were reflective of pain and other pathology, and those that were potential drug targets.

Provided herein is a powerful longitudinal within-participant design in individuals with psychiatric disorders to discover blood gene expression changes between self-reported Low Pain and High Pain states (FIGS. 1A-1C). A longitudinal within-participant design is orders of magnitude more powerful than a cross-sectional case-control design. Some of these candidate gene expression biomarkers are increased in expression in High Pain states (being putative risk genes, or “algogenes”), and others are decreased in expression (being putative protective genes, or “pain suppressor genes”).

The list of candidate biomarkers was prioritized with a Bayesian-like Convergent Functional Genomics approach, comprehensively integrating previous human and animal model evidence in the field.

The top biomarkers from discovery and prioritization were validated in an independent cohort of psychiatric subjects carrying a diagnosis of a pain disorder and with high scores on pain severity ratings. A list of 65 candidate biomarkers (Tables 1 and 3), including a shorter list of 5 validated biomarkers (MFAP3, PIK3CD, SVEP1, TNFRSF11B, ELAC2) was obtained from the first three steps. The biomarkers with the best evidence after validation were Hs.666804/MFAP3 (p=6.03E-04) and PIK3CD (p=1.59E-02).

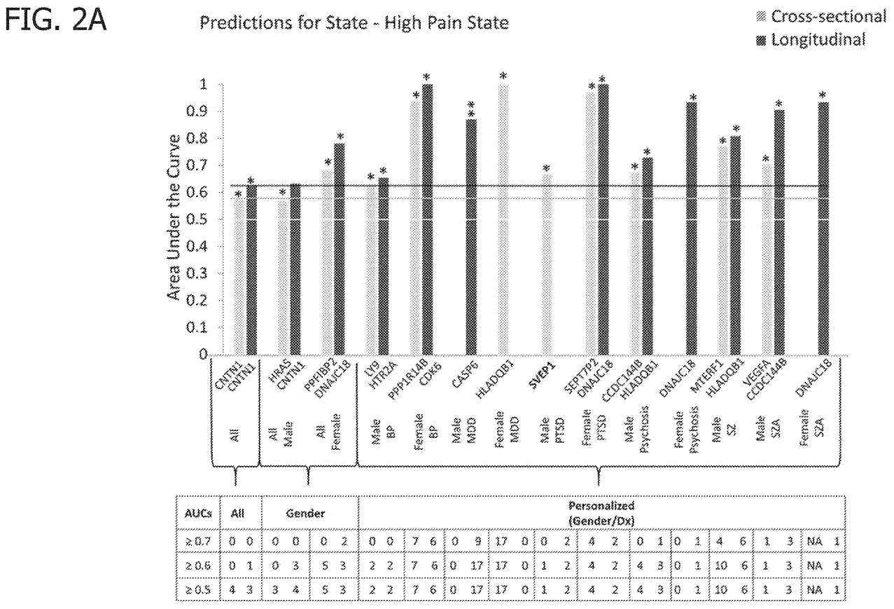

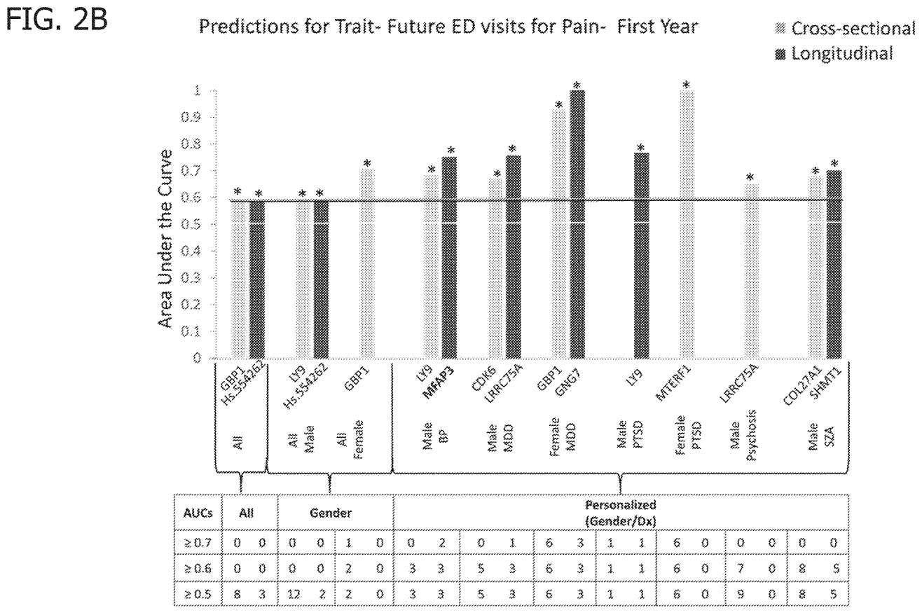

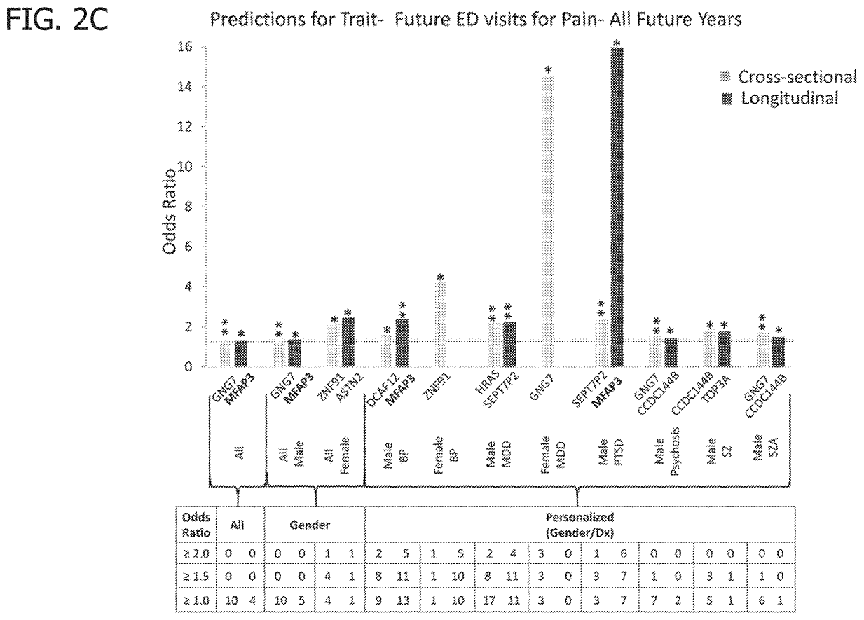

The 65 candidate biomarkers were analyzed for predicting pain severity state and future emergency department (ED) visits for pain in another independent cohort of psychiatric subjects. The biomarkers were analyzed in all subjects in the test cohort, as well as by gender and psychiatric diagnosis, which showed increased accuracy, particularly in women (FIG. 2). In general, the longitudinal information was more predictive than the cross-sectional information. Across all participants tested, CNTN1 was the best predictor for state (AUC 63%, p=0.0014), GBP1 the best predictor for trait first year ED visits (AUC 59%, p=0.0035), and GNG7 the best predictor for trait all future ED visits (OR 1.28, p=0.000161, surviving Bonferroni correction for the 65 biomarkers tested). By gender, in females, DNAJC18 was the best predictor for state (AUC 78%, p=0.0049), GBP1 the best predictor for trait first year ED visits (AUC 71%, p=0.043) and ASTN2 for trait all future ED visits (OR 2.45, p=0.043). In males, CNTN1 was the best predictor for state (AUC 63%, p=0.0022), Hs.554262 the best predictor for trait first year ED visits (AUC 59%, p=0.016), and MFAP3 the best predictor for trait all future ED visits (OR 1.34, p=0.014). Personalized by gender and diagnosis, in female bipolar, CDK6 was a strong predictor for state (AUC 100%, p=0.007), in female PTSD, SHMT1 was a strong predictor for trait first year ED visits (AUC 100%, p=0.022), and in female depression GNG7 for trait all future ED visits (OR 14.54, p=0.023). In male depression, CASPS was a strong predictor for state (AUC 87%, p=0.00007, surviving Bonferroni correction for the 65 biomarkers tested), in male PTSD, LY9 was a strong predictor for trait first year ED visits (AUC 77%, p=0.041), and in male PTSD, MFAP3 was a strong predictor for trait all future ED visits (OR 15.95, p=0.00084). Predictions of future ED visits for pain in the independent cohorts were consistently stronger using biomarkers than clinical phenotypic markers (pain VAS scale, pain items 21 and 22 from SF-36), supporting the utility of biomarkers. Also, in general, panels of all 65 biomarkers or of the 5 validated biomarkers did not work as well as individual biomarkers, particularly when the later are tested by gender and diagnosis, consistent with there being heterogeneity in the population and supporting the need for personalization. The notable exception was predicting all future ED visits for pain, where the panel of 5 validated biomarkers performed better than individual biomarkers.

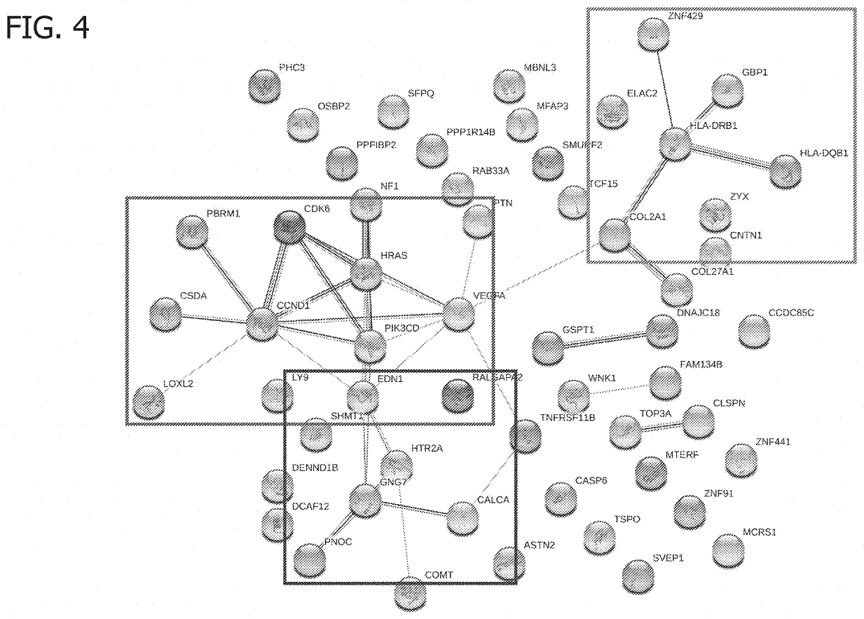

The biomarkers were further analyzed for involvement in other psychiatric and related disorders (Table 5). A majority of the biomarkers have some evidence in other disorders, whereas a few seemed to be specific for pain, such as CCDC144B (Coiled-Coil Domain Containing 144B), COL2A1 (Collagen Type II Alpha 1 Chain), PPFIBP2 (PPFIA Binding Protein 2), DENND1B (DENN Domain Containing 1B), ZNF441 (Zinc Finger Protein 441), TOP3A (Topoisomerase (DNA) III Alpha), and ZNF429 (Zinc Finger Protein 429). A majority of the biomarkers (50 out of 60 genes, i.e. 83.3%) have prior evidence for involvement in suicide, indicating an extensive molecular co-morbidity between pain and suicide, to go along with the clinical and phenomenological co-morbidity (physical pain, psychic pain). The biological pathways and networks the biomarkers are involved in were analyzed (Table 6 and FIG. 4). There was a network centered on GNG7 (FIG. 4), that may be involved in connectivity/signaling, comprising HTR2A, EDN1, PNOC (involved in pain signaling) and CALCA (involved in Reflex Sympathetic Dystrophy and Complex Regional Pain Syndrome). It was reassuring that PNOC (Prepronociceptin) increased in expression in high pain states, i.e. as an algogene. Given its known roles in pain, it can serve as a de facto positive control. A second network was centered on CCND1, may be involved in activity/trophicity, and comprises HRAS, CDK6, PBRM1, CSDA, LOXL2, EDN1, PIK3CD, and VEGFA. A third network was centered on HLA DRB1, may be involved in reactivity/immune response, and comprises GBP1, ZNF429, COL2A1, and HLA DQB1, from the list of 65 top biomarkers.

The biomarkers were analyzed as targets of existing drugs and thus could be used for pharmacogenomics population stratification and measuring of response to treatment (Table 7), as well as used the biomarker gene expression signature to interrogate the Connectivity Map database from Broad/MIT to identify drugs and natural compounds that can be repurposed for treating pain (Table 2). The top drugs identified as potential new pain therapeutic were SC-560, an NSAID, haloperidol, an antipsychotic, and amoxapine, an antidepresseant. The top natural compounds were pyridoxine (vitamin B6), cyanocobalamin (vitamin B12), and apigenin (a plant flavonoid).

The biomarkers with the best overall evidence across the six steps were GNG7, CNTN1, LY9 CCDC144B, GBP1 and MFAP3 (Table 1). GNG7 (G Protein Subunit Gamma 7) was decreased in expression in blood in High Pain states, i.e., it is a pain suppressor gene. There is evidence in other tissues in human studies for involvement in pain (diabetic neuropathy, vertebral disc). GNG7 also has trans-diagnostic evidence for involvement in other psychiatric disorders. It is decreased in expression in mouse brain by alcohol, hallucinogens, and stress, and increased in expression by omega-3 fatty acids. CNTN1 (Contactin 1) was decreased in expression in blood in High Pain states, i.e. it is a pain suppressor gene. Reassuringly, there was convergent evidence in other tissues in human studies for involvement in pain: CNTN1 has also been reported to be decreased in expression in CSF in women with chronic widespread pain (CWP). Anti-contactin 1 autoantibodies, that block/decrease levels of contactin 1, have been described in chronic inflammatory demyelinating polyneuropathy4. CNTN1 has also trans-diagnostic evidence for involvement in psychiatric disorders. It is decreased in expression in schizophrenia brain and blood, and in blood in suicidality in females. CNTN1 was increased in expression by clozapine in mouse brain. LY9 (Lymphocyte Antigen 9) was increased in expression in blood in High Pain states, i.e. it is an algogene. It also has epigenetic evidence for involvement in exposure to stress, and is decreased in expression by omega-3 fatty acids in mouse brain. CCDC144B (Coiled-Coil Domain Containing 144B) was decreased in expression in blood in High Pain states. There is evidence in other tissues in human and animal model studies for involvement in pain. CCDC144B was a good predictor in the independent cohorts for state and trait, particularly for males with psychosis (SZ, SZA). It does not have trans-diagnostic evidence for involvement in other psychiatric disorders, seeming to be relatively specific for pain. GBP1 (Guanylate Binding Protein 1), with interferon induced signaling roles, is increased in expression in blood in High Pain states. There is other evidence in human studies, gene expression and genetic, for involvement in pain. GBP1 is a predictor in the independent cohorts for trait, particularly in females. It is increased in expression in the brain in MDD, schizophrenia, and suicide, and in blood in PTSD. GBP1 was decreased in expression by omega-3 in mouse brain. Hs.666804/MFAP3 (Microfibril Associated Protein 3), another of the top markers, is a component of elastin-associated microfibrils. MFAP3 had the most robust empirical evidence from the discovery and validation steps, and was a strong predictor in the independent cohort, particularly for pain in females and males with PTSD. Interestingly, it has no prior evidence for pain in the literature curated to date for the Prioritization/CFG step, which demonstrates that a wide-enough net was cast with the disclosed approach that can bring to the fore completely novel findings. MFAP3 was decreased in expression in blood in High Pain states, i.e., it is a pain suppressor gene. It also has previous evidence for involvement in alcoholism, stress, and suicide.

As disclosed herein, clustering analysis of a discovery cohort composed of participants with psychiatric disorders followed longitudinally over time, in which each participant had blood samples collected and neuropsychological testing done in at least one low pain state visit (Pain VAS<2 out of 10) and at least one high pain state visit (Pain VAS>6 out of 10), revealed two broad subtypes of high pain states: a predominantly psychotic subtype, possibly related to mis-connectivity and increased perception of pain centrally, and a predominantly anxious subtype, possibly related to reactivity and increased physical health reasons for pain peripherally. The powerful longitudinal within-participant design was used to discover blood gene expression changes between self-reported low pain and high pain states. Some of these gene expression biomarkers were increased in expression in high pain states (being putative risk genes, or “algogenes”), and others were decreased in expression (being putative protective genes, or “pain suppressor genes”).

Advantageously, the present disclosure enables precision medicine for pain, with objective diagnostics and targeted novel therapeutics. Given the massive negative impact of untreated pain on quality of life, the current lack of objective measures to determine appropriateness of treatment, and the severe addiction gateway potential of existing opioid-based pain medications, the present disclosure provides herein. The methods described herein provide objective biomarkers for pain, which is a subjective sensation. Further, the biomarkers provided herein are able to objectively determine pain state and predict future emergency department visits for pain, even more so when personalized by gender and diagnosis. The biomarkers are suitable for targeting using existing drugs and yielded new drug candidates.

In view of the above, it will be seen that the several advantages of the disclosure are achieved and other advantageous results attained. As various changes could be made in the above methods and systems without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

TABLE 1
Convergent Functional Evidence (CFE) for Top Candidate Biomarkers for Pain (n = 60 genes, 65 probesets).

| | | | Step 2 | | Step 4 | Step 4 | Step 4 | | | |
|---|---|---|---|---|---|---|---|---|---|---|
| | | | External | | Best Significant | Best Significant | Best Significant | | | |
| | | | Convergent | | Prediction of | Prediction of Trait- | Predictions of Trait- | | | |
| | | Step 1 | Functional | | State- High | Future ED visits for Pain | Future ED visits for | | | |
| | | Discovery | Genomics | Step 3 | Pain | in the first year | Pain in all future | | Step 6 | CFE |
| | | in Blood | (CFG) | Validation | (Cases/Total) | (Cases/Total) | years | Step 5 | Drugs that | Polyevidence |
| | | (Direction | Evidence For | in Blood | ROC AUC/ | ROC AUC/ | (Cases/Total) | Other | Modulate the | Score for |
| | | of Change) | Involvement | ANOVA p- | p-value | p-value | OR/OR p-value | Psychiatric | Biomarker in | Involvement |
| | | Method/ | in Pain | value/ | 8 pts All | 8 pts All | 8 pts All | and Related | Opposite | in Pain |
| Gene Symbol/ | | Score/% | Score | Score | 6pts Gender | 6pts Gender | 6pts Gender | Disorders | Direction | (Based on |
| Gene Name | Probesets | Up to 6pts | Up to 12pts | Up to 6 pts | 4pts Gender/Dx | 4pts Gender/Dx | 4pts Gender/Dx | Evidence | to Pain | Steps 1-4) |
| GNG7 | 1566643_a_at | (D) | 6 | 6.81E−02/2 | All | Gender | All | Alcohol | Omega-3 | 34 |
| G Protein Subunit | | DE/4 | | Stepwise | C: (101/411) | Females | C: (239/501) | BP | fatty acids | |
| Gamma 7 | | 59% | | | 0.56/3.52E−02 | C: (7/44) | 1.28/1.03E−04** | Hallucinogens | | |
| | | | | | Gender | 0.7/4.92E−02 | L: (145/309) | MDD | | |
| | | | | | Male | Gender/Dx | 1.22/1.70E−02 | Stress | | |
| | | | | | C: (85/346) | F-MDD | Gender | SZ | | |
| | | | | | 0.56/3.95E−02 | C: (4/11) | Females | | | |
| | | | | | Gender/Dx | 0.82/4.45E−02 | C: (13/47) | | | |
| | | | | | M-SZ | L: (2/6) | 1.69/4.69E−02 | | | |
| | | | | | C: (11/64) | 1/3.20E−02 | Males | | | |
| | | | | | 0.68/2.79E−02 | F-PTSD | C: (226/454) | | | |
| | | | | | | C: (2/8) | 1.28/1.92E−04** | | | |
| | | | | | | 0.92/4.78E−02 | L: (138/282) | | | |
| | | | | | | | 1.21/2.16E−02 | | | |
| | | | | | | | Gender/Dx | | | |
| | | | | | | | F-MDD | | | |
| | | | | | | | C: (4/12) | | | |
| | | | | | | | 14.54/2.23E−02 | | | |
| | | | | | | | M-MDD | | | |
| | | | | | | | L: (25/43) | | | |
| | | | | | | | 1.8/2.70E−02 | | | |
| | | | | | | | M-PSYCHOSIS | | | |
| | | | | | | | C: (95/201) | | | |
| | | | | | | | 1.52/1.70E−04** | | | |
| | | | | | | | L: (57/120) | | | |
| | | | | | | | 1.34/2.47E−02 | | | |
| | | | | | | | M-SZ | | | |
| | | | | | | | C: (42/103) | | | |
| | | | | | | | 1.58/2.08E−02 | | | |
| | | | | | | | M-SZA | | | |
| | | | | | | | C: (53/98) | | | |
| | | | | | | | 1.71/4.40E−04** | | | |
| CNTN1 | 1554784_at | (D) | 6 | NS | All | Gender | Gender/Dx | BP | Clozapine | 28 |
| Contactin 1 | | DE/4 | | | C: (101/411) | Males | M-MDD | MDD | | |
| | | 52% | | | 0.58/1.15E−02 | C: (95/426) | C: (42/72) | SZ | | |
| | | | | | L: (61/248) | 0.56/3.08E−02 | 1.44/1.23E−02 | Suicide | | |
| | | | | | 0.63/1.42E−03 | | L: (25/43) | | | |
| | | | | | Gender | | 1.64/4.17E−02 | | | |
| | | | | | Female | | | | | |
| | | | | | C: (16/65) | | | | | |
| | | | | | 0.65/3.38E−02 | | | | | |
| | | | | | Male | | | | | |
| | | | | | L: (51/212) | | | | | |
| | | | | | 0.63/2.27E−03 | | | | | |
| | | | | | Gender/Dx | | | | | |
| | | | | | M-BP | | | | | |
| | | | | | C: (24/123) | | | | | |
| | | | | | 0.61/4.13E−02 | | | | | |
| | | | | | L: (16/81) | | | | | |
| | | | | | 0.64/4.06E−02 | | | | | |
| | | | | | M-SZ | | | | | |
| | | | | | C: (11/64) | | | | | |
| | | | | | 0.68/3.15E−02 | | | | | |
| | | | | | M-MDD | | | | | |
| | | | | | L: (13/43) | | | | | |
| | | | | | 0.66/4.53E−02 | | | | | |
| | | | | | M-SZA | | | | | |
| | | | | | L: (3/17) | | | | | |
| | | | | | 0.83/3.89E−02 | | | | | |
| LY9 | 231124_x_at | (I) | 2 | NS | All | All | Gender/Dx | Acute Stress | Omega-3 | 28 |
| Lymphocyte Antigen | | DE/6 | | | C: (101/411) | C: (102/470) | M-MDD | | fatty acids | |
| 9 | | 90% | | | 0.56/4.40E−02 | 0.56/2.30E−02 | C: (42/72) | | | |
| | | | | | L: (61/248) | Gender | 1.65/3.85E−03 | | | |
| | | | | | 0.58/2.39E−02 | Males | L: (25/43) | | | |
| | | | | | Gender | C: (95/426) | 1.53/3.74E−02 | | | |
| | | | | | Male | 0.59/2.61E−03 | M-PTSD | | | |
| | | | | | C: (85/346) | Gender/Dx | L: (18/20) | | | |
| | | | | | 0.57/3.02E−02 | M-BP | 2.07/6.77E−03 | | | |
| | | | | | L: (51/212) | C: (18/120) | | | | |
| | | | | | 0.62/5.19E−03 | 0.68/6.91E−03 | | | | |
| | | | | | Gender/Dx | M-PTSD | | | | |
| | | | | | M-BP | L: (10/16) | | | | |
| | | | | | C: (24/123) | 0.77/4.13E−02 | | | | |
| | | | | | 0.63/2.66E−02 | | | | | |
| | | | | | F-MDD | | | | | |
| | | | | | C: (2/18) | | | | | |
| | | | | | 0.97/1.75E−02 | | | | | |
| | | | | | M-MDD | | | | | |
| | | | | | L: (13/43) | | | | | |
| | | | | | 0.8/9.87E−04 | | | | | |
| CCDC144B | 1557366_at | (D) | 6 | NS | Gender/Dx | Gender/Dx | All | | | 26 |
| Coiled-Coil Domain | | DE/4 | | | F-BP | M-MDD | C: (239/501) | | | |
| Containing 144B | | 56% | | | C: (4/21) | C: (26/67) | 1.23/2.27E−03 | | | |
| (Pseudogene) | | | | | 0.79/3.66E−02 | 0.63/3.43E−02 | Gender | | | |
| | | | | | M-PSYCHOSIS | | Males | | | |
| | | | | | C: (19/96) | | C: (226/454) | | | |
| | | | | | 0.68/8.95E−03 | | 1.23/3.34E−03 | | | |
| | | | | | L: (10/56) | | Gender/Dx | | | |
| | | | | | 0.68/4.16E−02 | | M-PSYCHOSIS | | | |
| | | | | | M-SZA | | C: (95/201) | | | |
| | | | | | L: (3/17) | | 1.41/3.46E−03 | | | |
| | | | | | 0.9/1.61E−02 | | L: (57/120) | | | |
| | | | | | | | 1.43/1.32E−02 | | | |
| | | | | | | | M-SZ | | | |
| | | | | | | | C: (42/103) | | | |
| | | | | | | | 1.84/4.65E−03 | | | |
| | | | | | | | M-SZA | | | |
| | | | | | | | L: (32/56) | | | |
| | | | | | | | 1.47/3.49E−02 | | | |
| GBP1 | 231578_at | (I) | 6 | 3.26E−01/2 | | All | All | MDD | Omega-3 | 26 |
| Guanylate Binding | | DE/2 | | Stepwise | | C: (102/470) | C: (239/501) | PTSD | fatty acids | |
| Protein 1 | | 37% | | | | 0.59/3.51E−03 | 1.09/3.72E−02 | SZ | | |
| | | | | | | Gender | Gender | | | |
| | | | | | | Females | Females | | | |
| | | | | | | C: (7/44) | C: (13/47) | | | |
| | | | | | | 0.71/4.30E−02 | 1.68/2.41E−02 | | | |
| | | | | | | Males | Gender/Dx | | | |
| | | | | | | C: (95/426) | F-MDD | | | |
| | | | | | | 0.58/1.04E−02 | C: (4/12) | | | |
| | | | | | | Gender/Dx | 3.1/4.43E−02 | | | |
| | | | | | | F-MDD | M-SZA | | | |
| | | | | | | C: (4/11) | C: (53/98) | | | |
| | | | | | | 0.93/1.17E−02 | 1.22/3.65E−02 | | | |
| | | | | | | M-PSYCHOSIS | | | | |
| | | | | | | C: (33/198) | | | | |
| | | | | | | 0.6/3.25E−02 | | | | |
| | | | | | | M-SZA | | | | |
| | | | | | | C: (23/97) | | | | |
| | | | | | | 0.62/4.10E−02 | | | | |
| Hs.666804/ | 240949_x_at | (D) | 0 | 6.03E−04/4 | Gender/Dx | Gender/Dx | All | Alcohol | | 26 |
| MFAP3 | | DE/6 | | Nominal | F-PTSD | M-BP | L: (145/309) | Suicide | | |
| Microfibril Associated | | 81% | | | C: (5/12) | L: (9/80) | 1.28/2.28E−02 | Stress | | |
| Protein 3 | | | | | 0.8/4.41E−02 | 0.75/7.27E−03 | Gender | | | |
| | | | | | | | Males | | | |
| | | | | | | | C: (226/454) | | | |
| | | | | | | | 1.17/2.64E−02 | | | |
| | | | | | | | L: (138/282) | | | |
| | | | | | | | 1.35/8.94E−03 | | | |
| | | | | | | | Gender/Dx | | | |
| | | | | | | | M-BP | | | |
| | | | | | | | L: (34/91) | | | |
| | | | | | | | 2.36/4.86E−04** | | | |
| | | | | | | | M-PTSD | | | |
| | | | | | | | L: (18/20) | | | |
| | | | | | | | 15.93/8.46E−04 | | | |
| CASP6 | 209790_s_at | (I) | 4 | NS | Gender | Gender | Gender/Dx | BP | | 24 |
| Caspase 6 | | DE/4 | | | Male | Males | M-MDD | | | |
| | | 51% | | | L: (51/212) | C: (95/426) | C: (42/72) | | | |
| | | | | | 0.59/2.92E−02 | 0.57/2.54E−02 | 1.31/3.97E−02 | | | |
| | | | | | Gender/Dx | Gender/Dx | | | | |
| | | | | | F-MDD | M-PSYCHOSIS | | | | |
| | | | | | C: (2/18) | C: (33/198) | | | | |
| | | | | | 1/1.23E−02 | 0.6/2.88E−02 | | | | |
| | | | | | M-MDD | M-SZA | | | | |
| | | | | | L: (13/43) | C: (23/97) | | | | |
| | | | | | 0.87/7.01E−05** | 0.63/2.71E−02 | | | | |
| COMT | 216204_at | (D) | 4 | NS | Gender/Dx | All | Gender/Dx | ADHD | Clozapine | 24 |
| Catechol-O- | | DE/4 | | | M-MDD | C: (102/470) | M-BP | Aggression | Morphine | |
| Methyltransferase | | 54% | | | L: (13/43) | 0.55/4.48E−02 | L: (34/91) | Alcohol | Mood | |
| | | | | | 0.71/1.41E−02 | Gender | 1.65/2.20E−02 | Anxiety | Stabilizers | |
| | | | | | | Males | | BP | | |
| | | | | | | C: (95/426) | | Chronic | | |
| | | | | | | 0.57/1.95E−02 | | Stress | | |
| | | | | | | Gender/Dx | | MDD | | |
| | | | | | | M-MDD | | OCD | | |
| | | | | | | C: (26/67) | | Panic | | |
| | | | | | | 0.66/1.58E−02 | | Disorder | | |
| | | | | | | M-PSYCHOSIS | | Psychosis | | |
| | | | | | | C: (33/198) | | PTSD | | |
| | | | | | | 0.6/3.63E−02 | | Suicide | | |
| | | | | | | | | SZ | | |
| RAB33A | 206039_at | (I) | 0 | NS | Gender/Dx | Gender | All | Alcohol | | 24 |
| RAB33A, Member | | DE/6 | | | F-MDD | Males | C: (239/501) | Stress | | |
| RAS Oncogene Family | | 90% | | | C: (2/18) | C: (95/426) | 1.14/2.21E−02 | MDD | | |
| | | | | | 1/1.23E−02 | 0.56/3.60E−02 | Gender | | | |
| | | | | | | | Males | | | |
| | | | | | | | C: (226/454) | | | |
| | | | | | | | 1.16/1.01E−02 | | | |
| | | | | | | | Gender/Dx | | | |
| | | | | | | | M-BP | | | |
| | | | | | | | L: (34/91) | | | |
| | | | | | | | 1.65/1.69E−03 | | | |
| | | | | | | | M-MDD | | | |
| | | | | | | | C: (42/72) | | | |
| | | | | | | | 1.95/6.59E−04** | | | |
| | | | | | | | L: (25/43) | | | |
| | | | | | | | 1.85/1.72E−02 | | | |
| ZYX | 238016_s_at | (D) | 4 | NS | Gender/Dx | All | Gender/Dx | MDD | Clozapine | 24 |
| Zyxin | | DE/4 | | | F-BP | C: (102/470) | M-BP | | | |
| | | 57% | | | C: (4/21) | 0.55/4.80E−02 | L: (34/91) | | | |
| | | | | | 0.78/4.44E−02 | Gender | 1.85/1.67E−02 | | | |
| | | | | | | Males | M-PTSD | | | |
| | | | | | | C: (95/426) | C: (26/31) | | | |
| | | | | | | 0.57/1.58E−02 | 1.57/4.40E−02 | | | |
| | | | | | | Gender/Dx | L: (18/20) | | | |
| | | | | | | M-PSYCHOSIS | 2.2/1.53E−02 | | | |
| | | | | | | C: (33/198) | | | | |
| | | | | | | 0.62/1.43E−02 | | | | |
| | | | | | | M-SZA | | | | |
| | | | | | | C: (23/97) | | | | |
| | | | | | | 0.66/1.15E−02 | | | | |
| | | | | | | M-BP | | | | |
| | | | | | | L: (9/80) | | | | |
| | | | | | | 0.71/2.26E−02 | | | | |
| (Hs.696420) | 243125_x_at | (D) | 0 | NS | Gender/Dx | Gender/Dx | All | PTSD | | 22 |
| MTERF1 | | DE/6 | | | M-PSYCHOSIS | F-PTSD | C: (239/501) | Suicide | | |
| Mitochondrial | | 100% | | | C: (19/96) | C: (2/8) | 1.19/1.19E−02 | | | |
| Transcription | | | | | 0.67/1.01E−02 | 1/2.28E−02 | L: (145/309) | | | |
| Termination Factor 1 | | | | | M-SZ | | 1.2/4.81E−02 | | | |
| | | | | | C: (11/64) | | Gender | | | |
| | | | | | 0.77/2.27E−03 | | Males | | | |
| | | | | | L: (7/39) | | C: (226/454) | | | |
| | | | | | 0.71/3.95E−02 | | 1.19/1.51E−02 | | | |
| | | | | | | | Gender/Dx | | | |
| | | | | | | | M-PSYCHOSIS | | | |
| | | | | | | | C: (95/201) | | | |
| | | | | | | | 1.41/8.86E−03 | | | |
| | | | | | | | M-SZ | | | |
| | | | | | | | C: (42/103) | | | |
| | | | | | | | 1.4/4.47E−02 | | | |
| | | | | | | | M-SZA | | | |
| | | | | | | | C: (53/98) | | | |
| | | | | | | | 1.44/4.72E−02 | | | |
| COL27A1 | 225293_at | (D) | 4 | 7.47E−01/2 | Gender/Dx | Gender/Dx | Gender/Dx | Tourette | Lithium | 22 |
| Collagen Type XXVII | | DE/4 | | Stepwise | M-MDD | M-MDD | M-PTSD | syndrome | | |
| Alpha 1 Chain | | 79% | | | L: (13/43) | C: (26/67) | L: (18/20) | | | |
| | | | | | 0.66/4.79E−02 | 0.63/3.38E−02 | 1.96/2.37E−02 | | | |
| | | | | | | M-PSYCHOSIS | | | | |
| | | | | | | C: (33/198) | | | | |
| | | | | | | 0.61/2.79E−02 | | | | |
| | | | | | | M-SZA | | | | |
| | | | | | | C: (23/97) | | | | |
| | | | | | | 0.68/4.96E−03 | | | | |
| | | | | | | L: (13/55) | | | | |
| | | | | | | 0.7/1.62E−02 | | | | |
| HRAS | 212983_at | (I) | 0 | NS | All | Gender/Dx | Gender/Dx | Alcohol | ISIS 2503 | 22 |
| HRas Proto- | | DE/6 | | | C: (101/411) | F-PTSD | M-MDD | BP | | |
| Oncogene, GTPase | | 97% | | | 0.56/3.47E−02 | C: (2/8) | C: (42/72) | Longevity | | |
| | | | | | L: (61/248) | 1/2.28E−02 | 2.2/3.38E−06** | Suicide | | |
| | | | | | 0.58/3.01E−02 | | L: (25/43) | SZ | | |
| | | | | | Gender | | 2.25/2.61E−04** | | | |
| | | | | | Male | | | | | |
| | | | | | C: (85/346) | | | | | |
| | | | | | 0.57/2.72E−02 | | | | | |
| | | | | | L: (51/212) | | | | | |
| | | | | | 0.61/1.18E−02 | | | | | |
| | | | | | Gender/Dx | | | | | |
| | | | | | M-SZ | | | | | |
| | | | | | C: (11/64) | | | | | |
| | | | | | 0.68/2.79E−02 | | | | | |
| | | | | | M-MDD | | | | | |
| | | | | | L: (13/43) | | | | | |
| | | | | | 0.71/1.61E−02 | | | | | |
| CALCA | 210727_at | (D) | 7 | NS | Gender | Gender/Dx | | Alcohol | Omega-3 | 21 |
| Calcitonin Related | | DE/4 | | | Females | F-PTSD | | Anxiety | fatty acids | |
| Polypeptide Alpha | | 54% | | | C: (16/63) | C: (2/8) | | Panic | Lithium | |
| | | | | | 0.66/3.12E−02 | 1/2.28E−02 | | Disorder | | |
| | | | | | Gender/Dx | Gender/Dx | | | | |
| | | | | | F-MDD | M-PSYCHOSIS | | | | |
| | | | | | C: (2/18) | C: (33/198) | | | | |
| | | | | | 0.97/1.75E−02 | 0.6/3.87E−02 | | | | |
| | | | | | F-BP | | | | | |
| | | | | | L: (3/11) | | | | | |
| | | | | | 0.88/3.31E−02 | | | | | |
| | | | | | M-MDD | | | | | |
| | | | | | L: (13/43) | | | | | |
| | | | | | 0.66/4.79E−02 | | | | | |
| (Hs.596713) | 226138_s_at | (D) | 0 | 6.28E−02/2 | Gender/Dx | | All | SZ | Lithium | 20 |
| PPP1R14B | | DE/6 | | Stepwise | F-BP | | C: (239/501) | | | |
| Protein Phosphatase | | 90% | | | C: (4/21) | | 1.15/1.43E−02 | | | |
| 1 Regulatory Inhibitor | | | | | 0.94/3.61E−03 | | Gender | | | |
| Subunit 14B | | | | | L: (3/11) | | Males | | | |
| | | | | | 0.92/2.06E−02 | | C: (226/454) | | | |
| | | | | | M-MDD | | 1.19/4.84E−03 | | | |
| | | | | | L: (13/43) | | L: (138/282) | | | |
| | | | | | 0.73/9.98E−03 | | 1.2/3.94E−02 | | | |
| | | | | | | | Gender/Dx | | | |
| | | | | | | | M-PSYCHOSIS | | | |
| | | | | | | | C: (95/201) | | | |
| | | | | | | | 1.35/3.06E−03 | | | |
| | | | | | | | M-SZ | | | |
| | | | | | | | C: (42/103) | | | |
| | | | | | | | 1.53/3.19E−02 | | | |
| | | | | | | | M-SZA | | | |
| | | | | | | | C: (53/98) | | | |
| | | | | | | | 1.41/9.26E−03 | | | |
| ASTN2 | 1554816_at | (I) | 2 | 1.71E−01/2 | | Gender/Dx | Gender | Suicide | Antipsychotics | 20 |
| Astrotactin 2 | | DE/6 | | Stepwise | | F-MDD | Female | SZ | | |
| | | 83% | | | | L: (2/6) | L: (7/27) | ASD | | |
| | | | | | | 1/3.20E−02 | 2.45/4.36E−02 | BP | | |
| | | | | | | | | MDD | | |
| ELAC2 | 201766_at | (D) | 2 | 4.11E−02/4 | Gender/Dx | | Gender | ASD | | 20 |
| ElaC Ribonuclease Z 2 | | DE/4 | | Nominal | M-MDD | | Males | | | |
| | | 52% | | | L: (13/43) | | L: (138/282) | | | |
| | | | | | 0.73/8.66E−03 | | 1.2/4.61E−02 | | | |
| | | | | | | | Gender/Dx | | | |
| | | | | | | | M-BP | | | |
| | | | | | | | L: (34/91) | | | |
| | | | | | | | 1.55/4.79E−02 | | | |
| | | | | | | | M-MDD | | | |
| | | | | | | | C: (42/72) | | | |
| | | | | | | | 1.69/2.47E−03 | | | |
| | | | | | | | L: (25/43) | | | |
| | | | | | | | 1.85/3.66E−02 | | | |
| HLA-DQB1 | 212998_x_at | (I) | 8 | NS | Gender/Dx | | Gender/Dx | Alcohol | Antipsychotics | 20 |
| Major | | DE/4 | | | M-SZ | | M-BP | Depression | | |
| Histocompatibility | | 51% | | | C: (11/64) | | L: (34/91) | Longevity | | |
| Complex, Class II, DQ | | | | | 0.68/3.41E−02 | | 1.63/1.30E−02 | Stress | | |
| Beta 1 | | | | | F-MDD | | | Suicide | | |
| | | | | | C: (2/18) | | | SZ | | |
| | | | | | 1/1.23E−02 | | | | | |
| | | | | | M-MDD | | | | | |
| | | | | | L: (13/43) | | | | | |
| | | | | | 0.67/4.28E−02 | | | | | |
| HLA-DQB1 | 211656_x_at | (I) | 8 | NS | Gender/Dx | Gender/Dx | | Alcohol | Antipsychotics | 20 |
| Major | | DE/4 | | | F-MDD | M-MDD | | BP | | |
| Histocompatibility | | 59% | | | C: (2/18) | C: (26/67) | | Depression | | |
| Complex, Class II, DQ | | | | | 1/1.23E−02 | 0.62/4.85E−02 | | Longevity | | |
| Beta 1 | | | | | M-SZ | | | PTSD | | |
| | | | | | C: (11/64) | | | Stress | | |
| | | | | | 0.68/3.15E−02 | | | Suicide | | |
| | | | | | M-SZ | | | SZ | | |
| | | | | | C: (11/64) | | | | | |
| | | | | | 0.74/5.90E−03 | | | | | |
| | | | | | L: (7/39) | | | | | |
| | | | | | 0.72/3.36E−02 | | | | | |
| | | | | | M-MDD | | | | | |
| | | | | | L: (13/43) | | | | | |
| | | | | | 0.69/2.68E−02 | | | | | |
| | | | | | M-PSYCHOSIS | | | | | |
| | | | | | L: (10/56) | | | | | |
| | | | | | 0.69/3.29E−02 | | | | | |
| PNOC | 205901_at | (I) | 4 | NS | Gender/Dx | Gender/Dx | Gender/Dx | Addictions | | 20 |
| Prepronociceptin | | DE/4 | | | M-SZ | M-BP | M-BP | BP | | |
| | | 62% | | | L: (7/39) | L: (9/80) | C: (53/134) | MDD | | |
| | | | | | 0.72/3.36E−02 | 0.68/4.20E−02 | 1.23/4.73E−02 | SZ | | |
| | | | | | | | L: (34/91) | Stress | | |
| | | | | | | | 1.26/2.67E−02 | | | |
| | | | | | | | M-MDD | | | |
| | | | | | | | C: (42/72) | | | |
| | | | | | | | 1.4/2.09E−02 | | | |
| TCF15 | 207306_at | (D) | 2 | NS | Gender/Dx | | All | Suicide | | 20 |
| Transcription Factor | | DE/6 | | | F-MDD | | C: (239/501) | | | |
| 15 (Basic Helix-Loop- | | 94% | | | C: (2/18) | | 1.11/4.85E−02 | | | |
| Helix) | | | | | 0.94/2.46E−02 | | Gender | | | |
| | | | | | M-MDD | | Males | | | |
| | | | | | L: (13/43) | | C: (226/454) | | | |
| | | | | | 0.68/3.21E−02 | | 1.14/2.39E−02 | | | |
| | | | | | | | Gender/Dx | | | |
| | | | | | | | M-BP | | | |
| | | | | | | | L: (34/91) | | | |
| | | | | | | | 2.22/2.61E−03 | | | |
| TOP3A | 214300_s_at | (D) | 4 | NS | Gender/Dx | | All | | Omega-3 | 20 |
| Topoisomerase (DNA) | | DE/4 | | | F-BP | | L: (145/309) | | fatty acids | |
| III Alpha | | 51% | | | C: (4/21) | | 1.18/4.66E−02 | | | |
| | | | | | 0.84/1.97E−02 | | Gender | | | |
| | | | | | | | Males | | | |
| | | | | | | | L: (138/282) | | | |
| | | | | | | | 1.2/3.88E−02 | | | |
| | | | | | | | Gender/Dx | | | |
| | | | | | | | M-SZ | | | |
| | | | | | | | L: (25/64) | | | |
| | | | | | | | 1.75/4.72E−02 | | | |
| (H05785) | 236913_at | (D) | 0 | NS | Gender/Dx | All | | Alcohol | Clozapine | 18 |
| LRRC75A | | AP/6 | | | F-MDD | C: (102/470) | | BP | | |
| Leucine Rich Repeat | | 97% | | | C: (2/18) | 0.56/2.27E−02 | | Suicide | | |
| Containing 75A | | | | | 0.94/2.46E−02 | L: (58/287) | | SZ | | |
| | | | | | | 0.58/3.38E−02 | | | | |
| | | | | | | Gender | | | | |
| | | | | | | Males | | | | |
| | | | | | | C: (95/426) | | | | |
| | | | | | | 0.57/1.64E−02 | | | | |
| | | | | | | L: (54/261) | | | | |
| | | | | | | 0.59/2.71E−02 | | | | |
| | | | | | | Gender/Dx | | | | |
| | | | | | | F-PTSD | | | | |
| | | | | | | C: (2/8) | | | | |
| | | | | | | 1/2.28E−02 | | | | |
| | | | | | | M-PSYCHOSIS | | | | |
| | | | | | | C: (33/198) | | | | |
| | | | | | | 0.65/3.29E−03 | | | | |
| | | | | | | M-SZA | | | | |
| | | | | | | C: (23/97) | | | | |
| | | | | | | 0.68/5.21E−03 | | | | |
| | | | | | | M-SZA | | | | |
| | | | | | | L: (13/55) | | | | |
| | | | | | | 0.66/4.42E−02 | | | | |
| | | | | | | M-MDD | | | | |
| | | | | | | L: (16/39) | | | | |
| | | | | | | 0.76/3.64E−03 | | | | |
| CLSPN | 242150_at | (I) | 0 | NS | Gender/Dx | All | | Suicide | | 18 |
| Claspin | | AP/6 | | | M-PSYCHOSIS | L: (58/287) | | | | |
| | | 95% | | | C: (19/96) | 0.57/4.62E−02 | | | | |
| | | | | | 0.65/2.48E−02 | Gender/Dx | | | | |
| | | | | | | F-MDD | | | | |
| | | | | | | L: (2/6) | | | | |
| | | | | | | 1/3.20E−02 | | | | |
| | | | | | | M-MDD | | | | |
| | | | | | | L: (16/39) | | | | |
| | | | | | | 0.67/4.08E−02 | | | | |
| COL2A1 | 217404_s_at | (D) | 4 | NS | | Gender | Gender/Dx | Aging | | 18 |
| Collagen Type II | | DE/4 | | | | Males | M-PTSD | | | |
| Alpha 1 Chain | | 54% | | | | C: (95/426) | C: (26/31) | | | |
| | | | | | | 0.56/3.53E−02 | 1.83/4.38E−03 | | | |
| | | | | | | Gender/Dx | L: (18/20) | | | |
| | | | | | | M-PSYCHOSIS | 2.3/1.08E−02 | | | |
| | | | | | | C: (33/198) | | | | |
| | | | | | | 0.63/7.32E−03 | | | | |
| | | | | | | M-SZA | | | | |
| | | | | | | C: (23/97) | | | | |
| | | | | | | 0.66/1.08E−02 | | | | |
| | | | | | | L: (13/55) | | | | |
| | | | | | | 0.66/3.73E−02 | | | | |
| HLA-DQB1 | 210747_at | (D) | 8 | NS | | | All | Addiction | Benzodiazepines | 18 |
| Major | | DE/2 | | | | | C: (239/501) | Stress | | |
| Histocompatibility | | 44% | | | | | 1.17/1.03E−02 | | | |
| Complex, Class II, DQ | | | | | | | Gender | | | |
| Beta 1 | | | | | | | Males | | | |
| | | | | | | | C: (226/454) | | | |
| | | | | | | | 1.19/6.06E−03 | | | |
| | | | | | | | Gender/Dx | | | |
| | | | | | | | M-MDD | | | |
| | | | | | | | C: (42/72) | | | |
| | | | | | | | 1.35/3.68E−02 | | | |
| | | | | | | | M-PSYCHOSIS | | | |
| | | | | | | | C: (95/201) | | | |
| | | | | | | | 1.26/1.33E−02 | | | |
| | | | | | | | M-SZA | | | |
| | | | | | | | C: (53/98) | | | |
| | | | | | | | 1.33/2.06E−02 | | | |
| Hs.554262 | 210703_at | (I) | 0 | NS | | All | Gender/Dx | Suicide | | 18 |
| | | AP/6 | | | | C: (102/470) | F-MDD | | | |
| | | 100% | | | | 0.56/2.38E−02 | C: (4/12) | | | |
| | | | | | | L: (58/287) | 7/4.47E−02 | | | |
| | | | | | | 0.58/2.49E−02 | M-MDD | | | |
| | | | | | | Gender | L: (25/43) | | | |
| | | | | | | Males | 2.13/7.30E−03 | | | |
| | | | | | | C: (95/426) | | | | |
| | | | | | | 0.56/4.18E−02 | | | | |
| | | | | | | L: (54/261) | | | | |
| | | | | | | 0.59/1.65E−02 | | | | |
| | | | | | | Gender/Dx | | | | |
| | | | | | | F-MDD | | | | |
| | | | | | | C: (4/11) | | | | |
| | | | | | | 0.82/4.45E−02 | | | | |
| | | | | | | M-BP | | | | |
| | | | | | | C: (18/120) | | | | |
| | | | | | | 0.67/1.08E−02 | | | | |
| | | | | | | M-MDD | | | | |
| | | | | | | L: (16/39) | | | | |
| | | | | | | 0.67/4.08E−02 | | | | |
| PIK3CD | 211230_s_at | (D) | 0 | 1.59E−02/4 | | | All | Alcohol | Clozapine | 18 |
| Phosphatidylinositol- | | DE/6 | | Nominal | | | C: (239/501) | Chronic | Lithium | |
| 4,5-Bisphosphate 3- | | 83% | | | | | 1.13/3.18E−02 | Stress | Valproate | |
| Kinase Catalytic | | | | | | | Gender | Longevity | | |
| Subunit Delta | | | | | | | Males | Suicide | | |
| | | | | | | | C: (226/454) | SZ | | |
| | | | | | | | 1.14/2.71E−02 | | | |
| | | | | | | | Gender/Dx | | | |
| | | | | | | | M-BP | | | |
| | | | | | | | C: (53/134) | | | |
| | | | | | | | 1.3/2.85E−02 | | | |
| | | | | | | | L: (34/91) | | | |
| | | | | | | | 1.57/2.01E−02 | | | |
| | | | | | | | M-MDD | | | |
| | | | | | | | C: (42/72) | | | |
| | | | | | | | 1.65/5.12E−03 | | | |
| SVEP1 | 236927_at | (I) | 4 | 2.17E−02/4 | Gender/Dx | Gender/Dx | | Addiction | Omega-3 | 18 |
| Sushi, Von Willebrand | | DE/2 | | Nominal | F-PTSD | F-MDD | | SZ | fatty acids | |
| Factor Type A, EGF | | 49% | | | C: (5/12) | C: (4/11) | | | | |
| And Pentraxin | | | | | 0.8/4.41E−02 | 0.82/4.41E−02 | | | | |
| Domain Containing 1 | | | | | M-PTSD | | | | | |
| | | | | | C: (13/38) | | | | | |
| | | | | | 0.67/4.68E−02 | | | | | |
| TNFRSF11B | 204932_at | (D) | 4 | 2.67E−02/4 | Gender/Dx | | Gender/Dx | Stress | | 18 |
| TNF Receptor | | DE/2 | | Nominal | F-BP | | M-MDD | PTSD | | |
| Superfamily Member | | 37% | | | C: (4/21) | | C: (42/72) | | | |
| 11b | | | | | 0.81/3.00E−02 | | 1.42/4.25E−02 | | | |
| | | | | | M-MDD | | L: (25/43) | | | |
| | | | | | L: (13/43) | | 1.59/3.84E−02 | | | |
| | | | | | 0.71/1.72E−02 | | | | | |
| ZNF91 | 244259_s_at | (I) | 0 | 6.37E−01/2 | | Gender/Dx | Gender | Alcohol | | 18 |
| Zinc Finger Protein 91 | | AP/6 | | Stepwise | | F-MDD | Females | Circadian | | |
| | | 95% | | | | C: (4/11) | C: (13/47) | abnormalities | | |
| | | | | | | 0.93/1.17E−02 | 2.12/1.03E−02 | PTSD | | |
| | | | | | | | Gender/Dx | | | |
| | | | | | | | F-BP | | | |
| | | | | | | | C: (2/16) | | | |
| | | | | | | | 4.21/4.55E−02 | | | |
| | | | | | | | M-BP | | | |
| | | | | | | | C: (53/134) | | | |
| | | | | | | | 1.35/1.26E−02 | | | |
| CDK6 | 224851_at | (I) | 4 | NS | Gender/Dx | All | | Alcohol | | 17 |
| Cyclin Dependent | | DE/4 | | | F-BP | C: (102/470) | | ASD | | |
| Kinase 6 | | 56% | | | C: (4/21) | 0.57/1.03E−02 | | Circadian | | |
| | | (I) | | | 0.78/4.44E−02 | Gender | | abnormalities | | |
| | | AP/2 | | | L: (3/11) | Males | | Longevity | | |
| | | 42% | | | 1/7.15E−03 | C: (95/426) | | MDD | | |
| | | | | | | 0.59/5.57E−03 | | SZ | | |
| | | | | | | Gender/Dx | | | | |
| | | | | | | M-MDD | | | | |
| | | | | | | C: (26/67) | | | | |
| | | | | | | 0.67/9.11E−03 | | | | |
| EDN1 | 1564630_at | (I) | 4 | 8.69E−02/2 | | | Gender | | | 16 |
| Endothelin 1 | | AP/4 | | Stepwise | | | Females | | | |
| | | 56% | | | | | C: (13/47) | | | |
| | | | | | | | 1.9/1.48E−02 | | | |
| | | | | | | | Gender/Dx | | | |
| | | | | | | | M-BP | | | |
| | | | | | | | C: (53/134) | | | |
| | | | | | | | 1.27/2.37E−02 | | | |
| (AF090920) | 234739_at | (I) | 0 | NS | Gender | | Gender/Dx | | | 16 |
| PPFIBP2 | | AP/6 | | | Female | | M-PSYCHOSIS | | | |
| PPFIA Binding Protein | | 94% | | | C: (16/65) | | C: (95/201) | | | |
| 2 | | | | | 0.68/1.42E−02 | | 1.19/3.77E−02 | | | |
| | | | | | L: (10/36) | | M-SZ | | | |
| | | | | | 0.69/3.87E−02 | | C: (42/103) | | | |
| | | | | | Gender/Dx | | 1.22/4.66E−02 | | | |
| | | | | | F-PTSD | | | | | |
| | | | | | C: (5/12) | | | | | |
| | | | | | 0.8/4.41E−02 | | | | | |
| DCAF12 | 224789_at | (D) | 2 | NS | Gender/Dx | | Gender/Dx | Cocaine | Omega-3 | 16 |
| DDB1 And CUL4 | | DE/6 | | | F-MDD | | M-BP | Suicide | fatty acids | |
| Associated Factor 12 | | 86% | | | C: (2/18) | | C: (53/134) | | Clozapine | |
| | | | | | 1/1.23E−02 | | 1.61/4.42E−03 | | | |
| DNAJC18 | 227166_at | (I) | 0 | NS | Gender | Gender/Dx | | BP | | 16 |
| DnaJ Heat Shock | | DE/6 | | | Female | F-MDD | | | | |
| Protein Family | | 94% | | | L: (10/36) | C: (4/11) | | | | |
| (Hsp40) Member C18 | | | | | 0.78/4.97E−03 | 0.93/1.17E−02 | | | | |
| | | | | | Gender/Dx | | | | | |
| | | | | | F-SZA | | | | | |
| | | | | | L: (3/8) | | | | | |
| | | | | | 0.93/2.63E−02 | | | | | |
| | | | | | F-BP | | | | | |
| | | | | | L: (3/11) | | | | | |
| | | | | | 0.88/3.31E−02 | | | | | |
| | | | | | F-PSYCHOSIS | | | | | |
| | | | | | L: (3/8) | | | | | |
| | | | | | 0.93/2.63E−02 | | | | | |
| | | | | | F-PTSD | | | | | |
| | | | | | L: (3/6) | | | | | |
| | | | | | 1/2.48E−02 | | | | | |
| HLA-DRB1 | 208306_x_at | (I) | 4 | NS | Gender/Dx | Gender/Dx | | Stress | Antipsychotics | 16 |
| Major | | AP/4 | | | F-MDD | M-SZA | | PTSD | | |
| Histocompatibility | | 52% | | | C: (2/18) | C: (23/97) | | | | |
| Complex, Class II, DR | | | | | 0.91/3.39E−02 | 0.62/4.69E−02 | | | | |
| Beta 1 | | | | | M-MDD | | | | | |
| | | | | | L: (13/43) | | | | | |
| | | | | | 0.66/4.79E−02 | | | | | |
| | | | | | M-SZ | | | | | |
| | | | | | L: (7/39) | | | | | |
| | | | | | 0.71/4.27E−02 | | | | | |
| SEPT7P2 | 1569973_at | (I) | 0 | NS | Gender | | Gender/Dx | Suicide | | 16 |
| Septin 7 Pseudogene | | DE/6 | | | Females | | M-MDD | | | |
| 2 | | 100% | | | C: (16/65) | | C: (42/72) | | | |
| | | (I) | | | 0.65/3.27E−02 | | 1.45/1.37E−02 | | | |
| | | AP/2 | | | Gender/Dx | | L: (25/43) | | | |
| | | 39% | | | F-PTSD | | 2.25/5.24E−04** | | | |
| | | | | | C: (5/12) | | M-PTSD | | | |
| | | | | | 0.97/3.69E−03 | | C: (26/31) | | | |
| | | | | | M-SZ | | 2.38/7.38E−04** | | | |
| | | | | | C: (11/64) | | L: (18/20) | | | |
| | | | | | 0.77/2.83E−03 | | 3.59/1.77E−03 | | | |
| VEGFA | 212171_x_at | (I) | 4 | NS | Gender/Dx | | Gender/Dx | BP | Lithium | 16 |
| Vascular Endothelial | | AP/4 | | | M-PSYCHOSIS | | M-MDD | MDD | Valproate | |
| Growth Factor A | | 65% | | | C: (19/96) | | C: (42/72) | Stress | Olanzapine | |
| | | | | | 0.66/1.78E−02 | | 1.33/4.83E−02 | SZ | | |
| | | | | | M-SZA | | | | | |
| | | | | | C: (8/32) | | | | | |
| | | | | | 0.7/4.48E−02 | | | | | |
| WNK1 | 1555068_at | (D) | 2 | NS | Gender/Dx | | Gender/Dx | Alcohol | Omega-3 | 16 |
| WNK Lysine Deficient | | DE/6 | | | M-MDD | | M-BP | Depression | Fatty acids | |
| Protein Kinase 1 | | 92% | | | L: (13/43) | | C: (53/134) | Suicide | SSRI | |
| | | | | | 0.77/2.75E−03 | | 1.41/3.18E−02 | Methamphetamine | | |
| | | | | | | | | Stress | | |
| (AF087971) | 1561067_at | (I) | 0 | NS | | All | | BP | | 14 |
| PBRM1 | | AP/6 | | | | C: (102/470) | | Hallucinations | | |
| Polybromo 1 | | 90% | | | | 0.56/3.71E−02 | | Longevity | | |
| | | | | | | Gender | | MDD | | |
| | | | | | | Males | | Methamphetamine | | |
| | | | | | | C: (95/426) | | Mood | | |
| | | | | | | 0.56/2.87E−02 | | Psychosis | | |
| | | | | | | Gender/Dx | | Stress | | |
| | | | | | | M-BP | | Suicide | | |
| | | | | | | C: (18/120) | | | | |
| | | | | | | 0.63/3.95E−02 | | | | |
| | | | | | | M-PSYCHOSIS | | | | |
| | | | | | | C: (33/198) | | | | |
| | | | | | | 0.63/8.63E−03 | | | | |
| | | | | | | M-SZA | | | | |
| | | | | | | C: (23/97) | | | | |
| | | | | | | 0.66/1.26E−02 | | | | |
| (Hs.609761) | 244331_at | (D) | 0 | NS | Gender/Dx | | Gender/Dx | Alcohol | Omega-3 | 14 |
| SFPQ | | DE/6 | | | M-SZ | | M-MDD | BP | fatty acids | |
| Splicing Factor Proline | | 98% | | | C: (11/64) | | C: (42/72) | MDD | Clozapine | |
| And Glutamine Rich | | | | | 0.68/3.28E−02 | | 1.68/7.35E−03 | Stress | Antidepressants | |
| | | | | | L: (7/39) | | | Suicide | Antipsychotics | |
| | | | | | 0.75/2.21E−02 | | | | | |
| (Hs.659426) | 240599_x_at | (D) | 0 | NS | Gender/Dx | | Gender/Dx | Suicide | | 14 |
| PHC3 | | DE/6 | | | F-MDD | | M-MDD | | | |
| Polyhomeotic | | 92% | | | C: (2/18) | | C: (42/72) | | | |
| Homolog 3 | | | | | 0.91/3.39E−02 | | 1.48/1.83E−02 | | | |
| CCDC85C | 219018_s_at | (D) | 2 | NS | Gender | | | Suicide | | 14 |
| Coiled-Coil Domain | | DE/6 | | | Female | | | | | |
| Containing 85C | | 94% | | | L: (10/36) | | | | | |
| | | | | | 0.7/3.31E−02 | | | | | |
| | | | | | Gender/Dx | | | | | |
| | | | | | F-BP | | | | | |
| | | | | | C: (4/21) | | | | | |
| | | | | | 0.79/3.66E−02 | | | | | |
| | | | | | L: (3/11) | | | | | |
| | | | | | 0.92/2.06E−02 | | | | | |
| | | | | | F-PTSD | | | | | |
| | | | | | L: (3/6) | | | | | |
| | | | | | 1/2.48E−02 | | | | | |
| GSPT1 | 215438_x_at | (D) | 0 | NS | Gender/Dx | | Gender/Dx | BP | Valproate | 14 |
| G1 To S Phase | | DE/6 | | | F-MDD | | M-BP | Suicide | | |
| Transition 1 | | 94% | | | C: (2/18) | | C: (53/134) | MDD | | |
| | | | | | 1/1.23E−02 | | 1.58/4.92E−03 | | | |
| HLA-DQB1 | 211654_x_at | (I) | 8 | NS | Gender/Dx | | | Alcohol | Antipsychotics | 14 |
| Major | | DE/2 | | | M-PSYCHOSIS | | | BP | | |
| Histocompatibility | | 40% | | | L: (10/56) | | | Depression | | |
| Complex, Class II, DQ | | | | | 0.73/1.23E−02 | | | Longevity | | |
| Beta 1 | | | | | M-SZ | | | PTSD | | |
| | | | | | L: (7/39) | | | Stress | | |
| | | | | | 0.81/5.78E−03 | | | Suicide | | |
| | | | | | | | | SZ | | |
| LOXL2 | 228808_s_at | (D) | 4 | NS | Gender | | | BP | | 14 |
| Lysyl Oxidase Like 2 | | DE/4 | | | Females | | | Suicide | | |
| | | 59% | | | C: (16/65) | | | | | |
| | | | | | 0.66/3.05E−02 | | | | | |
| | | | | | Gender/Dx | | | | | |
| | | | | | F-MDD | | | | | |
| | | | | | C: (2/18) | | | | | |
| | | | | | 1/1.23E−02 | | | | | |
| MBNL3 | 219814_at | (D) | 0 | NS | Gender/Dx | | Gender/Dx | Psychosis | | 14 |
| Muscleblind Like | | DE/6 | | | M-MDD | | M-BP | Hallucination | | |
| Splicing Regulator 3 | | 92% | | | L: (13/43) | | C: (53/134) | | | |
| | | | | | 0.71/1.51E−02 | | 1.43/8.16E−03 | | | |
| PTN | 211737_x_at | (D) | 0 | NS | | | All | SZ | Omega-3 | 14 |
| Pleiotrophin | | DE/6 | | | | | C: (239/501) | Stress | fatty acids | |
| | | 92% | | | | | 1.16/1.17E−02 | Suicide | Risperidone | |
| | | | | | | | Gender | | | |
| | | | | | | | Males | | | |
| | | | | | | | C: (226/454) | | | |
| | | | | | | | 1.2/4.66E−03 | | | |
| | | | | | | | Gender/Dx | | | |
| | | | | | | | M-PSYCHOSIS | | | |
| | | | | | | | C: (95/201) | | | |
| | | | | | | | 1.24/1.98E−02 | | | |
| | | | | | | | M-SZA | | | |
| | | | | | | | C: (53/98) | | | |
| | | | | | | | 1.35/1.28E−02 | | | |
| RALGAPA2 | 231826_at | (D) | 0 | NS | Gender/Dx | | Gender/Dx | BP | | 14 |
| Ral GTPase Activating | | DE/6 | | | F-MDD | | M-MDD | | | |
| Protein Catalytic | | 97% | | | C: (2/18) | | C: (42/72) | | | |
| Alpha Subunit 2 | | | | | 0.94/2.46E−02 | | 2.06/4.52E−04** | | | |
| | | | | | | | L: (25/43) | | | |
| | | | | | | | 2.05/5.35E−03 | | | |
| YBX3 | 201160_s_at | (D) | 0 | NS | Gender/Dx | | Gender/Dx | BP | Mianserin | 14 |
| Y-Box Binding Protein | | DE/6 | | | F-MDD | | M-BP | Suicide | | |
| 3 | | 94% | | | C: (2/18) | | C: (53/134) | SZ | | |
| | | | | | 0.97/1.75E−02 | | 1.39/1.23E−02 | | | |
| ZNF441 | 1553193_at | (I) | 0 | NS | | Gender/Dx | Gender/Dx | | | 14 |
| Zinc Finger Protein | | AP/6 | | | | M-SZA | M-MDD | | | |
| 441 | | 95% | | | | L: (13/55) | L: (25/43) | | | |
| | | (I) | | | | 0.67/3.13E−02 | 1.72/1.92E−02 | | | |
| | | DE/2 | | | | | | | | |
| | | 35% | | | | | | | | |
| CCND1 | 208712_at | (D) | 4 | NS | | | Gender/Dx | Addiction | | 12 |
| Cyclin D1 | | DE/4 | | | | | M-BP | MDD | | |
| | | 57% | | | | | C: (53/134) | Stress | | |
| | | | | | | | 1.33/4.53E−02 | Hallucinogens | | |
| CDK6 | 224847_at | (I) | 4 | NS | | | Gender/Dx | Alcohol | | 12 |
| Cyclin Dependent | | DE/4 | | | | | M-PTSD | ASD | | |
| Kinase 6 | | 63% | | | | | L: (18/20) | Circadian | | |
| | | | | | | | 2.09/1.75E−02 | abnormalities | | |
| | | | | | | | | Longevity | | |
| | | | | | | | | MDD | | |
| | | | | | | | | SZ | | |
| COMT | 213981_at | (D) | 4 | NS | Gender/Dx | | | ADHD | Clozapine | 12 |
| Catechol-O- | | DE/4 | | | M-MDD | | | Aggression | Morphine | |
| Methyltransferase | | 54% | | | L: (13/43) | | | Alcohol | Mood | |
| | | | | | 0.71/1.41E−02 | | | Anxiety | Stabilizers | |
| | | | | | | | | BP | | |
| | | | | | | | | Chronic | | |
| | | | | | | | | Stress | | |
| | | | | | | | | MDD | | |
| | | | | | | | | OCD | | |
| | | | | | | | | Panic | | |
| | | | | | | | | Disorder | | |
| | | | | | | | | Psychosis | | |
| | | | | | | | | PTSD | | |
| | | | | | | | | Suicide | | |
| | | | | | | | | SZ | | |
| HTR2A | 211616_s_at | (D) | 4 | NS | Gender/Dx | | | Addictions | | 12 |
| 5-Hydroxytryptamine | | DE/4 | | | M-BP | | | Aging | | |
| Receptor 2A | | 52% | | | L: (16/81) | | | Alcohol | | |
| | | | | | 0.65/2.89E−02 | | | Anxiety | | |
| | | | | | | | | BP | | |
| | | | | | | | | Depression | | |
| | | | | | | | | MDD | | |
| | | | | | | | | Mood | | |
| | | | | | | | | Disorders | | |
| | | | | | | | | NOS | | |
| | | | | | | | | OCD | | |
| | | | | | | | | Panic | | |
| | | | | | | | | Disorder | | |
| | | | | | | | | PTSD | | |
| | | | | | | | | Stress | | |
| | | | | | | | | Suicide | | |
| | | | | | | | | SZ | | |
| NF1 | 212676_at | (I) | 4 | NS | Gender/Dx | | | Addiction | Fluoxetine | 12 |
| Neurofibromin 1 | | DE/4 | | | F-BP | | | BP | SSRI | |
| | | 59% | | | L: (3/11) | | | PTSD | | |
| | | | | | 0.92/2.06E−02 | | | | | |
| SHMT1 | 217304_at | (D) | 6 | NS | | Gender/Dx | | Suicide | Clozapine | 12 |
| Serine | | DE/2 | | | | F-PTSD | | | | |
| Hydroxymethyltransferase | | 43% | | | | C: (2/8) | | | | |
| 1 | | | | | | 1/2.28E−02 | | | | |
| | | | | | | M-SZA | | | | |
| | | | | | | L: (13/55) | | | | |
| | | | | | | 0.7/1.54E−02 | | | | |
| TSPO | 202096_s_at | (I) | 6 | NS | Gender/Dx | | | SZ | | 12 |
| Translocator Protein | | DE/2 | | | M-SZ | | | | | |
| | | 38% | | | C: (11/64) | | | | | |
| | | | | | 0.72/1.06E−02 | | | | | |
| DENND1B | 1557309_at | (I) | 0 | NS | Gender/Dx | | | | Omega-3 | 10 |
| DENN Domain | | DE/6 | | | M-SZA | | | | | |
| Containing 1B | | 90%; (I) | | | L: (3/17) | | | | | |
| | | AP/2 | | | 0.83/3.89E−02 | | | | | |
| | | 40% | | | | | | | | |
| MCRS1 | 202556_s_at | (I) | 0 | NS | Gender/Dx | | | MDD | | 10 |
| Microspherule Protein | | DE/6 | | | M-MDD | | | | | |
| 1 | | 90% | | | L: (13/43) | | | | | |
| | | | | | 0.75/5.16E−03 | | | | | |
| OSBP2 | 1569617_at | (D) | 0 | NS | Gender/Dx | | | Cocaine | | 10 |
| Oxysterol Binding | | DE/6 | | | F-MDD | | | Suicide | | |
| Protein 2 | | 94% | | | C: (2/18) | | | SZ | | |
| | | | | | 1/1.23E−02 | | | | | |
| FAM134B | 218510_x_at | (I) | 4 | NS | | | | Antisocial | Omega-3 | 8 |
| Family With Sequence | | DE/4 | | | | | | Personality | Fatty acids | |
| Similarity 134 | | 51%; (I) | | | | | | Suicide | | |
| Member B | | AP/2 | | | | | | | | |
| | | 34% | | | | | | | | |
| ZNF429 | 1561270_at | (D) | 6 | NS | | | | | | 8 |
| Zinc Finger Protein | | DE/2 | | | | | | | | |
| 429 | | 37% | | | | | | | | |
| (Hs.677263) | 216444_at | (D) | 0 | NS | | | | Aging | | 6 |
| SMURF2 | | AP/6 | | | | | | Suicide | | |
| SMAD Specific E3 | | 100% | | | | | | Stress | | |
| Ubiquitin Protein | | (D) | | | | | | | | |
| Ligase 2 | | DE/4 | | | | | | | | |
| | | 71% | | | | | | | | |

DE—differential expression, AP—Absent/Present. NS—Non-stepwise in validation. For Predictions, C—cross-sectional (using levels from one visit), L—longitudinal (using levels and slopes from multiple visits). In All, by Gender, and personalized by Gender and Diagnosis (Gender/Dx). M—males, F—Females. MDD—depression, BP— bipolar, SZ—schizophrenia, SZA—schizoaffective, PSYCHOSIS—schizophrenia and schizoaffective combined, PTSD—post-traumatic stress disorder. Bold and **—significant after Bonferroni correction for the number of biomarkers tested (65). For Steps 2, 5 and 6, see Supplementary Information tables for citations for the evidence.

TABLE 2
Therapeutics. New Drug Discovery/Repurposing.
A. CMAP Top Biomarkers (n = 65 probesets; 19 decreased, 14 increased are present in HG-U133A array used by CMAP)

| rank | CMAP name | score | Description |
|---|---|---|---|
| 1 | SC-560 | −1 | SC-560 is an NSAID, member of the diaryl heterocycle class of |
| | | | cyclooxygenase (COX) inhibitors which includes celecoxib (Celebrex ™) |
| | | | and rofecoxib (Vioxx ™). However, unlike these selective COX-2 |
| | | | inhibitors, SC-560 is a selective inhibitor of COX-1. |
| 2 | pyridoxine | −0.997 | Pyridoxine is the 4-methanol form of vitamin B6 and is converted to |
| | | | pyridoxal 5-phosphate in the body. Pyridoxal 5-phosphate is a coenzyme |
| | | | for synthesis of amino acids, neurotransmitters (serotonin, |
| | | | norepinephrine), sphingolipids, aminolevulinic acid. |
| 3 | methylergometrine | −0.975 | Methylergometrine is a synthetic analogue of ergonovine, a psychedelic |
| | | | alkaloid found in ergot, and many species of morning glory. It is |
| | | | chemically similar to LSD, ergine, ergometrine, and lysergic acid. Due to |
| | | | its oxytocic properties, it has a medical use in obstetrics. |
| 4 | LY-294002 | −0.923 | LY-294002 is a potent, cell permeable inhibitor of phosphatidylinositol |
| | | | 3-kinase (PI3K) that acts on the ATP binding site of the enzyme. The |
| | | | PI3K pathway has a role in inhibiting apoptosis in cancer. PI3K is also |
| | | | known to regulate TLR-mediated inflammatory responses. |
| 5 | haloperidol | −0.917 | Widely used typical anti-psychotic medication |
| 6 | cytisine | −0.909 | Like varenicline, cytisine is a partial agonist of nicotinic acetylcholine |
| | | | receptors (nAChRs), with an affinity for the α4β2 receptor subtype, and a |
| | | | half-life of 4.8 hours. |
| 7 | cyanocobalamin | −0.902 | Cyanocobalamin is a form of vitamin B12. Vitamin B12 is important for |
| | | | growth, cell reproduction, blood formation, and protein and tissue |
| | | | synthesis. |
| 8 | apigenin | −0.899 | Apigenin (4′,5,7-trihydroxyflavone), found in many plants such as |
| | | | chamomile, is a natural product belonging to the flavone class. Apigenin |
| | | | acts as a monoamine transporter activator, and is a weak ligand for |
| | | | central benzodiazepine receptors in vitro and exerts anxiolytic and slight |
| | | | sedative effects in an animal model. It has also effects on adenosine |
| | | | receptors and is an acute antagonist at the NMDA receptors (IC50 = 10 |
| | | | μM). In addition, like various other flavonoids, apigenin has been found |
| | | | to possess nanomolar affinity for the opioid receptors, acting as a non- |
| | | | selective antagonist of all three opioid receptors. |
| 9 | [symbol] | −0.892 | Escin, a natural mixture of triterpenoid saponins isolated from horse |
| | | | chestnut (Aesculus hippocastanum) seeds, is used and studied as a |
| | | | vasoprotective anti-inflammatory, anti-edematous and anti-nociceptive |
| | | | agent. |
| 13 | amoxapine | −0.875 | Amoxapine is a tricyclic antidepressant of the dibenzoxazepine class. |
| | | | This drug is used to treat symptoms of depression and neuropathic pain. |

B. L1000CDS2 Top Biomarkers (n = 60 unique genes; 26 increased and 34 decreased).

| Rank | Score | Drug | Description |
|---|---|---|---|
| 1 | 0.1458 | Quinethazone | Thiazide diuretic |
| 2 | 0.1458 | [symbol] | Related to the green tea compound EGCG and |
| | | | possible therapeutic molecule for NP treatment due |
| | | | to its anti-inflammatory and antioxidant properties. |
| | | | Interestingly, it has been shown that EGCG reduced |
| | | | bone cancer pain. |
| 3 | 0.125 | [symbol] | Omega-3 fatty acid |
| 4 | 0.125 | LFM-A13 | Tyrosine kinase inhibitor with anti-inflamatory |
| | | | properties |
| 5 | 0.125 | Picrotoxinin | GABA and glycine receptors inhibitor |
| 6 | 0.125 | INDAPAMIDE | Thiazide-like diuretic |
| 7 | 0.125 | BRD-K15318909 | |
| 8 | 0.125 | BRD-K53011428 | |
| 9 | 0.125 | BRD-K35100517 | |
| 10 | 0.125 | MLS-0454435.0001 | |
| 11 | 0.125 | NCGC00181213-02 | |
| 12 | 0.125 | ST003833 | |
| 13 | 0.125 | STOCK2S-84516 | |
| 14 | 0.125 | MLS-0390932.0001 | |
| 15 | 0.125 | BRD-K98143437 | |
| 16 | 0.125 | BRD-A00993607 | |
| 17 | 0.125 | BRD-K68103045 | |
| 18 | 0.125 | BRD-K90700939 | |
| 19 | 0.125 | triamterene | potassium-sparing diuretic used in combination with |
| | | | thiazide diuretics for the treatment of hypertension |
| | | | and edema. |
| 20 | 0.1042 | PSEUDOEPHEDRINE HYDROCHLORIDE | sympathomimetic drug |
| 21 | 0.1042 | [symbol] | Omega-3 fatty acid with antihyperalgesic effect in |
| | | | neuropathic pain |
| 22 | 0.1042 | Evoxine | Plant alkaloid with hypnotic and sedative effects. |
| 23 | 0.1042 | Gavestinel | NMDA receptor antagonist |
| 24 | 0.1042 | Mometasone furoate | Corticosteroid |
| 25 | 0.1042 | ZM 241385 | denosine A2A receptor antagonist |

A. Connectivity Map (CMAP) analysis-drugs that have opposite gene expression profile effects to pain biomarkers signatures. Out of 65 probesets, 14 of the 29 increased, and 19 of the 36 decreased were present in HG-U133A array used by Connectivity Map. A score of −1 indicates the perfect opposite match, i.e., the best potential therapeutic for Pain. B. NIH LINCS analysis using the L1000CDS2 (LINCS L1000 Characteristic Direction Signature Search Engine) tool. Query for signature is done using gene symbols and direction of change Shown are compounds mimicking direction of change in high memory. A higher score indicates a better match. Bold-drugs known to treat pain, which thus serve as a de facto positive control for the Example. Italic—natural compounds.

TABLE 3
Demographics.

| | | | | | | |
|---|---|---|---|---|---|---|
| | | | | | Age at time | |
| | Number of | | | | of visit | T-test |
| Cohorts | subjects | Gender | Diagnosis | Ethnicity | Mean (SD) | for age |

Discovery

| | | | | | |
|---|---|---|---|---|---|
| Discovery Cohort | 28 | Male = 19 | BP = 9 | EA = 17 | 52 |
| (Longitudinal Within-Subject | (with 79 | Female = 9 | MDD = 3 | AA = 10 | (7.94) |
| Changes in Pain Scale) | visits) | | SZA = 6 | Mixed = 1 | |
| Low Pain 0-2 to | | | SZ = 3 | | |
| High Pain 6-10 | | | PTSD = 5 | | |
| | | | PSYCH = 2 | | |

Validation

| | | | | | |
|---|---|---|---|---|---|
| Independent Validation Cohort | 23 | Male = 13 | MDD = 8 | EA = 17 | 51.9 |
| (Clinical Severe Pain | (30 visits) | Female = 10 | BP = 6 | AA = 6 | (7.1) |
| Diagnosis | | | SZ = 2 | | |
| SF36 sum of scores on | | | SZA = 2 | | |
| questions 21 and 22 ≥10 | | | PTSD = 2 | | |
| Pain Scale ≥6) | | | MOOD = 3 | | |

Testing

| | | | | | | |
|---|---|---|---|---|---|---|
| Independent Testing Cohort | 162 | Male = 134 | BP = 52 | EA = 112 | 50.3 | High Pain |
| For Predicting State | (411 visits) | Female = 28 | MDD = 39 | AA = 48 | (8.97) | (n = 101) |
| (High Pain State Pain Scale ≥6 | | | SZA = 19 | Hispanic = 2 | Others | Vs. Others |
| at Time of Assessment) | | | SZ = 26 | | 50.12 | (n = 310) |
| | | | PTSD = 20 | | High Pain | 0.824 |
| | | | MOOD = 4 | | 50.50 | |
| | | | PSYCH = 2 | | | |
| Independent Testing Cohort | 181 | Male = 163 | BP = 46 | EA = 117 | 52.45 | ED visits |
| For Predicting Trait | (470 visits) | Female = 18 | MDD = 33 | AA = 62 | (6.13) | for Pain |
| (Future ED visits for Pain in | | | SZA = 45 | Hispanic = 2 | Others | (n = 102) |
| the First Year Following | | | SZ = 38 | | 52.61 | vs. Others |
| Assessment) | | | PTSD = 13 | | ED visits | (n = 368) |
| | | | MOOD = 4 | | for Pain | 0.237 |
| | | | PSYCH = 2 | | 51.87 | |
| Independent Testing Cohort | 189 | Male = 170 | BP = 49 | EA = 124 | 51.79 | ED visits |
| For Predicting Trait | (501 visits) | Female = 19 | MDD = 34 | AA = 62 | (6.75) | for Pain |
| (Future ED visits for Pain in All | | | SZA = 45 | Hispanic = 3 | Others | (n = 239) |
| Years Following Assessment) | | | SZ = 40 | | 51.58 | vs. Others |
| | | | PTSD = 15 | | ED visits | (n = 262) |
| | | | MOOD = 4 | | for Pain | 0.4720 |
| | | | PSYCH = 2 | | 52.02 | |

MDD—depression, BP—bipolar, SZ—schizophrenia, SZA—schizoaffective, PSYCHOSIS—schizophrenia and schizoaffective combined, PTSD—post-traumatic stress disorder.

TABLE 4
Top Biomarkers for Pain

| | | | | | | | | | | |
|---|---|---|---|---|---|---|---|---|---|---|
| | | Discovery | | | | Prior Non- | Prior Non- | Prior Non- | Prioritization | |
| Gene Symbol/ | | (Change) | Prior Human | Prior Human | Prior Human | human | human Nervous | human | Total CFG | Validation |
| Gene Name | | Method/ | Genetic | Nervous Tissue | Peripheral | Genetic | Tissue | Peripheral | Score | Anova p- |
| Name | Probeset | Score | Evidence | Evidence | Evidence | Evidence | Evidence | Evidence | For Pain | value |

| | | | | | | | | | |
|---|---|---|---|---|---|---|---|---|---|
| HLA-DQB1 | 212998_x_at | (I) | | (D) DRG | (D)Blood | (I) Spinal Cord | | 12 | NS |
| Major | | DE/4 | | Neurological | Neurological | Neuropathic | | | |
| Histocompatibility | | 51% | | Pain ¹ | Pain ² | Pain ³ | | | |
| Complex, Class II, | | | | | | | | | |
| DQ Beta 1 | | | | | | | | | |
| HLA-DQB1 | 211656_x_at | (I) | | (D) DRG | (D) Blood | (I) Spinal Cord | | 12 | NS |
| Major | | DE/4 | | Neurological | Neurological | Neuropathic | | | |
| Histocompatibility | | 59% | | Pain ¹ | Pain ² | Pain ³ | | | |
| Complex, Class II, | | | | | | | | | |
| DQ Beta 1 | | | | | | | | | |
| CALCA | 210727_at | (D) | Analgesia⁴ | | (D) Vertebral | (I) DRG | (I) blood | 11 | NS |
| Calcitonin Related | | DE/4 | Migraine ⁵ | | disc, | Pain ⁹ | Acute Pain ¹² | | |
| Polypeptide Alpha | | 54% | | | Neurological | (I) Neurological | | | |
| | | | | | Pain ⁶ | Pain ¹⁰ | | | |
| | | | | | (D) | (I) Dorsal Horn | | | |
| | | | | | Blood | Neurological | | | |
| | | | | | Neuropathic | Pain ¹¹ | | | |
| | | | | | Pain⁷ | | | | |
| | | | | | (I) | | | | |
| | | | | | Migraine/ | | | | |
| | | | | | Headache ⁸ | | | | |
| CCDC144B | 1557366_at | (D) | | (I) | | (D) NAC | | 10 | NS |
| Coiled-Coil Domain | | DE/4 | | Neurological | | Neuropathic | | | |
| Containing 144B | | 56% | | Pain ¹ | | Pain ¹³ | | | |
| (Pseudogene) | | | | | | | | | |
| CNTN1 | 1554784_at | (D) | | (D) DRG | (D) | | | 10 | NS |
| Contactin 1 | | DE/4 | | Neuropathy¹⁴ | CSF¹⁵ | | | | |
| | | 52% | | | | | | | |
| GNG7 | 1566643_a_at | (D) | | (I) sural nerve | (I) vertebral | | | 10 | 6.81E−02 |
| G Protein Subunit | | DE/4 | | Diabetic | disc | | | | Stepwise |
| Gamma 7 | | 59% | | Neuropathy ¹⁶ | Neurological | | | | |
| | | | | | Pain ⁶ | | | | |
| HLA-DQB1 | 210747_at | (D) | | (D) DRG | (D) Whole | (I) Spinal Cord | | 10 | NS |
| Major | | DE/2 | | Neurological | blood | Neuropathic | | | |
| Histocompatibility | | 44% | | Pain ¹ | Neurological | Pain ³ | | | |
| Complex, Class II, | | | | | Pain ² | | | | |
| DQ Beta 1 | | | | | | | | | |
| HLA-DQB1 Major | 211654_x_at | (I) | | (D) DRG | (D) Whole | (I) Spinal Cord | | 10 | NS |
| Histocompatibility | | DE/2 | | Neurological | blood | Neuropathic | | | |
| Complex, Class II, | | 40% | | Pain ¹ | Neurological, | Pain ³ | | | |
| DQ Beta 1 | | | | | Pain ² | | | | |
| ASTN2 | 1554816_at | (I) | Chronic | | | | | 8 | 1.71E−01 |
| Astrotactin 2 | | DE/6 | Migraine ¹⁷, ¹⁸, ¹⁹, ²⁰ | | | | | | Stepwise |
| | | 83% | | | | | | | |
| CASP6 | 209790_s_at | (I) | | | (I) vertebral | DRG | | 8 | NS |
| Caspase 6 | | DE/4 | | | disc | Neuropathic | | | |
| | | 51% | | | Neurological ⁶ | pain ²¹ | | | |
| CCDC85C | 219018_s_at | (D) | | | | (I) | | 8 | NS |
| Coiled-Coil Domain | | DE/6 | | | | PAG | | | |
| Containing 85C | | 94% | | | | Neuropathic | | | |
| | | | | | | Pain ¹³ | | | |
| CCND1 | 208712_at | (D) | | | (D) Serum | (I) (DRG) | | 8 | NS |
| Cyclin D1 | | DE/4 | | | Chronic Pain ²² | Neurological | | | |
| | | 57% | | | | Pain ¹⁰ | | | |
| CDK6 | 224851_at | (I) | | | (D) Serum | (I) | | 8 | NS |
| Cyclin Dependent | | DE/4 | | | Chronic Pain ²² | Neuropathic | | | |
| Kinase 6 | | 56% | | | | Pain ²³ | | | |
| | | (I) | | | | | | | |
| | | AP/2 | | | | | | | |
| | | 42% | | | | | | | |
| CDK6 | 224847_at | (I) | | | (D) Serum | (I) | | 8 | NS |
| Cyclin Dependent | | DE/4 | | | Chronic Pain ²² | Neuropathic | | | |
| Kinase 6 | | 63% | | | | Pain ²³ | | | |
| COL27A1 | 225293_at | (D) | | | (D) | (I) PAG | | 8 | 7.47E−01 |
| Collagen Type | | DE/4 | | | Lymphoblast | Neuropathic | | | Stepwise |
| XXVII Alpha 1 | | 79% | | | Migraine ²⁴ | Pain ¹³ | | | |
| Chain | | | | | | | | | |
| COL2A1 | 217404_s_at | (D) | | | (I) vertebral | (I) | | 8 | NS |
| Collagen Type II | | DE/4 | | | disc | PAG | | | |
| Alpha 1 Chain | | 54% | | | Neurological | Chronic | | | |
| | | | | | Pain ⁶ | Neuropathic | | | |
| | | | | | | Pain ¹³ | | | |
| COMT | 216204_at | (D) | Neurological Pain ²⁵, ²⁶ | | (D) Blood | | | 8 | NS |
| Catechol-O- | | DE/4 | Chronic Pain | | Chronic Pain, | | | | |
| Methyltransferase | | 54% | MSK ^{27 28, 29 30, 31, 32, 33, 34, 35, 36, 37} | | MSK ⁴² | | | | |
| | | | Pain, Acute, | | | | | | |
| | | | Thermal ³⁸ | | | | | | |
| | | | Treatments ³⁹ | | | | | | |
| | | | Pain MSK ²⁹, ²⁸, ²⁷ | | | | | | |
| | | | Pain ⁴⁰ | | | | | | |
| | | | Morphine ⁴¹ | | | | | | |
| COMT | 213981_at | (D) | Neurological | | (D) blood | | | 8 | NS |
| Catechol-O- | | DE/4 | Pain ²⁵, ²⁶ | | Chronic Pain, | | | | |
| Methyltransferase | | 54% | Chronic Pain | | MSK ⁴² | | | | |
| | | | MSK ^{27 28, 29 30, 31, 32, 33, 34, 35, 36, 37} | | | | | | |
| | | | Pain, Acute, Thermal ³⁸ | | | | | | |
| | | | Treatments ³⁹ | | | | | | |
| | | | Pain MSK ²⁹, ²⁸, ²⁷ | | | | | | |
| | | | Pain ⁴⁰ | | | | | | |
| | | | Morphine ⁴¹ | | | | | | |
| DCAF12 | 224789_at | (D) | | | (I) Whole blood | | | 8 | NS |
| DDB1 And CUL4 | | DE/6 | | | Neurological, | | | | |
| Associated Factor | | 86% | | | Pain ² | | | | |
| 12 | | | | | | | | | |
| EDN1 | 1564630_at | (I) | Fibromyalgia ⁴³ | | (I) | | | 8 | 8.69E−02 |
| Endothelin 1 | | AP/4 | | | Blister fluid | | | | Stepwise |
| | | 56% | | | Chronic Pain ⁴⁴ | | | | |
| FAM134B | 218510_x_at | (I) | Chronic, | | (I) vertebral | | | 8 | NS |
| Family With | | DE/4 | Neuropathic Pain ⁴⁵ | | disc | | | | |
| Sequence | | 51%; (I) | | | Neurological | | | | |
| Similarity 134 | | AP/2 | | | Pain ⁶ | | | | |
| Member B | | 34% | | | | | | | |
| GBP1 | 231578_at | (I) | Fibromyalgia ⁴⁶ | (D) | | | | 8 | 3.26E−01 |
| Guanylate Binding | | DE/2 | | Neurological | | | | | Stepwise |
| Protein 1 | | 37% | | Pain ¹ | | | | | |
| HLA-DRB1 | 208306_x_at | (I) | Migraine⁴⁷ | | (I) Whole blood | | | 8 | NS |
| Major | | AP/4 | | | Neurological | | | | |
| Histocompatibility | | 52% | | | Pain ² | | | | |
| Complex, Class II, | | | | | | | | | |
| DR Beta 1 | | | | | | | | | |
| HTR2A | 211616_s_at | (D) | Neurological, Pain ⁴⁸ | | (D) whole | | | 8 | NS |
| 5- | | DE/4 | Chronic, MSK ³¹, ⁴⁹, ⁵⁰ | | blood, | | | | |
| Hydroxytryptamine | | 52% | Fibromyalgia ⁵¹, ⁵², ⁵³ | | Neuropathic ⁷ | | | | |
| Receptor 2A | | | Pain, Acute, | | | | | | |
| | | | disease/lesion ⁵⁴ | | | | | | |
| | | | Pain ⁴⁰, ⁵⁵ | | | | | | |
| LOXL2 | 228808_s_at | (D) | | | (I) vertebral | (I) | | 8 | NS |
| Lysyl Oxidase Like | | DE/4 | | | disc | PFC | | | |
| 2 | | 59% | | | Neurological | Chronic | | | |
| | | | | | Pain ⁶ | Neuropathic | | | |
| | | | | | | Pain ¹³ | | | |
| LY9 | 231124_x_at | (I) | | | | (D) | | 8 | NS |
| Lymphocyte | | DE/6 | | | | NAC | | | |
| Antigen 9 | | 90% | | | | Chronic | | | |
| | | | | | | Neuropathic | | | |
| | | | | | | Pain ¹³ | | | |
| NF1 | 212676_at | (I) | Migraine ⁵⁶ | | (I) vertebral | | | 8 | NS |
| Neurofibromin 1 | | DE/4 | | | disc | | | | |
| | | 59% | | | Neurological | | | | |
| | | | | | Pain ⁶ | | | | |
| PNOC | 205901_at | (I) | | | (D) vertebral | (I) | | 8 | NS |
| Prepronociceptin | | DE/4 | | | disc | PAG | | | |
| | | 62% | | | Neurological | Chronic | | | |
| | | | | | Pain ⁶ | Neuropathic | | | |
| | | | | | (I) whole blood | Pain ¹³ | | | |
| | | | | | Neuropathic | | | | |
| | | | | | Pain ⁷ | | | | |
| SHMT1 | 217304_at | (D) | Musculoskeletal | (D) | | | | 8 | NS |
| Serine | | DE/2 | Pain ⁵⁷ | Neurological | | | | | |
| Hydroxymethyltransferase 1 | | 43% | | Pain ¹ | | | | | |
| TCF15 | 207306_at | (D) | | | | (I) | | 8 | NS |
| Transcription | | DE/6 | | | | PFC | | | |
| Factor 15 (Basic | | 94% | | | | Chronic | | | |
| Helix-Loop-Helix) | | | | | | Neuropathic | | | |
| | | | | | | Pain ¹³ | | | |
| TOP3A | 214300_s_at | (D) | | (D) | | | | 8 | NS |
| Topoisomerase | | DE/4 | | Neurological | | | | | |
| (DNA) III Alpha | | 51% | | Pain ¹ | | | | | |
| TSPO | 202096_s_at | (I) | Neuraxial Pain⁵⁸ | | (I) vertebral | (I) | | 8 | NS |
| Translocator | | DE/2 | | | disc | PAG | | | |
| Protein | | 38% | | | Neurological | Neuropathic | | | |
| | | | | | Pain ⁶ | Pain ¹³ | | | |
| | | | | | | (I) | | | |
| | | | | | | DRG) | | | |
| | | | | | | Neurological | | | |
| | | | | | | Pain ¹⁰ | | | |
| VEGFA | 212171_x_at | (I) | Neuraxial Pain⁵⁹ | | (I) | | | 8 | NS |
| Vascular | | AP/4 | | | Blood Steroid ⁶⁰ | | | | |
| Endothelial Growth | | 65% | | | (I) | | | | |
| Factor A | | | | | Chronic Pain ⁶¹ | | | | |
| | | | | | (I) | | | | |
| | | | | | serum Acute | | | | |
| | | | | | Pain MSK ⁶² | | | | |
| WNK1 | 1555068_at | (D) | Chronic Neuropathic | | | | | 8 | NS |
| WNK Lysine | | DE/6 | Pain ⁶³ | | | | | | |
| Deficient Protein | | 92% | Pain ⁴⁰ | | | | | | |
| Kinase 1 | | | | | | | | | |
| ZNF429 | 1561270_at | (D) | Pain MSK ⁶⁴ | (I) | | | | 8 | NS |
| Zinc Finger Protein | | DE/2 | Analgesia ⁶⁵ | Neurological | | | | | |
| 429 | | 37% | | Pain ¹ | | | | | |
| ZYX | 238016_s_at | (D) | | | (I) Whole blood | (I) | | 8 | NS |
| Zyxin | | DE/4 | | | Neurological | PAG | | | |
| | | 57% | | | Pain ² | Chronic | | | |
| | | | | | | Neuropathic | | | |
| | | | | | | Pain ¹³ | | | |
| (AF087971) | 1561067_at | (I) | | | | | | 6 | NS |
| PBRM1 | | AP/6 | | | | | | | |
| Polybromo 1 | | 90% | | | | | | | |
| (AF090920) | 234739_at | (I) | | | | | | 6 | NS |
| PPFIBP2 | | AP/6 | | | | | | | |
| PPFIA Binding | | 94% | | | | | | | |
| Protein 2 | | | | | | | | | |
| (H05785) | 236913_at | (D) | | | | | | 6 | NS |
| LRRC75A | | AP/6 | | | | | | | |
| Leucine Rich Repeat | | 97% | | | | | | | |
| Containing 75A | | | | | | | | | |
| (Hs.596713) | 226138_s_at | (D) | | | | | | 6 | 6.28E−02 |
| PPP1R14B | | DE/6 | | | | | | | Stepwise |
| Protein | | 90% | | | | | | | |
| Phosphatase 1 | | | | | | | | | |
| Regulatory | | | | | | | | | |
| Inhibitor Subunit | | | | | | | | | |
| 14B | | | | | | | | | |
| (Hs.609761) | 244331_at | (D) | | | | | | 6 | NS |
| SFPQ | | DE/6 | | | | | | | |
| Splicing Factor | | 98% | | | | | | | |
| Proline And | | | | | | | | | |
| Glutamine Rich | | | | | | | | | |
| (Hs.659426) | 240599_x_at | (D) | | | | | | 6 | NS |
| PHC3 | | DE/6 | | | | | | | |
| Polyhomeotic | | 92% | | | | | | | |
| Homolog 3 | | | | | | | | | |
| (Hs.666864) | 240949_x_at | (D) | | | | | | 6 | 6.03E−04 |
| MFAP3 | | DE/6 | | | | | | | Nominal |
| Microfibril | | 81% | | | | | | | |
| Associated Protein | | | | | | | | | |
| 3 | | | | | | | | | |
| (Hs.577263) | 216444_at | (D) | | | | | | 6 | NS |
| SMURF2 (SMAD | | AP/6 | | | | | | | |
| Specific E3 | | 100% | | | | | | | |
| Ubiquitin Protein | | (D) | | | | | | | |
| Ligase 2) | | DE/4 | | | | | | | |
| | | 71% | | | | | | | |
| (Hs.696420) | 243125_x_at | (D) | | | | | | 6 | NS |
| MTERF1 | | DE/6 | | | | | | | |
| Mitochondrial | | 100% | | | | | | | |
| Transcription | | | | | | | | | |
| Termination Factor 1 | | | | | | | | | |
| CLSPN | 242150_at | (I) | | | | | | 6 | NS |
| Claspin | | AP/6 | | | | | | | |
| | | 95% | | | | | | | |
| DENND1B | 1557309_at | (I) | | | | | | 6 | NS |
| DENN Domain | | DE/6 | | | | | | | |
| Containing 1B | | 90%; (I) | | | | | | | |
| | | AP/2 | | | | | | | |
| | | 40% | | | | | | | |
| DNAJC18 | 227166_at | (I) | | | | | | 6 | NS |
| DnaJ Heat Shock | | DE/6 | | | | | | | |
| Protein Family | | 94% | | | | | | | |
| (Hsp40) Member | | | | | | | | | |
| C18 | | | | | | | | | |
| ELAC2 | 201766_at | (D) | Fibromyalgia⁶⁶ | | | | | 6 | 4.11E−02 |
| ElaC Ribonuclease | | DE/4 | | | | | | | Nominal |
| Z 2 | | 52% | | | | | | | |
| GSPT1 | 215438_x_at | (D) | | | | | | 6 | NS |
| G1 To S Phase | | DE/6 | | | | | | | |
| Transition 1 | | 94% | | | | | | | |
| HRAS | 212983_at | (I) | | | | | | 6 | NS |
| HRas Proto- | | DE/6 | | | | | | | |
| Oncogene, GTPase | | 97% | | | | | | | |
| Hs.554262 | 210703_at | (I) | | | | | | 6 | NS |
| | | AP/6 | | | | | | | |
| | | 100% | | | | | | | |
| MBNL3 | 219814_at | (D) | | | | | | 6 | NS |
| Muscleblind Like | | DE/6 | | | | | | | |
| Splicing Regulator | | 92% | | | | | | | |
| 3 | | | | | | | | | |
| MCRS1 | 202556_s_at | (I) | | | | | | 6 | NS |
| Microspherule | | DE/6 | | | | | | | |
| Protein 1 | | 90% | | | | | | | |
| OSBP2 | 1569617_at | (D) | | | | | | 6 | NS |
| Oxysterol Binding | | DE/6 | | | | | | | |
| Protein 2 | | 94% | | | | | | | |
| PIK3CD | 211230_s_at | (D) | | | | | | 6 | 1.59E−02 |
| Phosphatidylinositol- | | DE/6 | | | | | | | Nominal |
| 4,5-Bisphosphate | | 83% | | | | | | | |
| 3-Kinase Catalytic | | | | | | | | | |
| Subunit Delta | | | | | | | | | |
| PTN | 211737_x_at | (D) | | | | | | 6 | NS |
| Pleiotrophin | | DE/6 | | | | | | | |
| | | 92% | | | | | | | |
| RAB33A | 206039_at | (I) | | | | | | 6 | NS |
| RAB33A, Member | | DE/6 | | | | | | | |
| RAS Oncogene | | 90% | | | | | | | |
| Family | | | | | | | | | |
| RALGAPA2 | 231826_at | (D) | | | | | | 6 | NS |
| Ral GTPase | | DE/6 | | | | | | | |
| Activating Protein | | 97% | | | | | | | |
| Catalytic Alpha | | | | | | | | | |
| Subunit 2 | | | | | | | | | |
| SEPT7P2 | 1569973_at | (I) | | | | | | 6 | NS |
| Septin 7 | | DE/6 | | | | | | | |
| Pseudogene 2 | | 100% | | | | | | | |
| | | (I) | | | | | | | |
| | | AP/2 | | | | | | | |
| | | 39% | | | | | | | |
| SVEP1 | 236927_at | (I) | Migraine ⁵⁶ | | | (D) | | 6 | 2.17E−02 |
| Sushi, Von | | DE/2 | | | | NAC | | | Nominal |
| Willebrand Factor | | 49% | | | | Neuropathic | | | |
| Type A, EGF And | | | | | | Pain ¹³ | | | |
| Pentraxin Domain | | | | | | | | | |
| Containing 1 | | | | | | | | | |
| TNFRSF11B | 204932_at | (D) | Cancer Pain ⁶⁷ | | (I) vertebral | | | 6 | 2.67E−02 |
| TNF Receptor | | DE/2 | | | disc | | | | Nominal |
| Superfamily | | 37% | | | Neurological | | | | |
| Member 11b | | | | | Pain ⁶ | | | | |
| | | | | | (I) Serum | | | | |
| | | | | | Chronic Pain ⁶⁸ | | | | |
| YBX3 | 201160_s_at | (D) | | | | | | 6 | NS |
| Y-Box Binding | | DE/6 | | | | | | | |
| Protein 3 | | 94% | | | | | | | |
| ZNF441 | 1553193_at | (I) | | | | | | 6 | NS |
| Zinc Finger Protein | | AP/6 | | | | | | | |
| 441 | | 95% | | | | | | | |
| | | (I) | | | | | | | |
| | | DE/2 | | | | | | | |
| | | 35% | | | | | | | |
| ZNF91 | 244259_s_at | (I) | | | | | | 6 | 6.37E−01/2 |
| Zinc Finger Protein | | AP/6 | | | | | | | Stepwise |
| 91 | | 95% | | | | | | | |

(n = 60 genes, 65 probesets)—evidence for involvement in pain. (I)—increased in expression in Pain, (D)—decreased in expression. DE—differential expression, AP—Absent/Present. DRG—dorsal root ganglia.

TABLE 5
Top biomarkers for pain - Evidence for involvement in other psychiatric and related disorders.

| | | | | | | Prior | | | Prior | | |
|---|---|---|---|---|---|---|---|---|---|---|---|
| | | | | | Prior | human | Prior | Prior | Non-human | Prior | |
| | | | | | human | Brain | human | Non-human | Brain | Non-human | |
| | | | Prioritization | | genetic | expression | peripheral | genetic | expression | peripheral | |
| Gene | | | Total | | evidence | evidence | evidence | evidence | evidence | evidence | External |
| Symbol/ | | Discovery | CFG | | for | for | for | for | for | for | CFG |
| Gene | | (Change) | Score | Validation | other | other | other | other | other | other | for |
| Name | Probe | Method/ | For | Anova | disorders | disorders | disorders | disorders | disorders | disorders | Other |
| Name | set | Score | Pain | p-value | 2 pts. | 4 pts | 2 pts | 1 pt. | 2 pts. | 1 pt. | Dx |
| HTR2A | 211616_s_at | (D) | 8 | NS | Alcoholism ⁶⁹ | (D) HIP BP ⁹¹ | (D) | Anxiety ¹⁰⁶ | (D) PFC SZ ¹⁰⁷ | | 13 |
| 5-Hydroxytryptamine | | DE/4 | | | BP ^{70 71 72 70, 73, 74} | (D) HIP SZ, | Lymphocyte | | (D) Frontal | | |
| Receptor | | 52% | | | Depression ^{75-77 78} | Depression⁹² | SZ ¹⁰³ | | cortex | | |
| 2A | | | | | Mood ⁷⁹ | (D) DLPFC BP ⁹² | (D) PBMC | | Depression, | | |
| | | | | | OCD ⁸⁰ | (D) Temporal | SZ ¹⁰⁴ | | SZ ¹⁰⁸ | | |
| | | | | | Addictions ^{81, 82 83 84 85} | Cortex SZ ⁹³ | (D) Platelets | | (D) PFC | | |
| | | | | | Suicide ^{79, 86 87-90} | (D) HIP BP, | Suicide ¹⁰⁵ | | Hallucinogens ¹⁰⁹ | | |
| | | | | | | SZ⁹⁴Suicide ⁹⁵ | | | (D) AMY | | |
| | | | | | | (D) PFC Aging ⁹⁶ | | | PTSD ¹¹⁰ | | |
| | | | | | | (D) frontal | | | (I) AMY | | |
| | | | | | | cortex Suicide ⁹⁷ | | | Depression¹¹¹ | | |
| | | | | | | (D) BA46 | | | | | |
| | | | | | | Suicide ⁹⁸ | | | | | |
| | | | | | | (D) Brain BP ⁹⁹ | | | | | |
| | | | | | | (I) | | | | | |
| | | | | | | AMY, Frontopolar | | | | | |
| | | | | | | cortex Suicide ¹⁰⁰ | | | | | |
| | | | | | | (D) PFC SZ¹⁰¹ | | | | | |
| | | | | | | (D) DLFPC | | | | | |
| | | | | | | Suicide ¹⁰² | | | | | |
| CDK6 | 224847_at | (I) | 8 | NS | Circadian | (I) PFC SZ ¹¹⁶ | (I) | | (I) AMY | | 10 |
| Cyclin | | DE/4 | | | abnormalities ¹¹² | (I) Brain SZ ¹¹⁷ | lymphoblastoid | | MDD ¹²¹ | | |
| Dependent | | 63% | | | Longevity ¹¹³, ¹¹⁴ | | ASD ¹¹⁸ | | | | |
| Kinase 6 | | | | | Alcohol ¹¹⁵ | | (I)Blood | | | | |
| | | | | | | | Female | | | | |
| | | | | | | | Suicide ¹¹⁹ | | | | |
| | | | | | | | (I) Blood M- | | | | |
| | | | | | | | BP Suicide ¹²⁰ | | | | |
| CDK6 | 224851_at | (I) | 8 | NS | Circadian | (I) PFC SZ ¹¹⁶ | (I) | | (I) AMY | | 10 |
| Cyclin | | DE/4 | | | abnormalities ¹¹² | (I) Brain SZ ¹¹⁷ | lymphoblastoid | | MDD ¹²¹ | | |
| Dependent | | 56% | | | Longevity ¹¹³, ¹¹⁴ | | ASD ¹¹⁸ | | | | |
| Kinase 6 | | (I) | | | Alcohol ¹¹⁵ | | (I)Blood | | | | |
| | | AP/2 | | | | | Female | | | | |
| | | 42% | | | | | Suicide ¹¹⁹ | | | | |
| | | | | | | | (I) Blood M- | | | | |
| | | | | | | | BP Suicide ¹²⁰ | | | | |
| HLA-DQB1 | 212998_x_at | (I) | 8 | NS | Longevity ¹²², ¹²³ | (I) Superior | (I) Blood SZ ¹²⁷ | | (I) CP, NAC | | 10 |
| Major | 211656_x_at | DE/4 | | NS | SZ ^{124, 125} | temporal cortex | (I) Blood | | (D) AMY | | |
| Histocompatibility | | (I) | | | | (BA 22) SZ ¹²⁶ | Suicide^{128 129} | | Alcoholism ¹³³ | | |
| Complex, | | DE/4 | | | | | (I) PBMC | | | | |
| Class II, | | 59% | | | | | Stress ¹³⁰ | | | | |
| DQ Beta 1 | | | | | | | PTSD ¹³¹ | | | | |
| | | | | | | | (I) | | | | |
| | | | | | | | Leukocytes | | | | |
| | | | | | | | Depression¹³² | | | | |
| WNK1 | 1555068_at | (D) | 8 | NS | Depression ¹³⁴ | (D) NAC | (D) Blood | | (D) PFC | | 10 |
| WNK | | DE/6 | | | | Alcohol ¹³⁵ | Suicide ¹²⁹, ¹²⁰ | | (male) BP, | | |
| Lysine | | 92% | | | | | | | Stress ¹³⁶ | | |
| Deficient | | | | | | | | | | | |
| Protein | | | | | | | | | | | |
| Kinase 1 | | | | | | | | | | | |
| (AF087971) | 1561067_at | (I) | 6 | NS | CNV, MDD ¹³⁷ | (I) DLPFC BP ¹³⁹ | (I) Blood | | (I) AMY | | 10 |
| PBRM1 | | AP/6 | | | Bp ^{138-140 141-143} | | Hallucinations¹⁴⁷ | | MDD ¹²¹ | | |
| Polybromo 1 | | 90% | | | Mood, | | (I) Blood | | (I) | | |
| | | | | | Psychosis ¹⁴⁴ | | Mood ¹⁴⁸ | | AMY(male) | | |
| | | | | | Depression ¹³⁹ | | (I) Blood | | BP, Stress ¹³⁶ | | |
| | | | | | MDD ^{139 140} | | Male Suicide ¹²⁹ | | (I) Brain | | |
| | | | | | SZ ^{141, 145} | | (I) Blood | | Stimulants ¹⁴⁹ | | |
| | | | | | Longevity¹⁴⁶ | | Female | | | | |
| | | | | | | | Suicide ¹¹⁹ | | | | |
| (Hs.666604) | 240949_x_at | (D) | 6 | 6.03E−04/4 | SZ ¹²⁴ | (D)Superior | (D)Blood | | (D) | | 10 |
| MFAP3 | | DE/6 | | Nominal | | frontal cortex | Suicide ¹²⁹, ¹²⁰ | | AMY | | |
| Microfibril | | 81% | | | | Alcohol ¹⁵⁰ | | | Stress ¹²¹ | | |
| Associated | | | | | | | | | | | |
| Protein 3 | | | | | | | | | | | |
| CCND1 | 208712_at | (D) | 8 | NS | | (D) Frontal | (D) | Addiction | (D) | | 9 |
| Cyclin D1 | | DE/4 | | | | motor cortex | Peripheral | Alcohol ¹⁵⁵ | Amygdala) | | |
| | | 57% | | | | Alcohol ¹⁵¹ | blood Stress ¹⁵⁴ | | Hallucinogens ¹⁵⁶ | | |
| | | | | | | (D) | | | (D) Amygdala | | |
| | | | | | | hippocampus | | | Addiction | | |
| | | | | | | Alcohol ¹⁵² | | | Alcohol ¹³³ | | |
| | | | | | | (D) ACC | | | | | |
| | | | | | | MDD ¹⁵³ | | | | | |
| CNTN1 | 1554784_at | (D) | 10 | NS | BP, SZ ¹⁵⁷; ¹⁵⁸ | (D) Brain BP ⁹⁹ | (D) | | | | 8 |
| Contactin 1 | | DE/4 | | | MDD ¹³⁴ | (D) HIP BP ¹⁶⁰ | lymphocyte | | | | |
| | | 52% | | | Suicide ¹⁵⁹ | (D) Forebrain | SZ ¹⁶⁴ | | | | |
| | | | | | | neural | (D) Blood | | | | |
| | | | | | | progenitor cells | Female | | | | |
| | | | | | | SZ ¹⁶¹ | Suicide ¹¹⁹ | | | | |
| | | | | | | (D) supragenual | | | | | |
| | | | | | | (BA24) anterior | | | | | |
| | | | | | | cingulated | | | | | |
| | | | | | | cortex SZ ¹⁶² | | | | | |
| | | | | | | (D) anterior | | | | | |
| | | | | | | PFC SZA ¹⁶³ | | | | | |
| GBP 1 | 231578_at | (I) | 8 | 3.26E−01/2 | | (I) | (I) | | (I) | | 8 |
| Guanylate | | DE/2 | | Stepwise | | Hippocampus, | leukocytes | | hippocampal | | |
| Binding | | 37% | | | | amygdala, | PTSD ¹⁶⁸ | | and | | |
| Protein 1 | | | | | | gyrus cinguli, | | | prefrontal | | |
| | | | | | | pons MDD ¹⁶⁵ | | | cortex MDD ¹⁶⁵ | | |
| | | | | | | (I) amygdala | | | | | |
| | | | | | | SZ ¹⁶⁶ | | | | | |
| | | | | | | (I) left side | | | | | |
| | | | | | | superior frontal | | | | | |
| | | | | | | gyrus SZ ¹⁶⁷ | | | | | |
| | | | | | | (I) Brain | | | | | |
| | | | | | | Suicide ¹⁶⁵ | | | | | |
| HLA-DQB1 | 211654_x_at | (I) | 8 | NS | | (I) superior | (I) | | (I) Caudate | | 8 |
| Major | | DE/2 | | | | temporal cortex | monocytes | | putamen | | |
| Histocompatibility | | 40% | | | | SZ ¹²⁶ | Stress ¹³⁰ | | Addiction | | |
| Complex, | | | | | | | (I) | | Alcohol¹³³ | | |
| Class II, | | | | | | | PBMC PTSD ¹³¹ | | | | |
| DQ Beta 1 | | | | | | | | | | | |
| PNOC | 205901_at | (I) | 7 | NS | Addictions ¹⁶⁹ | (I) DLPFC | (I) | | (I) NAC | | 8 |
| Prepronociceptin | | DE/4 | | | | BP, SZ ¹⁷⁰ | Fibroblasts | | Stress ¹⁷³ | | |
| | | 62% | | | | (I) AMY, | SZ ¹⁷² | | (I) Amygdala | | |
| | | | | | | cingulate cortex | | | MDD ¹¹¹ | | |
| | | | | | | MDD ¹⁷¹ | | | | | |
| | | | | | | (I) Forebrain | | | | | |
| | | | | | | neural cells | | | | | |
| | | | | | | SZ ¹⁶¹ | | | | | |
| GSPT1 | 215438_x_at | (D) | 6 | NS | | (D) Brain BP ⁹⁹ | (D) Blood | | (D) AMY | | 8 |
| G1 To S | | DE/6 | | | | | Suicide ¹²⁹ | | Depression¹²¹ | | |
| Phase | | 94% | | | | | (D) | | | | |
| Transition 1 | | | | | | | Leukocytes | | | | |
| | | | | | | | Depression¹³² | | | | |
| (Hs.609761) | 244331_at | (D) | 6 | NS | | NAC altered | (D) Blood | | (D) VT | | 8 |
| SFPQ | | DE/6 | | | | MDD ¹⁷⁴ | Female | | Hallucinogens ¹⁵⁶ | | |
| Splicing | | 98% | | | | (D) superior | Suicide ¹¹⁹ | | (D) PFC | | |
| Factor | | | | | | frontal cortex | | | (male) | | |
| Proline | | | | | | Alcohol ¹⁵⁰ | | | Stress, BP ¹³⁶ | | |
| And | | | | | | (D) PFC MDD ¹⁷⁵ | | | (D) Brain | | |
| Glutamine | | | | | | | | | Alcohol | | |
| Rich | | | | | | | | | Addiction¹⁷⁶ | | |
| ZNF91 | 244259_s_at | (I) | 6 | 6.37E−01/2 | Circadian | (I) Temporal | (I) Blood | | | | 8 |
| Zinc | | AP/6 | | Stepwise | abnormalities ¹¹² | cortex | PTSD ¹⁷⁹ | | | | |
| Finger | | 95% | | | | Alcoholism ¹⁷⁷ | | | | | |
| Protein 91 | | | | | | (I) DLPFC PTSD ¹⁷⁸ | | | | | |
| COMT | 216204_at | (D) | 8 | NS | OCD ^{180 181 182 183-185 186} | | (D) Blood | SZ^{230, 231} | (D) PFC | | 7 |
| Catechol-O- | 213981_at | DE/4 | | NS | BP ^{187 182 188 189,} | | Alcoholism ²²⁷ | Alcoholism ¹⁵⁵ | Anxiety, | | |
| Methyltransferase | | 54% | | | ^{190 191, 192} | | (D) Blood | | OCD, SZ²³² | | |
| | | (D) | | | Anxiety^{193 194 195 196 197} | | SZ ²²⁸ | | (D) Brain | | |
| | | DE/4 | | | ^{198 199 200 201} | | (D) | | Anxiety ²³³ | | |
| | | | | | SZ ^{202 203 204 205 206} | | Leukocytes | | (D) Male HIP, | | |
| | | | | | ^{207 192, 208 209} | | SZ ²²⁹ | | AMY Anxiety ²³⁴ | | |
| | | | | | Aggression ^{210 211 212} | | (D) PBMC | | | | |
| | | | | | Suicide ^{213 214 215 216 217} | | Stress ¹³⁰ | | | | |
| | | | | | Thermal ²¹⁸ | | (D) Blood | | | | |
| | | | | | Stimulants²¹⁹ | | Suicide¹¹⁹ | | | | |
| | | | | | Intellect²²⁰ | | | | | | |
| | | | | | Mood ²⁰⁹ | | | | | | |
| | | | | | ADHD ¹⁸⁶ | | | | | | |
| | | | | | Depression^{78 221-223} | | | | | | |
| | | | | | PTSD ^{224 225} | | | | | | |
| | | | | | Alcohol²²⁶ | | | | | | |
| VEGFA | 212171_x_at | (I) | 8 | NS | | (I) CA3/2 | (I) | MDD ²⁴⁰ | | | 7 |
| Vascular | | AP/4 | | | | Stratum oriens | monocytes | | | | |
| Endothelial | | 65% | | | | SZ ²³⁵ | Stress ¹³⁰ | | | | |
| Growth | | | | | | (I) Prefrontal | (I) | | | | |
| Factor A | | | | | | cortex SZ ²³⁶ | plasma MDD ²³⁸ | | | | |
| | | | | | | (I) | (I) | | | | |
| | | | | | | hippocampus | plasma BP ²³⁹ | | | | |
| | | | | | | SZ ²³⁷ | | | | | |
| (H05785) | 236913_at | (D) | 6 | NS | | (D) Brain BP ⁹⁹ | (D) Blood | Alcohol | | | 7 |
| LRRC75A | | AP/6 | | | | (D) DLPFC SZ ²⁴¹ | Male BP | Addiction¹⁵⁵ | | | |
| Leucine Rich | | 97% | | | | | Suicide ¹²⁰ | | | | |
| Repeat | | | | | | | | | | | |
| Containing | | | | | | | | | | | |
| 75A | | | | | | | | | | | |
| CALCA | 210727_at | (D) | 7 | NS | | (D) Frontal, | | | (D) Medullae | | 6 |
| Calcitonin | | DE/4 | | | | motor cortex | | | Oblongata | | |
| Related | | 54% | | | | Alcohol¹⁵¹ | | | Anxiety ²⁴² | | |
| Polypeptide | | | | | | | | | | | |
| Alpha | | | | | | | | | | | |
| LOXL2 | 228808_s_at | (D) | 7 | NS | | (D) anterior | (D) Male-BP | | | | 6 |
| Lysyl | | DE/4 | | | | PFC BP ¹⁶³ | Suicide¹²⁰ | | | | |
| Oxidase | | 59% | | | | | | | | | |
| Like 2 | | | | | | | | | | | |
| HRAS | 212983_at | (I) | 6 | NS | BP, SZ ¹⁵⁷ | | mRNA | | (I) NAC | | 6 |
| HRas | | DE/6 | | | Longevity ²⁴³ | | Suicide ²⁴⁴ | | Alcohol ²⁴⁵ | | |
| Proto- | | 97% | | | | | | | | | |
| Oncogene, | | | | | | | | | | | |
| GTPase | | | | | | | | | | | |
| (Hs.696420) | 243125_x_at | (D) | 6 | NS | | (D) DPFC BA 46 | (D) Blood | | | | 6 |
| MTERF1 | | DE/6 | | | | PTSD ²⁴⁶ | Universal | | | | |
| Mitochondrial | | | | | | | Suicide ¹²⁰ | | | | |
| Transcription | | | | | | | | | | | |
| Termination | | | | | | | | | | | |
| Factor 1 | | | | | | | | | | | |
| PTK3CD | 211230_s_at | (D) | 6 | 1.59E−02/4 | Longevity ²⁴⁷ | | (D) PBMC | | (D) NAC | | 6 |
| Phosphatidylinositol- | | DE/6 | | Nominal | SZ ²⁴⁸ | | Stress ¹³⁰ | | Alcohol ¹³³ | | |
| 4,5-Bisphosphate | | 83% | | | | | (D) Blood | | | | |
| 3-Kinase | | | | | | | Suicide ¹²⁰ | | | | |
| Catalytic | | | | | | | mRNA | | | | |
| Subunit | | | | | | | Suicide ²⁴⁴ | | | | |
| Delta | | | | | | | | | | | |
| PTN | 211737_x_at | (D) | 6 | NS | SZ ^{145 249} | | mRNA | | (D) HIP | | 6 |
| Pleiotrophin | | DE/6 | | | | | Suicide ²⁴⁴ | | Stress²⁵⁰ | | |
| | | 92% | | | | | | | | | |
| YBX3 | 201160_s_at | (D) | 6 | NS | | (D) DLPFC | (D) Blood | | | | 6 |
| Y-Box | | DE/6 | | | | BP, SZ ¹⁷⁰ | Male Suicide ¹²⁹ | | | | |
| Binding | | 94% | | | | | | | | | |
| Protein 3 | | | | | | | | | | | |
| NF1 | 212676_at | (I) | 8 | NS | | Differentially | | Addiction | (I) VS | | 5 |
| Neurofibromin 1 | | DE/4 | | | | expressed ACC | | Alcohol ¹⁵⁵ | PTSD ¹¹⁰ | | |
| | | 59% | | | | (BA 24) BP ²⁵¹ | | | | | |
| SVEP1 | 236927_at | (I) | 6 | 2.17E−02/4 | | (I) | | Alcohol ¹⁵⁵ | | | 5 |
| Sushi, Von | | DE/2 | | Nominal | | Hippocampus | | | | | |
| Willebrand | | 49% | | | | SZ ²⁵² | | | | | |
| Factor | | | | | | | | | | | |
| Type A, | | | | | | | | | | | |
| EGF And | | | | | | | | | | | |
| Pentraxin | | | | | | | | | | | |
| Domain | | | | | | | | | | | |
| Containing 1 | | | | | | | | | | | |
| (Hs.677263)Smurf2 | 216444_at | (D) | 6 | NS | | | (D) Blood | | (D) VM PFC | Intervertebral | 5 |
| SMAD | | AP/6 | | | | | Suicide ¹²⁹, ¹²⁰ | | Stress ²⁵³ | disc | |
| Specific | | 100% | | | | | | | | Aging ²⁵⁴ | |
| E3 | | (D) | | | | | | | | | |
| Ubiquitin | | DE/4 | | | | | | | | | |
| Protein | | 71% | | | | | | | | | |
| Ligase 2 | | | | | | | | | | | |
| ASTN2 | 1554816_at | (I) | 8 | 1.71E−01 | Stimulants²⁵⁵ | | (I) Female | | | | 4 |
| Astrotactin 2 | | DE/6 | | Stepwise | SZ^{256 257 206} | | Blood | | | | |
| | | 83% | | | Autism²⁵⁸ | | Suicide¹¹⁹ | | | | |
| | | | | | Autism | | | | | | |
| | | | | | CNV²⁵⁹ | | | | | | |
| | | | | | BP²⁵⁷ | | | | | | |
| CASP6 | 209790_s_at | (I) | 8 | NS | | (I) Dorsolateral | | | | | 4 |
| Caspase 6 | | DE/4 | | | | prefrontal | | | | | |
| | | 51% | | | | cortex BP ²⁶⁰ | | | | | |
| FAM134B | 218510_x_at | (I) | 8 | NS | Antisocial | | (I) Male BP | | (I) VT | | 4 |
| Family | | DE/4 | | | Personality ²⁶¹ | | SI, Universal | | Hallucinogens ¹⁵⁶ | | |
| With | | 51%; | | | | | SI¹²⁰ | | | | |
| Sequence | | (I) | | | | | | | | | |
| Similarity | | AP/2 | | | | | | | | | |
| 134 | | 34% | | | | | | | | | |
| Member B | | | | | | | | | | | |
| HLA-DQB1 | 210747_at | (D) | 8 | NS | | | (D) | | (D) Amygdala | | 4 |
| Major | | DE/2 | | | | | leukocytes | | Addictions, | | |
| Histocompatibility | | 44% | | | | | Stress, ²⁶² | | Alcohol ¹³³ | | |
| Complex, | | | | | | | | | | | |
| Class II, | | | | | | | | | | | |
| DQ Beta 1 | | | | | | | | | | | |
| ZYX | 238016_s_at | (D) | 7 | NS | | | (D) Blood | | (D) AMY | | 4 |
| Zyxin | | DE/4 | | | | | MDD ²⁶³ | | MDD ²⁶⁴ | | |
| | | 57% | | | | | | | | | |
| DNAJC18 | 227166_at | (I) | 6 | NS | | ACC (BA 24) | | | | | 4 |
| DnaJ | | DE/6 | | | | BP ²⁶⁵ | | | | | |
| Heat | | | | | | | | | | | |
| Shock | | | | | | | | | | | |
| Protein | | | | | | | | | | | |
| Family | 202556_s_at | (I) | 6 | NS | | (I) Pituitary | | | | | 4 |
| (Hsp40) | | DE/6 | | | | Depression²⁶⁶ | | | | | |
| Member | | | | | | | | | | | |
| C18 | | | | | | | | | | | |
| MCRS1 | | | | | | | | | | | |
| Microspherule | | | | | | | | | | | |
| Protein 1 | | | | | | | | | | | |
| OSBP2 | 1569617_at | (D) | 6 | NS | SZ ²⁶⁷ | | (D) Blood | | | | 4 |
| Oxysterol | | DE/6 | | | | | Suicide ^{128, 129} | | | | |
| Binding | | | | | | | (D) SH-SY5Y | | | | |
| Protein 2 | | | | | | | cells Cocaine ²⁶⁸ | | | | |
| RAB33A | 206039_at | (I) | 6 | NS | | (I) Frontal | | | | | 4 |
| RAB33A, | | DE/6 | | | | Cortex Alcohol ²⁶⁹ | | | | | |
| Member | | | | | | (I) Stress²⁷⁰ | | | | | |
| RAS | | | | | | (I)PFC, ACC, MDD¹⁵³ | | | | | |
| Oncogene | | | | | | | | | | | |
| Family | | | | | | | | | | | |
| TSPO | 202096_s_at | (I) | 6 | NS | | (I) Forebrain | | | | | 4 |
| Translocator | | DE/2 | | | | neural | | | | | |
| Protein | | 38% | | | | progenitor cells | | | | | |
| | | | | | | SZ ¹⁶¹ | | | | | |
| GNG7 | 1566643_a_at | (D) | 10 | 6.81E−02/2 | | | | | (D) | | 2 |
| G Protein | | DE/4 | | Stepwise | | | | | NAC | | |
| Subunit | | 59% | | | | | | | Alcohol ²⁷¹ | | |
| Gamma 7 | | | | | | | | | (D) | | |
| | | | | | | | | | PFC | | |
| | | | | | | | | | Hallucinogens ¹⁵⁶ | | |
| | | | | | | | | | (D) | | |
| | | | | | | | | | PFC (male) | | |
| | | | | | | | | | BP/Stress ¹³⁶ | | |
| | | | | | | | | | (D) | | |
| | | | | | | | | | AMY | | |
| | | | | | | | | | MDD ¹¹¹ | | |
| COL27A1 | 225293_at | (D) | 8 | 7.47E−01/2 | Tourette | | | | | | 2 |
| Collagen | | DE/4 | | Stepwise | syndrome ²⁷² | | | | | | |
| Type | | 79% | | | | | | | | | |
| XXVII | | | | | | | | | | | |
| Alpha 1 | | | | | | | | | | | |
| Chain | | | | | | | | | | | |
| DCAF12 | 224789_at | (D) | 8 | NS | | | (D) SH-SY5Y | | | | 2 |
| DDB1 And | | DE/6 | | | | | cells Cocaine ²⁶⁸ | | | | |
| CUL4 | | 86% | | | | | (D) Blood | | | | |
| Associated | | | | | | | Universal | | | | |
| Factor 12 | | | | | | | Suicide¹²⁰ | | | | |
| SHMT1 | 217304_at | (D) | 8 | NS | | | (D) Blood | | | | 2 |
| Serine | | DE/2 | | | | | Suicide ¹²⁹, ¹²⁰ | | | | |
| Hydroxymethyl- | | 43% | | | | | | | | | |
| transferase 1 | | | | | | | | | | | |
| (Hs.596713) | 226138_s_at | (D) | 6 | 6.28E−02 | | | | | (D) parietal | | 2 |
| PPP1R14B | | DE/6 | | Stepwise | | | | | cortex SZ²⁷³ | | |
| Protein | | 90% | | | | | | | | | |
| Phosphatese 1 | | | | | | | | | | | |
| Regulatory | | | | | | | | | | | |
| Inhibitor | | | | | | | | | | | |
| Subunit | | | | | | | | | | | |
| 14B | | | | | | | | | | | |
| CCDC65C | 219018_s_at | (D) | 6 | NS | | | (D) Male | | | | 2 |
| Coiled- | | DE/6 | | | | | Blood | | | | |
| Coil | | 94% | | | | | Suicide ¹²⁹ | | | | |
| Domain | | | | | | | | | | | |
| Containing | | | | | | | | | | | |
| 85C | | | | | | | | | | | |
| CLSPN | 242150_at | (I) | 6 | NS | | | (I) Blood | | | | 2 |
| Claspin | | AP/6 | | | | | Suicide ^{119, 129} | | | | |
| | | 95% | | | | | | | | | |
| ELAC2 | 201766_at | (D) | 6 | 4.11E−02/4 | Autism²⁷⁴ | | | | | | 2 |
| ElaC | | DE/4 | | Nominal | | | | | | | |
| Ribonuclease | | 52% | | | | | | | | | |
| Z 2 | | | | | | | | | | | |
| Hs.554262 | 210703_at | (I) | 6 | NS | | | (I) Blood | | | | 2 |
| | | AP/6 | | | | | Universal | | | | |
| | | | | | | | Suicide¹²⁰ | | | | |
| (Hs.65942)PHC3 | 240599_x_at | (D) | 6 | NS | | | (D) Blood | | | | 2 |
| Polyhomeotic | | DE/6 | | | | | Female | | | | |
| Homolog 3 | | | | | | | Suicide ¹¹⁹ | | | | |
| LY9 | 231124_x_at | (I) | 6 | NS | | | (D) Blood | | | | 2 |
| Lymphocyte | | DE/6 | | | | | Stress²⁷⁵ | | | | |
| Antigen 9 | | 90% | | | | | | | | | |
| MBNL3 | 219814_at | (D) | 6 | NS | | | (D) Blood | | | | 2 |
| Muscleblind | | DE/6 | | | | | Hallucinations ¹⁴⁷ | | | | |
| Like | | 92% | | | | | | | | | |
| Splicing | | | | | | | | | | | |
| Regulator 3 | | | | | | | | | | | |
| RALGAPA2 | 231826_at | (D) | 6 | NS | BP ⁷⁰ | | | | | | 2 |
| Ral | | DE/6 | | | | | | | | | |
| GTPase | | 97% | | | | | | | | | |
| Activating | | | | | | | | | | | |
| Protein | | | | | | | | | | | |
| Catalytic | | | | | | | | | | | |
| Alpha | | | | | | | | | | | |
| Subunit 2 | | | | | | | | | | | |
| SEPT7P2 | 1569973_at | (I) | 6 | NS | | | (I) Blood | | | | 2 |
| Septin 7 | | DE/6 | | | | | Suicide¹¹⁹ | | | | |
| Pseudogene 2 | | 100% | | | | | | | | | |
| | | (I) | | | | | | | | | |
| | | AP/2 | | | | | | | | | |
| | | 39% | | | | | | | | | |
| TCF15 | 207306_at | (D) | 6 | NS | | | (D) Blood | | | | 2 |
| Transcription | | DE/6 | | | | | Suicide ¹²⁹, ¹²⁰ | | | | |
| Factor | | 94% | | | | | | | | | |
| 15 (Basic | | | | | | | | | | | |
| Helix- | | | | | | | | | | | |
| Loop- | | | | | | | | | | | |
| Helix) | | | | | | | | | | | |
| TNFRSF11B | 204932_at | (D) | 4 | 2.67E−02/4 | | | | | (D) | | 2 |
| TNF | | DE/2 | | Nominal | | | | | Hippocampus | | |
| Receptor | | 37% | | | | | | | Stress¹²¹ | | |
| Superfamily | | | | | | | | | (D) PFC | | |
| Member | | | | | | | | | Stress ²⁵³ | | |
| 11b | | | | | | | | | (D) HC PTSD ¹¹⁰ | | |
| HLA-DRB1 | 208306_x_at | (I) | | NS | | | (I) | | | | 2 |
| Major | | AP/4 | | | | | leukocytes | | | | |
| Histocompatibility | | 52% | | | | | Stress ²⁶² | | | | |
| Complex, | | | | | | | (I) | | | | |
| Class II, | | | | | | | Blood PTSD ²⁷⁶ | | | | |
| DR Beta 1 | | | | | | | | | | | |
| CCDC144B | 1557366_at | (D) | 10 | NS | | | | | | | 0 |
| Coiled- | | DE/4 | | | | | | | | | |
| Coil | | 56% | | | | | | | | | |
| Domain | | | | | | | | | | | |
| Containing | | | | | | | | | | | |
| 144B | | | | | | | | | | | |
| (Pseudogene) | | | | | | | | | | | |
| COL2A1 | 217404_s_at | (D) | 7 | NS | | | | | | | 0 |
| Collagen | | DE/4 | | | | | | | | | |
| Type II | | 54% | | | | | | | | | |
| Alpha 1 | | | | | | | | | | | |
| Chain | | | | | | | | | | | |
| (AF090920) | 234739_at | (I) | 6 | NS | | | | | | | 0 |
| PPFIBP2 | | AP/6 | | | | | | | | | |
| PPFIA | | 94% | | | | | | | | | |
| Binding | | | | | | | | | | | |
| Protein 2 | | | | | | | | | | | |
| DBMND1B | 1557309_at | (I) | 6 | NS | | | | | | | 0 |
| DENN | | DE/6 | | | | | | | | | |
| Domain | | 90% | | | | | | | | | |
| Containing 1B | | | | | | | | | | | |
| ZNF441 | 1553193_at | (I) | 6 | NS | | | | | | | 0 |
| Zinc | | AP/6 | | | | | | | | | |
| Finger | | 95% | | | | | | | | | |
| Protein | | (I) | | | | | | | | | |
| 441 | | DE/2 | | | | | | | | | |
| | | 35% | | | | | | | | | |
| TOP3A | 214300_s_at | (D) | 4 | NS | | | | | | | 0 |
| Topoisomerase | | DE/4 | | | | | | | | | |
| (DNA) III | | 51% | | | | | | | | | |
| Alpha | | | | | | | | | | | |
| ZNF429 | 1561270_at | (D) | 2 | NS | | | | | | | 0 |
| Zinc | | DE/2 | | | | | | | | | |
| Finger | | 37% | | | | | | | | | |
| Protein | | | | | | | | | | | |
| 429 | | | | | | | | | | | |

In the same direction of expression.
(I)—increased in expression in Pain,
(D)—decreased in expression.
DE—differential expression,
AP—Absent/Present.

TABLE 6
Biological Pathway Analysis:

| | Ingenuity Pathways |
|---|---|
| DAVID GO Functional Annotation | (Fold change) |

| | | |
|---|---|---|
| Biological Processes | KEGG Pathways | Top |

| | | | | | P- | | | | P- | Canonical | P- | |
|---|---|---|---|---|---|---|---|---|---|---|---|---|
| A. | # | Term | Count | % | Value | Term | Count | % | Value | Pathways | Value | Overlap |
| 60 Pain Genes | 1 | regulation of | 11 | 18.6 | 1.10E−06 | Focal adhesion | 7 | 11.9 | 7.20E−05 | Hereditary | 3.36E−05 | 3.5% |
| (n = 60 | | homeostatic | | | | | | | | Breast Cancer | | 5/144 |
| Genes, 65 | | process | | | | | | | | Signaling | | |
| probesets) | 2 | epithelial cell | 8 | 13.6 | 9.60E−05 | PI3K-Akt | 8 | 13.6 | 1.60E−04 | Ovarian | 3.36E−05 | 3.5% |
| | | proliferation | | | | signaling | | | | Cancer | | 5/144 |
| | | | | | | pathway | | | | Signaling | | |
| | 3 | T cell receptor | 6 | 10.2 | 1.70E−04 | Non-small cell | 4 | 6.8 | 1.00E−03 | Non-Small Cell | 4.53E−05 | 5.2% |
| | | signaling pathway | | | | lung cancer | | | | Lung Cancer | | 4/77 |
| | | | | | | | | | | Signaling | | |
| | 4 | aging | 7 | 11.9 | 2.30E−04 | Pancreatic | 4 | 6.8 | 1.60E−03 | Glioblastoma | 5.89E−05 | 3.1% |
| | | | | | | cancer | | | | Multiform | | 5/162 |
| | | | | | | | | | | Signaling | | |
| | 5 | negative | 12 | 20.3 | 2.50E−04 | Glioma | 4 | 6.8 | 1.60E−03 | HER-2 | 7.65E−05 | 4.5% |
| | | regulation of | | | | | | | | Signaling in | | 4/88 |
| | | multicellular | | | | | | | | Breast | | |
| | | organismal | | | | | | | | Cancer | | |
| | | process | | | | | | | | | | |

| | David | | | | | Ingenuity Pathways Disease | | |
|---|---|---|---|---|---|---|---|---|
| | | | | | P- | Diseases and | P- | # |
| B. | # | Term | Count | % | Value | Disorders | Value | Molecules |
| 60 Pain | 1 | Mood disorders | 5 | 8.5 | 2.00E−05 | Neurological | 2.5E−05- | 30 |
| Genes | | | | | | Disease | 3.26E−08 | |
| (n = 60 | 2 | Head and Neck Cancer | 6 | 10.2 | 2.10E−05 | Cancer | 2.50E−03- | 54 |
| Genes, 65 | | | | | | | 9.87E−08 | |
| probesets) | | | | | | | | |
| | 3 | Arthritis, | 7 | 11.9 | 4.40E−05 | Organismal | 2.56E−03- | 55 |
| | | Rheumatoid/ | | | | Injury and | 9.87E−08 | |
| | | Rheumatoid | | | | Abnormalities | | |
| | | Arthritis | | | | | | |
| | 4 | Autism | 9 | 15.3 | 4.40E−05 | Reproductive | 1.86E−03- | 37 |
| | | | | | | System | 1.79E−07 | |
| | | | | | | Disease | | |
| | 5 | Glomerulonephritis, | 6 | 10.2 | 6.30E−05 | Renal and | 1.44E−03- | 16 |
| | | IGA | | | | Urological | 1.11E−06 | |
| | | | | | | Disease | | |

TABLE 7
Pharmacogenomics. Top list biomarkers in datasets that are targets of existing drugs and are modulated by them in opposite direction.

| | | Discovery | Prioritization | | | | | | | |
|---|---|---|---|---|---|---|---|---|---|---|
| Gene Symbol/ | | (Change) | Total CFG | Validation | | | | | | |
| Gene Name | | Method/ | Score For | Anova | Pain | | | Mood | | |
| Name | Probeset | Score | Pain | p-value | Medications | Omega-3 | Antidepressants | Stabilizers | Antipsychotics | Others |
| CNTN1 | 1554784_at | (D) | 10 | NS | | | | | (I) VT | |
| Contactin 1 | | DE/4 | | | | | | | Clozapine ¹⁵⁶ | |
| | | 52% | | | | | | | | |
| GNG7 | 1566643_a_at | (D) | 10 | 6.81E−02/2 | | (I)Brain | | | | |
| G Protein | | DE/4 | | Stepwise | | Omega-3 | | | | |
| Subunit | | 59% | | | | fatty | | | | |
| Gamma 7 | | | | | | acids²⁷⁷ | | | | |
| | | | | | | (I)AMY(females) | | | | |
| | | | | | | Omega-3 | | | | |
| | | | | | | fatty²⁷⁸ | | | | |
| ASTN2 | 1554816_at | (I) | 8 | 1.71E−01 | | | | | Antipsychotics ²⁷⁹ | |
| Astrotactin 2 | | DE/6 | | Stepwise | | | | | | |
| | | 83% | | | | | | | | |
| CDK6 | 224851_at | (I) | 8 | NS | | | | | | palbociclib, |
| Cyclin | | DE/4 | | | | | | | | ribociclib, |
| Dependent | | 56% | | | | | | | | abemaciclib, |
| Kinase 6 | | (I) | | | | | | | | letrozole/ |
| | | AP/2 | | | | | | | | palbociclib, |
| | | 42% | | | | | | | | FLX925, |
| | | | | | | | | | | fulvestrant/ |
| | | | | | | | | | | palbociclib, |
| | | | | | | | | | | trilaciclib, |
| | | | | | | | | | | G1T38, |
| | | | | | | | | | | letrozole/ |
| | | | | | | | | | | ribociclib, |
| | | | | | | | | | | abemaciclib/ |
| | | | | | | | | | | fulvestrant, |
| | | | | | | | | | | alvocidib |
| CDK6 | 224847_at | (I) | 8 | NS | | | | | | |
| Cyclin | | DE/4 | | | | | | | | |
| Dependent | | 63% | | | | | | | | |
| Kinase 6 | | | | | | | | | | |
| COL27A1 | 225293_at | (D) | 8 | 7.47E−01/2 | | | | (I) | | |
| Collagen | | DE/4 | | Stepwise | | | | AMY | | |
| Type XXVII | | 79% | | | | | | Lithium ²⁸⁰ | | |
| Alpha 1 Chain | | | | | | | | | | |
| COMT | 213981_at; | (D) | 8 | NS | Morphine ⁴¹ | | | Mood | (I) VT | |
| Catechol-O- | 216204_at | DE/4 | | | Thermal ²¹⁸ | | | Stabilizers²⁸¹ | Clozapine¹⁵⁶ | |
| Methyltransferase | | 54% | | | | | | | | |
| DDB1 And | 224789_at | (D) | 8 | NS | | (I) | | | (I) | |
| CUL4 | | DE/6 | | | | Lymphocytes | | | Lymphocytes | |
| Associated | | 86% | | | | (females) | | | Clozapine¹⁵⁶ | |
| Factor 12 | | | | | | Omega-3 | | | | |
| | | | | | | fatty | | | | |
| | | | | | | acids²⁷⁸ | | | | |
| FAM1348 | 218510_x_at | (I) | 8 | NS | | (D) | | | | |
| Family With | | DE/4 | | | | Lymphocytes | | | | |
| Sequence | | 51%; | | | | (females) | | | | |
| Similarity 134 | | (I) | | | | Omega-3 | | | | |
| Member B | | AP/2 | | | | fatty | | | | |
| | | 34% | | | | acids²⁷⁸ | | | | |
| GBP 1 | 231578_at | (I) | 8 | 3.26E−01/2 | | (D) Blood | | | | |
| Guanylate | | DE/2 | | Stepwise | | Omega-3 | | | | |
| Binding | | 37% | | | | fatty | | | | |
| Protein 1 | | | | | | acids ²⁷⁸ | | | | |
| Major | 210747_at | (D) | 8 | NS | | | | | (I)Blood | |
| Histocompatibility | | DE/2 | | | | | | | Benzodiazepines ²⁸² | |
| Complex, | | 44% | | | | | | | | |
| Class II, DQ | | | | | | | | | | |
| Beta 1 | | | | | | | | | | |
| HLA-DQB1 | 211654_x_at | (I) | 8 | NS | | | | | (D)PFC | |
| Major | | DE/2 | | | | | | | Antipsychotics ²⁸³ | |
| Histocompatibility | | 40% | | | | | | | | |
| Complex, | | | | | | | | | | |
| Class II, DQ | | | | | | | | | | |
| Beta 1 | | | | | | | | | | |
| HLA-DQB1 | 211656_x_at; | (I) | 8 | NS | | | | | (D) PFC | |
| Major | 212998_x_at | DE/4 | | | | | | | Antipsychotics ²⁸³ | |
| Histocompatibility | | 59% | | | | | | | | |
| Complex, | | | | | | | | | | |
| Class II, DQ | | | | | | | | | | |
| Beta 1 | | | | | | | | | | |
| HLA-DRB1 | 208306_x_at | (I) | 8 | NS | | | | | (D)PFC | apolizumab |
| Major | | AP/4 | | | | | | | Antipsychotics ²⁸³ | |
| Histocompatibility | | 52% | | | | | | | | |
| Complex, | | | | | | | | | | |
| Class II, DR | | | | | | | | | | |
| Beta 1 | | | | | | | | | | |
| HTR2A | 211616_s_at | (D) | 8 | NS | | | | | | Hallucinogens |
| 5-Hydroxytryptamine | | DE/4 | | | | | | | | |
| Receptor 2A | | 52% | | | | | | | | |
| NF1 | 212676_at | (I) | 8 | NS | | | (D) cerebral | | | |
| Neurofibromin 1 | | DE/4 | | | | | cortex | | | |
| | | 59% | | | | | Fluoxetine SSRI ²⁸⁴ | | | |
| SHMT1 | 217304_at | (D) | 8 | NS | | | | | (I)VT | |
| Serine | | DE/2 | | | | | | | Clozapine ¹⁵⁶ | |
| Hydroxymethyl- | | 43% | | | | | | | | |
| transferase 1 | | | | | | | | | | |
| Topoisomerase | 214300_s_at | (D) | 8 | NS | | (I)Brain | | | | |
| (DNA) III | | DE/4 | | | | Omega-3 | | | | |
| Alpha | | 51% | | | | fatty | | | | |
| | | | | | | acids²⁷⁷ | | | | |
| VEGFA | 212171_x_at | (I) | 8 | NS | | | | (D) | (D) HIP | Anti-cancer |
| Vascular | | AP/4 | | | | | | lymphoblastoid | and | mABs |
| Endothelial | | 65% | | | | | | cell | cerebellum | |
| Growth | | | | | | | | cultures | Olanzapine ²⁸⁶ | |
| Factor A | | | | | | | | Lithium, | | |
| | | | | | | | | Valproate ²⁸⁵ | | |
| WNK1 | 1555068_at | (D) | 8 | NS | | (I) | (I) cingulate | | | |
| WNK Lysine | | DE/6 | | | | Lymphocytes | cortex SSRI | | | |
| Deficient | | 92% | | | | (females) | (Fluoxetine)²⁶⁴ | | | |
| Protein | | | | | | Omega-3 | | | | |
| Kinase 1 | | | | | | fatty | | | | |
| | | | | | | acids²⁷⁸ | | | | |
| CALCA | 210727_at | (D) | 7 | NS | | (I) HIP | | (I) | | |
| Calcitonin | | DE/4 | | | | (males) | | Schneider | | |
| Related | | 54% | | | | Omega-3 | | 2 cells | | |
| Polypeptide | | | | | | fatty | | Lithium²⁸⁷ | | |
| Alpha | | | | | | acids²⁷⁸ | | | | |
| ZYX | 238016_s_at | (D) | 7 | NS | | | | | (I) | |
| Zyxin | | DE/4 | | | | | | | Lymphocytes | |
| | | 57% | | | | | | | Clozapine ¹⁵⁶ | |
| (HD5785) | 236913_at | (D) | 6 | NS | | | | | (I) HIP | |
| LRRC75A | | AP/6 | | | | | | | Clozapine¹⁵⁶ | |
| Leucine Rich | | 97% | | | | | | | | |
| Repeat Containing | | | | | | | | | | |
| 75A | | | | | | | | | | |
| (Hs.596713) | 226138_s_at | (D) | 6 | 6.28E−02 | | | | (I) | | |
| PPP1R14B | | DE/6 | | Stepwise | | | | Schneider | | |
| Protein | | 90% | | | | | | 2 (S2) | | |
| Phosphatase | | | | | | | | cells, | | |
| Regulatory | | | | | | | | Lithium ²⁸⁷ | | |
| Inhibitor | | | | | | | | | | |
| Subunit 14B | | | | | | | | | | |
| (Hs.609761) | 244331_at | (D) | 6 | NS | | (I) HIP | (I) basal | | (I) PFC | |
| SFPQ | | DE/6 | | | | (males) | forebrain TCA²⁸⁸ | | Clozapine¹⁵⁶ | |
| Splicing | | 98% | | | | Mood, | | | | |
| Factor Proline | | | | | | Omega-3 | | | | |
| And | | | | | | fatty | | | | |
| Glutamine | | | | | | acids²⁷⁸ | | | | |
| Rich | | | | | | | | | | |
| DENND1B | 1557309_at | (I) | 6 | NS | | (D) Brain | | | | |
| DENN | | DE/6 | | | | Omega-3 | | | | |
| Domain | | 90%; | | | | fatty | | | | |
| Containing 1B | | (I) | | | | acids²⁷⁷ | | | | |
| | | AP/2 | | | | | | | | |
| | | 40% | | | | | | | | |
| GSPT1 | 215438_x_at | (D) | 6 | NS | | | | (I) CP | | |
| G1 To S | | DE/6 | | | | | | Valproate ²⁸⁹ | | |
| Phase | | 94% | | | | | | | | |
| Transition 1 | | | | | | | | | | |
| HRAS | 212983_at | (I) | 6 | NS | | | | | | ISIS 2503 |
| HRas Proto- | | DE/6 | | | | | | | | |
| Oncogene, | | 97% | | | | | | | | |
| GTPase | | | | | | | | | | |
| LY9 | 231124_x_at | (I) | 6 | NS | | (D) Brain | | | | |
| Lymphocyte | | DE/6 | | | | Omega-3 | | | | |
| Antigen 9 | | 90% | | | | fatty | | | | |
| | | | | | | acids ²⁷⁷ | | | | |
| PTK3CD | 211230_s_at | (D) | 6 | 1.59E−02/4 | | | | (I) | (I) VT | |
| Phosphatidylinositol- | | DE/6 | | Nominal | | | | Lymphoblastoid | Clozapine¹⁵⁶ | |
| 4,5-Bisphosphate | | 83% | | | | | | cells | | |
| 3-Kinase | | | | | | | | Lithium, | | |
| Catalytic | | | | | | | | Valproate ²⁸⁵ | | |
| Subunit Delta | | | | | | | | | | |
| PTN | 211737_x_at | (D) | 6 | NS | | (I) HIP | | | | |
| Pleiotrophin | | DE/6 | | | | (males) | | | | |
| | | 92% | | | | Omega-3 | | | | |
| | | | | | | fatty | | | | |
| | | | | | | acids ²⁷⁸ | | | | |
| | | | | | | (I) | | | | |
| | | | | | | fronto- | | | | |
| | | | | | | temporo- | | | | |
| | | | | | | parietal | | | | |
| | | | | | | cortex | | | | |
| | | | | | | Antipsychotics(risperidone) ²⁹⁰ | | | | |
| SVEP1 | 236927_at | (I) | 6 | 2.17E−02/4 | | (D)Brain | | | | |
| Sushi, Von | | DE/2 | | Nominal | | Omega-3 | | | | |
| Willebrand | | 49% | | | | fatty | | | | |
| Factor Type | | | | | | acids²⁷⁷ | | | | |
| A, EGF And | | | | | | | | | | |
| Pentraxin | | | | | | | | | | |
| Domain | | | | | | | | | | |
| Containing 1 | | | | | | | | | | |
| TSPO | 202096_s_at | (I) | 6 | NS | | | | | | CGS-8216, |
| Translocator | | DE/2 | | | | | | | | dexamethasone/ |
| Protein | | 38% | | | | | | | | olanzapine, |
| | | | | | | | | | | fluoxetine/ |
| | | | | | | | | | | olanzapine, |
| | | | | | | | | | | estazolam, |
| | | | | | | | | | | clorazepate, |
| | | | | | | | | | | eszopiclone, |
| | | | | | | | | | | temazepam, |
| | | | | | | | | | | zolpidem, |
| | | | | | | | | | | chlordiazepoxide, |
| | | | | | | | | | | lorazepam, |
| | | | | | | | | | | olanzapine, |
| | | | | | | | | | | triazolam, |
| | | | | | | | | | | flumazenil, |
| | | | | | | | | | | clonazepam, |
| | | | | | | | | | | flurazepam, |
| | | | | | | | | | | midazolam, |
| | | | | | | | | | | flunitrazepam, |
| | | | | | | | | | | alprazolam, |
| | | | | | | | | | | zaleplon, |
| | | | | | | | | | | SSR180575, |
| | | | | | | | | | | PK 11195 |
| YBX3 | 201160_s_at | (D) | 6 | NS | | | (I) c. elegans | | | |
| Y-Box Binding | | DE/6 | | | | | mianserin ²⁹¹ | | | |
| Protein 3 | | 94% | | | | | | | | |

1-22. (canceled)

23. A method for diagnosing current pain and risk of future pain, treating pain, and monitoring response to treatment in an individual in need thereof, comprising:
(a) obtaining a biological sample from the individual and quantifying the amounts of a panel of one or more biomarkers in the biological sample,
(b) quantifying the amounts of the biomarker(s) in a clinically relevant population to generate a reference expression level;
(c) comparing the amounts of the biomarker(s) in the biological sample with the amounts present in the reference standard to generate a score for each biomarker; whereas the biomarkers in the panel comprise one or more of:
GNG7, CNTN1, CCDC144B, MFAP3, COMT, ZYX, MTERF1, COL27A1, CALCA, PPP1R14B, ELAC2, TCF15, TOP3A, LRRC75A, COL2A1, PIK3CD, TNFRSF11B, DCAF12, WNK1, SFPQ, PHC3, CCDC85C, GSPT1, LOXL2, MBNL3, PTN, RALGAPA2, YBX3, CCND1, HTR2A, SHMT1, OSBP2, ZNF429, SMURF2, and combinations thereof, wherein the expression level of the biomarker(s) in the sample is increased relative to a reference expression level, denoting increased pain; or
LY9, GBP1, CASP6, RAB33A, HRAS, ASTN2, HLA-DQB1, PNOC, CLSPN, Hs.554262, SVEP1, ZNF91, CDK6, EDN1, PPFIBP2, DNAJC18, HLA-DRB1, SEPT7P2, VEGFA, PBRM1, ZNF441, NF1, TSPO, DENND1B, MCRS1, FAM134B, and combinations thereof, wherein the expression level of the biomarker(s) in the sample is decreased relative to a reference expression level, denoting increased pain;
(d) generating a score for the panel, based on the scores of the biomarker(s) in the panel; with the values for the increased in expression (risk) biomarkers being added, and the resulting values for the decreased in expression (protective) biomarkers being subtracted;
(e) determining a reference score for the panel in a clinically normal relevant population;
(f) identifying a difference between the score of the panel of biomarker(s) in the sample and the reference score of the panel of biomarker(s);
(g) diagnosing the individual as having current pain, and/or future pain risk based on the difference between the biomarker panel score of the individual relative to the biomarker panel score of reference;
(h) treating pain by administering to the individual identified as having current pain, and/or future pain risk a therapeutically effective amount of a specific therapeutic drug (s), based on the specific biomarkers whose scores indicate that they are changed in the individual compared to a reference standard;
(i) monitoring response to treatment by obtaining a biological sample from the individual after starting treatment, determining a score for the panel of biomarker(s), and comparing it to a reference score for the panel of biomarkers; and
(j) determining that the treatment is effective if the difference between the score of the panel of biomarker(s) in the sample and the reference score of the panel of biomarker(s) has decreased compared to the difference that existed before treatment.

24. The method of claim 23, wherein the biomarkers are quantified on samples taken on two or more occasions from the individual, (a) wherein one of the two or more occasions is prior to commencement of therapy and one of the two or more occasions is after commencement of therapy; (b) wherein an effect the therapy has on an individual is determined based a change in the amount of the biomarkers in samples taken on two or more occasions, (c) wherein the occasion after commencement of therapy is following therapy, (d) wherein samples are taken at intervals over the remaining life, or a part thereof of the individual.

25. The method of claim 23, wherein before the step of generating the biomarker panel score, each biomarker is given a weighted coefficient, wherein the weighted coefficient is related to the importance of said each biomarker in assessing and predicting pain risk.

26. The method of claim 23, wherein the biological sample is a peripheral tissue sample or a fluid, such as cerebrospinal fluid, whole blood, blood serum, plasma, urine, saliva, or other bodily fluid, or breath, condensed breath, or an extract or purification therefrom, or dilution thereof.

27. The method of claim 23, wherein the biomarker expression level measures RNA or protein of the biomarker in the biological sample.

28. The method of claim 23, wherein the therapeutic is one or more known pain medications or one or more psychiatric medications, selected from: ketamine and other dissociants; lithium, valproate, and other mood stabilizers; clozapine, olanzapine, chlorpromazine, haloperidol, paliperidone, iloperidone, asenapine, cariprazine, lurasidone, quetiapine, risperidone, aripiprazole, brexpiprazole, and other antipsychotics; amoxapine, paroxetine, mirtazapine, buspirone, fluoxetine, mianserin, amitriptyline, trimipramine, and other antidepressants; benzodiazepines and other anxiolytics; docosahexaenoic acid and other omega-3 fatty acids; and combinations thereof.

29. The method of claim 23, wherein the therapeutic is one or more from a group of new method of use/repurposed drugs, consisting of: SC-560, pyridoxine, methylergometrine, LY-294002, haloperidol, cytisine, cyanocobalamin, apigenin, beta-escin, amoxapine, ISIS 2503, (-)-Gallocatechin gallate, EICOSATRIENOIC ACID (20:3 n-3), LFM-A13, Picrotoxinin, INDAPAMIDE, BRD-K15318909, BRD-K53011428 BRD-K35100517, MLS-0454435.0001, NCGC00181213-02, ST003833, STOCK2S-84516, MLS-0390932.0001, BRD-K98143437, BRD-A00993607, BRD-K68103045, BRD-K90700939, triamterene, PSEUDOEPHEDRINE HYDROCHLORIDE, DOCOSAHEXAENOIC ACID (22:6 n-3), Evoxine, Gavestinel, Mometasone furoate, ZM 241385, and combinations thereof.

30. The method of claim 23, whereas the result is determining intensity of pain in a subject.

31. The method of claim 23, wherein the result is predicting a future medical care facility visit for pain-related complaints.

32. The method of claim 23, wherein the assessing of mood, anxiety, psychosis and combinations thereof in the individual stratifies the individual in one of the following subtypes: a predominantly psychotic subtype, possibly related to mis-connectivity and increased perception of pain centrally, and a predominantly anxious subtype, possibly related to reactivity and increased physical health reasons for pain peripherally.

33. A method for identifying a blood biomarker for pain, the method comprising:
obtaining a first biological sample from a subject and administering a first pain intensity test to the subject;
obtaining a second biological sample from the subject and administering a second pain intensity test to the subject;
identifying a first cohort of subjects by identifying subjects having a change from low pain intensity to high pain intensity as determined by a difference between the first pain intensity test and the second pain intensity test; and
identifying candidate biomarkers in the first cohort by identifying biomarkers having a change in expression between the first biological sample and the second biological sample.

34. The method of claim 33 further comprising prioritizing the candidate biomarkers by identifying candidate biomarkers known to be associated with pain.

35. The method of claim 33, wherein the pain intensity test is selected from the group consisting of Visual Analog Scale for Pain (VAS Pain), Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), Measure of Intermittent and Constant Osteoarthritis Pain (ICOAP), and combinations thereof.
